SEQUENCE LISTING (123 sequences)

SEQ ID NO: 1 - DNA, 864 bp, Brachysprira hyodysenteriae
atgaaaggaatcgtattagcaggcggaagcggaacaagactttatccaatgacaaatgttatatcaaaacagcttttgccaatatacgataaaccaatggtatattatccattatcagttttaatgctttcaaaaattaaagatatacttgtaatatctacagaaaatgatacaccgctttttaaaaaattattaaaagacggaagtcaatggggatgcaatattgaatatgctgtgcaaaaagcacctaatggacttgcagaagcatttataataggagaaagttttataggaaaagataatgtttctatgatattaggtgataatatattttttggacaaagcttttcttctgtattagaaaaaagtgctaagatagataatggggctgttatatttgcttatcaagtaaaagatcctgaacgtttcggtatagttgaaatagataaagattataatgctttatctatagaagaaaaacctaaaaatccaaagtctaattatgctgttacaggtctttatttttatgataatgatgtagttaatatttcaaaaaatattaagccttctgcaagaggggagcttgaaataacagatgttaataaagtatatttagaaaataaaaaacttaaagttgaagtactaggacgcggatttgcttggcttgataccggtacaagagatagtcttattcaggcttctcaatttgtgtctgctgtagaaaatattgaaggtatgcagatagcttgtttagaagaaatagcttataataataaatggattgatgcttcaaaaatagaagagactgcatcaaaattgaaaaactcagaatatggtaaatacttaattaatttaattaaatag

SEQ ID NO: 2 - DNA, 1071 bp, Brachysprira hyodysenteriae
atgagaaattttaataatattttagttactggtggatgcggttttataggatcaaactttataagatacatattagaaaaatctgattataaaggcaatattataaatatagatgccttaacttatgcaggaaacagagaaaatttaatagatatagaaaaaaaatataaagatagatatttctttgaaaaagttaatatttgtgatgataaaaaaataaatgaaatattttcaaaatataatcctgattgtatagtgcattttgctgcagaaagtcatgttgaccgttcaatatacggaccaaaagattttcttgaaactaatataatgggaacttttattttattagaagctgctcgtaaattatgggaaaataataaagaaaataaactatttcatcatataagtacagacgaagtttacggatctttaggtgaaacaggttatttttatgaaactactccttatgatcctagaagtccttactcatcatcaaaagcttcaagtgatcatattgtaaaagcatattatcatacttataatctgcctgttacaatatctaattgcagcaacaattatggcccttatcaattccctgaaaaattaatacctttaatgatttctaatatagtagaagaaaaagatcttcctgtttatggagatggaaaaaatataagagattggatatttgttgaagatcataataatgcagtaattgatataatacataaaggcagggtaggagagacctacaatatcggcggagaaaacgagattactaatattgatatggtaaatattttatgtgagaaattatctgctaaaatgaataaagaaaaagattattataaaaaattaatcaaatttgtaaaagacagagccggtcatgacagaagatatgctataaactgcgataaaataaaaaaagaattaggatgggaaagaaaatacgatttcaataccgccattgatataactatagattggtatttaaataacaaacaatggatagataatattaaaagcggagaatataaaaaatggattgagttaaactactcgtctaaataa

SEQ ID NO: 3 - DNA, 579 bp, Brachysprira hyodysenteriae
atgccttttacatttaaaaaacttgatataaacggactttttatagcagaacctcgtatctttggagattcaagaggttatttcctagaaacatattctgagaaagatttttttgaagctggaatcaaagaaaaatttgtacaagataatcaatctaaatcaacaaaaaatgtattaagaggacttcattatcagaaaaaatattgtcaggctaaattggttagagtattagaaggtgaagtatttgatgttgcagtagatttgagaaatgattctccaacttttggaaaatattatggggtattattaacagaagaaaataaaaagcagttctttattcctaaaggttttgcacatggatttattgttctaagcgaatcagctgtttttgcttataaatgttctgacttttatcaccctgaagaagagggtggtataatatataatgataaaaatattaatatagattggacttgcgatactaaagatgtattagtatctgataaagatttaaaattaccagagtttgacaaaaataaaaaatatttttcattagatggaaattggattggagaataa

SEQ ID NO: 4 - DNA, 861 bp, Brachysprira hyodysenteriae
atgatttgggtaattggtaaaaatggaatgcttgctcaagatatactcaaagcattcaataaaaataatatagaatatacttcaacagcttctaatatagatattactaatattgatatacttaataactttataaaagataaaaatattaaaacaataataaattgttctgcatatacaaaagtagatttagcagaagatgaaaaagatatttgctataaagttaatggagaaggtgtaaaaaatataacagaaatagcttctaatataaatgcagatttaattcatttttctactgattatgtatttgatggagaaaataacaaaccatacaatgaagaagataaaactaatccaataaatatttacggcaaaagtaaattagagggtgaaaaatatgctttattttataataaagcaatagttattagagtatcttggctatatggaatttatggaaaaaattttgtatatactatgattaatttgatgaatagtaaagaaagcataaaagtagttaatgatcaatttggatctcctacatatacagaagatgttgctatggtaatatatgatattataaaaaataacaattttgattacggactttatcattatactaatgaaggtaatatatcatggtatgattttgctaaaactatatataataaaggaaaagagtataatattataaataatgattgcaaagtaaatccatgtactacagaagaatatccaaccaaagctaaaagacctaaatacagtgttttatctgtagaaaaaataaaaagatatgttaaaatatataattatgaagatagcttaaatagtttttttaacatatatcaaaaaaataaataa

SEQ ID NO: 5 - DNA, 1872 bp, Brachysprira hyodysenteriae
atgaatgataaatatgttatatgttcaattacaacaggatttacagatcaactcataaattttgctacagcaatatatattagagaaaaatataaaaaaaatgtaaaattagatatatcatggttttctgtttatgaaaatacagattatttgggaatagaaaaacgtaacctggaagtactaaatatatttaatgactttaattttgaaatagctacaaaaaaagaaatagatatagctttaaagtctatttttatagatggtagatatgaaaatatagataaaatattaaaatccaataaacatatatatgttggaccatatgttggacatactttattttactatggacatattaatatttcttcttttttcaatcttgacaaatattttattaatttattaaatgaaaaacataaaaacatattaaatgacatgtcatcttttgaaagtgttactatacatataagacgaggagattatatgtatgcgttaaaaatagatggaagaaagaataatgcttctttagactatatatatacatcaataaaatatttaaataatactttagaaaataaaaatataaaattttatatattttctaataataaaaaatatataaaaaataatttaataccaatttttcaaaataataatattaattttgaaattatagaagataatcctgaatatatagatttttatttaatgactaaatgtaagcatataatatcttcattaggaaaatttgcaaaagtagcttttaaatttataaaaaatgaaaataaaatatttatagatacagacaatatttataatttgaaaaaagaatatttctataaaaatatttgtgaaaaatactgcaacataatttatgaagaatataataatataaaaacatatatatcaaaaaataatatgatatatatactagacttaattgaaaaaaataatataaagaatatatgtcaaataggcttgctagacggtttagaaactcatactctattaaaatattcagcagacaataataactgttttaatttattatgttttgaaaataatgaaagagatatagttggaatagaattaaaaaatttaacagaaaatgaaaaaaaactatttacattatatatagaacagtctcctttagatatagataattcttttaatataaaaaatatagattttatcttttttacaaaagaaaattctaatcctctagtattcatttatttaatatacttatttccatttatgaaagaagaaatcataatagtatttaatgatattataagtggtaaatattattctggaagttattgtatatttaatatgtttaatggagaaaaaaatcaattttataattttaatacaaataaatatgacaatatcggatatataaaaataaagaaacaagaattattaaatataataaaaaaaatatctgttatagaatatgatggttatataaataaattttttatgaataaattaatagataacagaaatgattattataattattttgatttagatatgtcatacattaggttaaataatttaaagcaatatatgaaggataaatttgatatagaatattcaaattatataatctctaacatagaaaataatattattaattatgaaaaacaatattataaaaaccaaaaaaataatttttattctcaaaatcaaatacatatttctaaaatagaaaatataatagataaacataataaattaattaatagtatatcttggtggatacctataaaaaaattaagagaaaatttcaaaaataaatttataagaccagaccagaccagaccagaccagaccagaccagaccagaccagacctaatatgtaa

SEQ ID NO: 6 - DNA, 981 bp, Brachysprira hyodysenteriae
atgaaaagattagccgtatttgcaggatatgataaagacaatataattgatgattatgttgtatattatattaaagaattaaaaaaaatatctgatataatctatgtatcagattgcaatatgtcagataatgaattaaaaaaaattaatgattactgcatacatatcataaatggaaggcatgaagaatatgatttcggatcatataaaagaggatatctatatgccaaagaaaataatatgcttcaaaattatgattatttaatactttgtaatgattcttgctatggacctttatttgatttggaaaacataataaataaaatgaatgattataattttggaggcatatctattagtaaagatttaaaaatagcaaatcataattatataactagcttttttatatacataaacaaaaacatatttaataaagatttctttaataattttatttatgatataaaaaaagaagaagataaaatggatatcataaaaaaatatgaatttggactttcaaaactaatgcttgataataatatagaattaaaaggcttatttaatgataatggagaatttaacagaccatactttaatccattagctctaatagaagaaggatatccttttttaaaaaaacatgtactagaaaaaaaagtaactgttcctttaaatatagatgaactaacaaatattataaaaataataaaaaataattacgatataaaattaatagttaatcatttaaatagagttgctgataaagaacaaattaaatatttatttcaaaaatataaaccatataaaaaaacatttattcatgaaaaaattttctccttatttaccagatattcaccatcaggtaaatatcaaacagtatataatttttttaattttctaactatatcaatagacaaaccaattaaagattcatacatagaaactgactataaagattttaattttttattagaaatataa

SEQ ID NO: 7 - DNA, 1218 bp, Brachysprira hyodysenteriae
atgataagttatttaaataaaaggtataaaatgaataaatgtgaatgcagtcaatgcaaaaatagagaatatgatatatcagaatatttggttcttgaagatgacttgaaaattttagataatgaaagaccactaggaatatcaggacatttaagggttaaagatgaagctatgagtttagctgaatgcattgattcatgcatagactttttagatgaacttataataacttataacacatcttcagataacacagaagaaatattaaaaaactacgaaaaaaaatatcctgaaaaaattaaactatttcattataaacctaatattataagatacaataaagatgagtataatacaaaatattcacaaatacactatttagataattattacaattatggactaattaaaataaaatacaaatattatgttaaaatagatgctgatcaaatttatttcacagataaattattaaaattaagagaattattaatattagatataaatcaaataaaattaagcaataatataaataaattattattaataaataaaatagcttggtgcatacctatcaaaaaattacgaaataaatttagaacatattgtattagaaagtatttcaataaagataaaaattatatttataatggtgatgaaaatatattatcattagatgattttattttattatctaggaaaagacattcagataaaaatatcagttatgatcttgcaggattcaatctaatattacataataataaattatatttagatacatattggcctatgaatggcggacgttttgatcatgtgctgtatcaagcatcttattctaataaatattttatgaatgaaatatttgaagtaaatcaaactcctcataatagaataattttagggatgttttgggtacattttggaaatattaaacataattttcaatatgatgattctttttatttactagaatataatgatataaaaaattctaaagtagaaaatttttctcataaaatgaaaaatattgataatgatgataaaatagaaaaatatataagagataatacttttataaaatattttgatgaggataaaaaataccttaataatgttttttataataaatacttgacaaaaccacttgactatgctataaaaaacaatgatgaattagtaaatttatatggaaaaactattattactgtaaaatga

SEQ ID NO: 8 - DNA, 795 bp, Brachysprira hyodysenteriae
atgtctagcaatataatagatacaatagcttggtggattcctattaaatccattagaataaaatttagaaatgcaacaaagaaaaagttcaataattttatttttaacaaaatagattttaactacgttaatacaaataatatagaacttatactcaataaatataatgaaatatttgtcccattaatattaagtaacgaaaaaaaatacaaaaaaaatagaaatagcgagttaataaataaatatttaactggtttaaaaggtgttgaaataggaggaagcgctggagctgattttggtatagacgctatacaagttaatattgtcgatgccaatcaggaacacaataattattatcaacaaacaaatattttaggttcgggatataatcaaagaactgatatcatatcatgtggtgatgtattaccatttcaagataattcaatagactttgtattctcaagtcatgtactagaacatttctatgaccctataaaagctttaaaagaatggtacagagttataaaaaaagacggatatatagctatgatagtacctcataaagaaagaacattcgataaagatagagatagaacaactttaaatgaattaatagaaagacataataaggttaaaaattataaatacaatgtggatgaaagaggaatgcatttttctgtttggataacagaagatatactagaattatgcaaatatttaaatatgaatgtaattgaatatctagatgtagatgatgctgtaggtaatggatttgctattatcataaaaaaataa

SEQ ID NO: 9 - protein, 623 aa, Brachysprira hyodysenteriae
MNDKYVICSITTGFTDQLINFATAIYIREKYKKNVKLDISWFSVYENTDYLGIEKRNLEVLNIFNDFNFEIATKKEIDIALKSIFIDGRYENIDKILKSNKHIYVGPYVGHTLFYYGHINISSFFNLDKYFINLLNEKHKNILNDMSSFESVTIHIRRGDYMYALKIDGRKNNASLDYIYTSIKYLNNTLENKNIKFYIFSNNKKYIKNNLIPIFQNNNINFEIIEDNPEYIDFYLMTKCKHIISSLGKFAKVAFKFIKNENKIFIDTDNIYNLKKEYFYKNICEKYCNIIYEEYNNIKTYISKNNMIYILDLIEKNNIKNICQIGLLDGLETHTLLKYSADNNNCFNLLCFENNERDIVGIELKNLTENEKKLFTLYIEQSPLDIDNSFNIKNIDFIFFTKENSNPLVFIYLIYLFPFMKEEIIIVFNDIISGKYYSGSYCIFNMFNGEKNQFYNFNTNKYDNIGYIKIKKQELLNIIKKISVIEYDGYINKFFMNKLIDNRNDYYNYFDLDMSYIRLNNLKQYMKDKFDIEYSNYIISNIENNIINYEKQYYKNQKNNFYSQNQIHISKIENIIDKHNKLINSISWWIPIKKLRENFKNKFIRPDQTRPDQTRPDQTRPNM

SEQ ID NO: 10 - protein, 405 aa, Brachysprira hyodysenteriae
MISYLNKRYKMNKCECSQCKNREYDISEYLVLEDDLKILDNERPLGISGHLRVKDEAMSLAECIDSCIDFLDELIITYNTSSDNTEEILKNYEKKYPEKIKLFHYKPNIIRYNKDEYNTKYSQIHYLDNYYNYGLIKIKYKYYVKIDADQIYFTDKLLKLRELLILDINQIKLSNNINKLLLINKIAWCIPIKKLRNKFRTYCIRKYFNKDKNYIYNGDENILSLDDFILLSRKRHSDKNISYDLAGFNLILHNNKLYLDTYWPMNGGRFDHVLYQASYSNKYFMNEIFEVNQTPHNRIILGMFWVHFGNIKHNFQYDDSFYLLEYNDIKNSKVENFSHKMKNIDNDDKIEKYIRDNTFIKYFDEDKKYLNNVFYNKYLTKPLDYAIKNNDELVNLYGKTIITVK

SEQ ID NO: 11 - protein, 287 aa, Brachysprira hyodysenteriae
MKGIVLAGGSGTRLYPMTNVISKQLLPIYDKPMVYYPLSVLMLSKIKDILVISTENDTPLFKKLLKDGSQWGCNIEYAVQKAPNGLAEAFIIGESFIGKDNVSMILGDNIFFGQSFSSVLEKSAKIDNGAVIFAYQVKDPERFGIVEIDKDYNALSIEEKPKNPKSNYAVTGLYFYDNDVVNISKNIKPSARGELEITDVNKVYLENKKLKVEVLGRGFAWLDTGTRDSLIQASQFVSAVENIEGMQIACLEEIAYNNKWIDASKIEETASKLKNSEYGKYLINLIK

SEQ ID NO: 12 - protein, 356 aa, Brachysprira hyodysenteriae
MRNFNNILVTGGCGFIGSNFIRYILEKSDYKGNIINIDALTYAGNRENLIDIEKKYKDRYFFEKVNICDDKKINEIFSKYNPDCIVHFAAESHVDRSIYGPKDFLETNIMGTFILLEAARKLWENNKENKLFHHISTDEVYGSLGETGYFYETTPYDPRSPYSSSKASSDHIVKAYYHTYNLPVTISNCSNNYGPYQFPEKLIPLMISNIVEEKDLPVYGDGKNIRDWIFVEDHNNAVIDIIHKGRVGETYNIGGENEITNIDMVNILCEKLSAKMNKEKDYYKKLIKFVKDRAGHDRRYAINCDKIKKELGWERKYDFNTAIDITIDWYLNNKQWIDNIKSGEYKKWIELNYSSK

SEQ ID NO: 13 - protein, 192 aa, Brachysprira hyodysenteriae
MPFTFKKLDINGLFIAEPRIFGDSRGYFLETYSEKDFFEAGIKEKFVQDNQSKSTKNVLRGLHYQKKYCQAKLVRVLEGEVFDVAVDLRNDSPTFGKYYGVLLTEENKKQFFIPKGFAHGFIVLSESAVFAYKCSDFYHPEEEGGIIYNDKNINIDWTCDTKDVLVSDKDLKLPEFDKNKKYFSLDGNWIGE

SEQ ID NO: 14 - protein, 286 aa, Brachysprira hyodysenteriae
MIWVIGKNGMLAQDILKAFNKNNIEYTSTASNIDITNIDILNNFIKDKNIKTIINCSAYTKVDLAEDEKDICYKVNGEGVKNITEIASNINADLIHFSTDYVFDGENNKPYNEEDKTNPINIYGKSKLEGEKYALFYNKAIVIRVSWLYGIYGKNFVYTMINLMNSKESIKVVNDQFGSPTYTEDVAMVIYDIIKNNNFDYGLYHYTNEGNISWYDFAKTIYNKGKEYNIINNDCKVNPCTTEEYPTKAKRPKYSVLSVEKIKRYVKIYNYEDSLNSFFNIYQKNK

SEQ ID NO: 15 - protein, 326 aa, Brachysprira hyodysenteriae
MKRLAVFAGYDKDNIIDDYVVYYIKELKKISDIIYVSDCNMSDNELKKINDYCIHIINGRHEEYDFGSYKRGYLYAKENNMLQNYDYLILCNDSCYGPLFDLENIINKMNDYNFGGISISKDLKIANHNYITSFFIYINKNIFNKDFFNNFIYDIKKEEDKMDIIKKYEFGLSKLMLDNNIELKGLFNDNGEFNRPYFNPLALIEEGYPFLKKHVLEKKVTVPLNIDELTNIIKIIKNNYDIKLIVNHLNRVADKEQIKYLFQKYKPYKKTFIHEKIFSLFTRYSPSGKYQTVYNFFNFLTISIDKPIKDSYIETDYKDFNFLLEI

SEQ ID NO: 16 - protein, 264 aa, Brachysprira hyodysenteriae
MSSNIIDTIAWWIPIKSIRIKFRNATKKKFNNFIFNKIDFNYVNTNNIELILNKYNEIFVPLILSNEKKYKKNRNSELINKYLTGLKGVEIGGSAGADFGIDAIQVNIVDANQEHNNYYQQTNILGSGYNQRTDIISCGDVLPFQDNSIDFVFSSHVLEHFYDPIKALKEWYRVIKKDGYIAMIVPHKERTFDKDRDRTTLNELIERHNKVKNYKYNVDERGMHFSVWITEDILELCKYLNMNVIEYLDVDDAVGNGFAIIIKK

SEQ ID NO: 17 - DNA, 1818 bp, Brachysprira hyodysenteriae
atgaatgataaatatgttatatgttcaattacaacaggatttacagatcaactcataaattttgctacagcaatatatattagagaaaaatataaaaaaaatgtaaaattagatatatcatggttttctgtttatgaaaatacagattatttgggaatagaaaaacgtaacctggaagtactaaatatatttaatgactttaattttgaaatagctacaaaaaaagaaatagatatagctttaaagtctatttttatagatggtagatatgaaaatatagataaaatattaaaatccaataaacatatatatgttggacatactttattttactatggacatattaatatttcttcttttttcaatcttgacaaatattttattaatttattaaatgaaaaacataaaaacatattaaattacatgtcatcttttgaaagtgttgctatacatataagacgaggagattatatgtatgcgttaaaaatagatggaagaaagaataatgcttctttagactatatatatacatcaataaaatatttaaataatactttagaaaataaaaatataaaattttatatattttctaataataaaaaatatataaaaaataatttaataccaatttttcaaaataataatattaattttgaaattatagaagataatcctgaatatatagatttttatttaatgactaaatgtaagcatataatatcttcattaggaaaatttgcaaaagtagcttttaaatttataaaaaatgaaaataaaatatttatagatacagacaatatttataatttgaaaaaagaatatttctataaaaatatttgtgaaaaatactgcaacataatttatgaagaatataataatataaaaacatatatatcaaaaaataatatgatatatatactagacttaattgaaaaaaataatataaagaatatatgtcaaataggcttgctagacggtttagaaactcatactctattaaaatattcagcagacaataataactgttttaatttattatgttttgaaaataatgaaagagatatagttggaatagaattaaaaaatttaacagaaaatgaaaaaaaactatttacattatatatagaacagtctcctttagatatagataattcttttaatataaaaaatatagattttatcttttttacaaaagaaaattctaatcctctagtattcatttatttaatatacttatttccatttatgaaagaagaaatcataatagtatttaatgatattataagtggtaaatattattctggaagttattgtatatttaatatgtttaatggagaaaaaaatcaattttataattttaatacaaataaatatgacaatatcggatatataaaaataaagaaacaagaattattaaatataataaaaaaaatatctgttatagaatatgatggttatataaataaattttttatgaataaattaatagataacagaaatgattattataattattttgatttagatatgtcatacattaggttaaataatttaaagcaatatatgaaggataaatttgatatagaatattcaaattatataatctctaacatagaaaataatattattaattatgaaaaacaatattataaaaaccaaaaaaataatttttattctcaaaatcaaatacatatttctaaaatagaaaatataatagataaacataataaattaattaatagtatatcttggtggatacctataaaagaattaagagaaaatttcaaaaataaatttataagaccagaccagacctaa

SEQ ID NO: 18 - DNA, 1902 bp, Brachysprira hyodysenteriae
atgaatgataaatatgttatatgttcaattacaacaggatttacagatcaactcataaattttgctacagcaatatatattagagaaaaatataaaaaaaatgtaaaattagatatatcatggttttctgtttatgaaaatacagattatttgggaatagaaaaacgtaacctggaagtactaaatatatttaatgactttaattttgaaatagctacaaaaaaagaaatagatatagctttaaagtctatttttatagatggtagatatgaaaatatagataaaatattaaaatccaataaacatatatatgttggaccatatgttggacatactttattttactatggacatattaatatttcttcttttttcaatcttgacaaatattttattaatttattaaatgaaaaacataaaaacatattaaatgacatgtcatcttttgaaagtgttgctatacatataagacgaggagattatatgtatgcgttaaaaatagatggaagaaagaataatgcttctttagactatatatatacatcaataaaatatttaaataatactttagaaaataaaaatataaaattttatatattttctaataataaaaaatatataaaaaataatttaataccaatttttcaaaataataatattaattttgaaattatagaagataatcctgaatatatagatttttatttaatgactaaatgtaagcatataatatcttcattaggaaaatttgcaaaagtagcttttaaatttataaaaaatgaaaataaaatatttatagatacagacaatatttataatttgaaaaaagaatatttctataaaaatatttgtgaaaaatactgcaacataatttatgaagaatataataatataaaaacatatatatcaaaaaataatatgatatatatactagacttaattgaaaaaaataatataaagaatatatgtcaaataggcttgctagacggtttagaaactcatactctattaaaatattcagcagacaataataactgttttaatttattatgttttgaaaataatgaaagagatatagttggaatagaattaaaaaatttaacagaaaatgaaaaaaaactatttacattatatatagaacagtctcctttagatatagataattcttttaatataaaaaatatagattttatcttttttacaaaagaaaattctaatcctctagtattcatttatttaatatacttatttccatttatgaaagaagaaatcataatagtatttaatgatattataagtgataaatattattctggaagttattgtatatttaatatgtttaatggagaaaaaaatcaattttataattttaatacaaatgaatatgacaatatcggatatataaaaataaagaaacaagaattattaaatataataaaaaaaatatctgttatagaatatgatggttatataaataaattttttatgaataaattaatagataacagaaatgattattataattattttgatttagatatgtcatacattaggttaaataatttaaagcaatatatgaaggataaatttgatatagaatattcaaattatataatctctaacatagaaaataatattattaattatgaaaaacaatattataaaaaccaaaaaaataatttttattccaaaaatcaaatacatatttctaaaatagaaaatataatagataaacataataaattaattaatagtatatcttggtggatacctataaaaaaattaagagaaaatttcaaaaataaatttataagaccagaccagaccagaccagaccagaccagaccagaccagaccagaccagaccagaccagaccagaccagaccagacctaatatgtaa

SEQ ID NO: 19 - DNA, 1860 bp, Brachysprira hyodysenteriae
atgaatgataaatatgttatatgttcaattacaacaggatttacagatcaactcataaattttgctacagcaatatatattagagaaaaatataaaaaaaatgtaaaattagatatatcatggttttctgtttatgaaaatacagattatttgggaatagaaaaacgtaatctggaagtactaaatatatttaatgactttaattttgaaatagctacaaaaaaagaaatagatatagctttaaagtctatttttatagatggtagatatgaaaatatagataaaatattaaaatccaataaacatatatatgttggaccatatgttggacatactttattttactatggacatattaatatttcttcttttttcaatcttgacaaatattttattaatttattaaatgaaaaacataaaaacatattaaatgacatgtcatcttttgaaagtgttgctatacatataagacgaggagattatatgtatgcgttaaaaatagatggaagaaagaataatgcttctttagactatatatatacatcaataaaatatttaaataatactttagaaaataaaaatataaaattttatatattttctaataataaaaaatatataaaaaataatttaataccaatttttcaaaataataatattaattttgaaattatagaagataatcctgaatatatagatttttatttaatgactaaatgtaagcatataatatcttcattaggaaaatttgcaaaagtagcttttaaatttataaaaaatgaaaataaaatatttatagatacagacaatatttataatttgaaaaaagaatatttctataaaaatatttgtgaaaaatactgcaacataatttatgaagaatataataatataaaaacatatatatcaaaaaataatatgatatatatactagacttaattgaaaaaaataatataaagaatatatgtcaaataggcttgctagacggtttagaaactcatactctattaaaatattcagcagacaataataactgttttaatttattatgttttgaaaataatgaaagagatatagttggaatagaattaaaaaatttaacagaaaatgaaaaaaaactatttacattatatatagaacagtctcctttagatatagataattcttttaatataaaaaatatagattttatcttttttacaaaagaaaattctaatcctctagtattcatttatttaatatacttatttccatttatgaaagaagaaatcataatagtatttaatgatattataagtgataaatattattctggaagttattgtatatttaatatgtttaatggagaaaaaaatcaattttataattttaatacaaataaatatgacaatatcggatatataaaaataaagaaacaagaattattaaatataataaaaaaaatatctgttatagaatatgatggttatataaataaattttttatgaataaattaatagataacagaaatgattattataattattttgatttagatatgtcatacattaggttaaataatttaaagcaatatatgaaggataaatttgatatagaatattcaaattatataatctttaacatagaaaataatattattaattatgaaaaacaatattataaaaaccaaaaaaataatttttattccaaaaatcaaatacatatttctaaaatagaaaatataatagataaacataataaattaattaatagtatatcttggtggatacctataaaaaaattaagagaaaatttcaaaaataaatttataagaccagaccagaccagaccagaccagaccagaccagaccagacctaa

SEQ ID NO: 20 - DNA, 1935 bp, Brachysprira hyodysenteriae
atgaatgataaatatgttatatgttcaattacaacaggatttacagatcaactcataaattttgctacagcaatatatattagagaaaaatataaaaaaaatgtaaaattagatatatcatggttttctgtttatgaaaatacagattatttgggaatagaaaaacgtaacctggaagtactaaatatatttaatgactttaattttgaaatagctacaaaaaaagaaatagatatagctttaaagtctatttttatagatggtagatatgaaaatatagataaaatattaaaatccaataaacatatatatgttggaccatatgttggacatactttattttactatggacatattaatatttcttcttttttcaatcttgacaaatattttattaatttattaaatgaaaaacataaaaacatattaaatgacatgtcatcttttgaaagtgttgctatacatataagacgaggagattatatgtatgcgttaaaaatagatggaagaaagaataatgcttctttagactatatatatacatcaataaaatatttaaataatactttagaaaataaaaatataaaattttatatattttctaataataaaaaatatataaaaaataatttaataccaatttttcaaaataataatattaattttgaaattatagaagataatcctgaatatatagatttttatttaatgactaaatgtaagcatataatatcttcattaggaaaatttgcaaaagtagcttttaaatttataaaaaatgaaaataaaatatttatagatacagacaatatttataatttgaaaaaagaatatttctataaaaatatttgtgaaaaatactgcaacataatttatgaagaatataataatataaaaacatatatatcaaaaaataatatgatatatatactagacttaattgaaaaaaataatataaagaatatatgtcaaataggcttgctagacggtttagaaactcatactctattaaaatattcagcagacaataataactgttttaatttattatgttttgaaaataatgaaagagatatagttggaatagaattaaaaaatttaacagaaaatgaaaaaaaactatttacattatatatagaacagtctcctttagatatagataattcttttaatataaaaaatatagattttatcttttttacaaaagaaaattctaatcctctagtattcatttatttaatatacttatttccatttatgaaagaagaaatcataatagtatttaatgatattataagtggtaaatattattctggaagttattgtatatttaatatgtttaatggagaaaaaaatcaattttataattttaatacaaataaatatgacaatatcggatatataaaaataaagaaacaagaattattaaatataataaaaaaaatatctgttatagaatatgatggttatataaataaattttttatgaataaattaatagataacagaaatgattattataattattttgatttagatatgtcatacattaggttaaataatttaaagcaatatatgaaggataaatttgatatagaatattcaaattatataatctctaacatagaaaataatattattaattatgaaaaacaatattataaaaaccaaaaaaataatttttattctcaaaatcaaatacatatttctaaaatagaaaatataatagataaacataataaattaattaatagtatatcttggtggatacctataaaaaaattaagagaaaatttcaaaaataaatttataagaccagaccagaccagaccagaccagaccagaccagaccagaccagaccagacctaatatgtaaagaatacatatataatatactatattacaaaataaataaattacaatatataaattaa

SEQ ID NO: 21 - DNA, 1860 bp, Brachysprira hyodysenteriae
atgaatgataaatatgttatatgttcaattacaacaggatttacagatcaactcataaattttgctacagcaatatatattagagaaaaatataaaaaaaatgtaaaattagatatatcatggttttctgtttatgaaaatacagattatttgggaatagaaaaacgtaacctggaagtactaaatatatttaatgactttaattttgaaatagctacaaaaaaagaaatagatatagctttaaagtctatttttatagatggtagatatgaaaatatagataaaatattaaaatccaataaacatatatatgttggaccatatgttggacatactttattttactatggacatattaatatttcttcttttttcaatcttgacaaatattttattaatttattaaatgaaaaacataaaaacatattaaatgacatgtcatcttttgaaagtgttactatacatataagacgaggagattatatgtatgcgttaaaaatagatggaagaaagaataatgcttctttagactatatatatacatcaataaaatatttaaataatactttagaaaataaaaatataaaattttatatattttctaataataaaaaatatataaaaaataatttaataccaatttttcaaaataataatattaattttgaaattatagaagataatcctgaatatatagatttttatttaatgactaaatgtaagcatataatatcttcattaggaaaatttgcaaaagtagcttttaaatttataaaaaatgaaaataaaatatttatagatacagacaatatttataatttgaaaaaagaatatttctataaaaatatttgtgaaaaatactgcaacataatttatgaagaatataataatataaaaacatatatatcaaaaaataatatgatatatatactagacttaattgaaaaaaataatataaagaatatatgtcaaataggcttgctagacggtttagaaactcatactctattaaaatattcagcagacaataataactgttttaatttattatgttttgaaaataatgaaagagatatagttggaatagaattaaaaaatttaacagaaaatgaaaaaaaactatttacattatatatagaacagtctcctttagatatagataattcttttaatataaaaaatatagattttatcttttttacaaaagaaaattctaatcctctagtattcatttatttaatatacttatttccatttatgaaagaagaaatcataatagtatttaatgatattataagtggtaaatattattctggaagttattgtatatttaatatgtttaatggagaaaaaaatcaattttataattttaatacaaataaatatgacaatatcggatatataaaaataaagaaacaagaattattaaatataataaaaaaaatatctgttatagaatatgatggttatataaataaattttttatgaataaattaatagataacagaaatgattattataattattttgatttagatatgtcatacattaggttaaataatttaaagcaatatatgaaggataaatttgatatagaatattcaaattatataatctctaacatagaaaataatattattaattatgaaaaacaatattataaaaaccaaaaaaataatttttattctcaaaatcaaatacatatttctaaaatagaaaatataatagataaacataataaattaattaatagtatatcttggtggatacctataaaaaaattaagagaaaatttcaaaaataaatttataagaccagaccagaccagaccagaccagaccagaccagaccagacctaa

SEQ ID NO: 22 - DNA, 1860 bp, Brachysprira hyodysenteriae
atgaatgataaatatgttatatgttcaattacaacaggatttacagatcaactcataaattttgctacagcaatatatattagagaaaaatataaaaaaaatgtaaaattagatatatcatggttttctgtttatgaaaatacagattatttgggaatagaaaaacgtaacctggaagtactaaatatatttaatgactttaattttgaaatagctacaaaaaaagaaatagatatagctttaaagtctatttttatagatggtagatatgaaaatatagataaaatattaaaatccaataaacatatatatgttggaccatatgttggacatactttattttactatggacatattaatatttcttcttttttcaatcttgacaaatattttattaatttattaaatgaaaaacataaaaacatattaaattacatgtcatcttttgaaagtgttgctatacatataagacgaggagattatatgtatgcgttaaaaatagatggaagaaagaataatgcttctttagactatatatatacatcaataaaatatttaaataatactttagaaaataaaaatataaaattttatatattttctaataataaaaaatatataaaaaataatttaataccaatttttcaaaataataatattaattttgaaattatagaagataatcctgaatatatagatttttatttaatgactaaatgtaagcatataatatcttcattaggaaaatttgcaaaagtagcttttaaatttataaaaaatgaaaataaaatatttatagatacagacaatatttataatttgaaaaaagaatatttctataaaaatatttgtgaaaaatactgcaacataatttatgaagaatataataatataaaaacatatatatcaaaaaataatatgatatatatactagacttaattgaaaaaaataatataaagaatatatgtcaaataggcttgctagacggtttagaaactcatactctattaaaatattcagcagacaataataactgttttaatttattatgttttgaaaataatgaaagagatatagttggaatagaattaaaaaatttaacagaaaatgaaaaaaaactatttacattatatatagaacagtctcctttagatatagataattcttttaatataaaaaatatagattttatcttttttacaaaagaaaattctaatcctctagtattcatttatttaatatacttatttccatttatgaaagaagaaatcataatagtatttaatgatattataagtggtaaatattattctggaagttattgtatatttaatatgtttaatggagaaaaaaatcaattttataattttaatacaaataaatatgacaatatcggatatataaaaataaagaaacaagaattattaaatataataaaaaaaatatctgttatagaatatgatggttatataaataaattttttatgaataaattaatagataacagaaatgattattataattattttgatttagatatgtcatacattaggttaaataatttaaagcaatatatgaaggataaatttgatatagaatattcaaattatataatctctaacatagaaaataatattattaattatgaaaaacaatattataaaaaccaaaaaaataatttttattctcaaaatcaaatacatatttctaaaatagaaaatataatagataaacataataaattaattaatagtatatcttggtggatacctataaaagaattaagagaaaatttcaaaaataaatttataagaccagaccagaccagaccagaccagaccagaccagaccagacctaa

SEQ ID NO: 23 - DNA, 1860 bp, Brachysprira hyodysenteriae
atgaatgataaatatgttatatgttcaattacaacaggatttacagatcaactcataaattttgctacagcaatatatattagagaaaaatataaaaaaaatgtaaaattagatatatcatggttttctgtttatgaaaatacagattatttgggaatagaaaaacgtaacctggaagtactaaatatatttaatgactttaattttgaaatagctacaaaaaaagaaatagatatagctttaaagtctatttttatagatggtagatatgaaaatatagataaaatattaaaatccaataaacatatatatgttggaccatatgttggacatactttattttactatggacatattaatatttcttcttttttcaatcttgacaaatattttattaatttattaaatgaaaaacataaaaacatattaaattacatgtcatcttttgaaagtgttgctatacatataagacgaggagattatatgtatgcgttaaaaatagatggaagaaagaataatgcttctttagactatatatatacatcaataaaatatttaaataatactttagaaaataaaaatataaaattttatatattttctaataataaaaaatatataaaaaataatttaataccaatttttcaaaataataatattaattttgaaattatagaagataatcctgaatatatagatttttatttaatgactaaatgtaagcatataatatcttcattaggaaaatttgcaaaagtagcttttaaatttataaaaaatgaaaataaaatatttatagatacagacaatatttataatttgaaaaaagaatatttctataaaaatatttgtgaaaaatactgcaacataatttatgaagaatataataatataaaaacatatatatcaaaaaataatatgatatatatactagacttaattgaaaaaaataatataaagaatatatgtcaaataggcttgctagacggtttagaaactcatactctattaaaatattcagcagacaataataactgttttaatttattatgttttgaaaataatgaaagagatatagttggaatagaattaaaaaatttaacagaaaatgaaaaaaaactatttacattatatatagaacagtctcctttagatatagataattcttttaatataaaaaatatagattttatcttttttacaaaagaaaattctaatcctctagtattcatttatttaatatacttatttccatttatgaaagaagaaatcataatagtatttaatgatattataagtggtaaatattattctggaagttattgtatatttaatatgtttaatggagaaaaaaatcaattttataattttaatacaaataaatatgacaatatcggatatataaaaataaagaaacaagaattattaaatataataaaaaaaatatctgttatagaatatgatggttatataaataaattttttatgaataaattaatagataacagaaatgattattataattattttgatttagatatgtcatacattaggttaaataatttaaagcaatatatgaaggataaatttgatatagaatattcaaattatataatctctaacatagaaaataatattattaattatgaaaaacaatattataaaaaccaaaaaaataatttttattctcaaaatcaaatacatatttctaaaatagaaaatataatagataaacataataaattaattaatagtatatcttggtggatacctataaaaaaattaagagaaaatttcaaaaataaatttataagaccagaccagaccagaccagaccagaccagaccagaccagacctaa

SEQ ID NO: 24 - DNA, 1860 bp, Brachysprira hyodysenteriae
atgaatgataaatatgttatatgttcaattacaacaggatttacagatcaactcataaattttgctacagcaatatatattagagaaaaatataaaaaaaatgtaaaattagatatatcatggttttctgtttatgaaaatacagattatttgggaatagaaaaacgtaacctggaagtactaaatatatttaatgactttaattttgaaatagctacaaaaaaagaaatagatatagctttaaagtctatttttatagatggtagatatgaaaatatagataaaatattaaaatccaataaacatatatatgttggaccatatgttggacatactttattttactatggacatattaatatttcttcttttttcaatcttgacaaatattttattaatttattaaatgaaaaacataaaaacatattaaattacatgtcatcttttgaaagtgttgctatacatataagacgaggagattatatgtatgcgttaaaaatagatggaagaaagaataatgcttctttagactatatatatacatcaataaaatatttaaataatactttagaaaataaaaatataaaattttatatattttctaataataaaaaatatataaaaaataatttaataccaatttttcaaaataataatattaattttgaaattatagaagataatcctgaatatatagatttttatttaatgactaaatgtaagcatataatatcttcattaggaaaatttgcaaaagtagcttttaaatttataaaaaatgaaaataaaatatttatagatacagacaatatttataatttgaaaaaagaatatttctataaaaatatttgtgaaaaatactgcaacataatttatgaagaatataataatataaaaacatatatatcaaaaaataatatgatatatatactagacttaattgaaaaaaataatataaagaatatatgtcaaataggcttgctagacggtttagaaactcatactctattaaaatattcagcagacaataataactgttttaatttattatgttttgaaaataatgaaagagatataattggaatagaattaaaaaatttaacagaaaatgaaaaaaaactatttacattatatatagaacagtctcctttagatatagataattcttttaatataaaaaatatagattttatcttttttacaaaagaaaattctaatcctctagtattcatttatttaatatacttatttccatttatgaaagaagaaatcataatagtatttaatgatattataagtggtaaatattattctggaagttattgtatatttaatatgtttaatggagaaaaaaatcaattttataattttaatacaaataaatatgacaatatcggatatataaaaataaagaaacaagaattattaaatataataaaaaaaatatctgttatagaatatgatggttatataaataaattttttatgaataaattaatagataacagaaatgattattataattattttgatttagatatgtcatacattaggttaaataatttaaagcaatatatgaaggataaatttgatatagaatattcaaattatataatctctaacatagaaaataatattattaattatgaaaaacaatattataaaaaccaaaaaaataatttttattctcaaaatcaaatacatatttctaaaatagaaaatataatagataaacataataaattaattaatagtatatcttggtggatacctataaaaaaattaagagaaaatttcaaaaataaatttataagaccagaccagaccagaccagaccagaccagaccagaccagacctaa

SEQ ID NO: 25 - DNA, 1857 bp, Brachysprira hyodysenteriae
atgaatgataaatatgttatatgttcaattacaacaggatttacagatcaactcataaattttgctacagcaatatatattagagaaaaatataaaaaaaatgtaaaattagatatatcatggttttctgtttatgaaaatacagattatttgggaatagaaaaacgtaacctggaagtactaaatatatttaatgactttaattttgaaatagctacaaaaaaagaaatagatatagctttaaagtctatttttatagatggtagatatgaaaatatagataaaatattaaaatccaataaacatatatatgttggaccatatgttggacatactttattttactatggacatattaatatttcttcttttttcaatcttgacaaatattttattaatttattaaatgaaaaacataaaaacatattaaattacatgtcatcttttgaaagtgttgctatacatataagacgaggagattatatgtatgcgttaaaaatagatggaagaaagaataatgcttctttagactatatatatacatcaataaaatatttaaataatactttagaaaataaaaatataaaattttatatattttctaataataaaaaatatataaaaaataatttaataccaatttttcaaaataataatattaattttgaaattatagaagataatcctgaatatatagatttttatttaatgactaaatgtaagcatataatatcttcattaggaaaatttgcaaaagtagcttttaaatttataaaaaatgaaaataaaatatttatagatacagacaatatttataatttgaaaaaagaatatttctataaaaatatttgtgaaaaatactgcaacataatttatgaagaatataataatataaaaacatatatatcaaaaaataatatgatatatatactagacttaattgaaaaaaataatataaagaatatatgtcaaataggcttgctagacggtttagaaactcatactctattaaaatattcagcagacaataataactgttttaatttattatgttttgaaaataatgaaagagatatagttggaatagaattaaaaaatttaacagaaaatgaaaaaaaactatttacattatatatagaacagtctcctttagatatagataattcttttaatataaaaaatatagattttatcttttttacaaaagaaaattctaatcctctagtattcatttatttaatatacttatttccatttatgaaagaagaaatcataatagtatttaatgatattataagtggtaaatattattctggaagttattgtatatttaatatgtttaatggagaaaaaaatcaattttataattttaatacaaataaatatgacaatatcggatatataaaaataaagaaacaagaattattaaatataataaaaaaaatatctgttatagaatatgatggttatataaataaattttttatgaataaattaatagataacagaaatgattattataattattttgatttagatatgtcatacattaggttaaataatttaaagcaatatatgaaggataaatttgatatagaatattcaaattatataatctctaacatagaaaataatattattaattatgaaaaacaatattataaaaaccaaaaaaataatttttattctcaaaatcaaatacatatttctaaaatagaaaatataatagataaacataataaattaattaatagtatatcttggtggatacctataaaagaattaagagaaaatttcaaaaataaatttataagaccagaccagaccagaccagaccagaccagacctaatatgtaa

SEQ ID NO: 26 - DNA, 1802 bp, Brachysprira hyodysenteriae
caatatatattagagaaaaatataaaaaaaatgtaaaattagatatatcatggttttctgtttatgaaaatacagattatttgggaatagaaaaacgtaacctggaagtactaaatatatttaatgactttaattttgaaatagctacaaaaaaagaaatagatatagctttaaagtctatttttatagatggtagatatgaaaatatagataaaatattaaaatccaataaacatatatatgttggaccatatgttggacatactttattttactatggacatattaatatttcttcttttttcaatcttgacaaatattttattaatttattaaataaaaaacataaaaacatattaaatgacatgtcatcttttgaaagtgttgctatacatataagacgaggagattatatgtatgcgttaaaaatagatggaagaaagaataatgcttctttagactatatatatacatcaataaaatatttaaataatactttagaaaataaaaatataaaattttatatattttctaataataaaaaatatataaaaaataatttaataccaatttttcaaaataataatattaattttgaaattatagaagataatcctgaatatatagatttttatttaatgactaaatgtaagcatataatatcttcattaggaaaatttgcaaaagtagcttttaaatttataaaaaatgaaaataaaatatttatagatacagacaatatttataatttgaaaaaagaatatttctataaaaatatttgtgaaaaatactgcaacataatttatgaagaatataataatataaaaacatatatatcaaaaaataatatgatatatatactagacttaattgaaaaaaataatataaagaatatatgtcaaataggcttgctagacggtttagaaactcatactctattaaaatattcagcatacaataataactgttttaatttattatgttttgaaaataatgaaagagatatagttggaatagaattaaaaaatttaacagaaaatgaaaaaaaactatttacattatatatagaacagtctcctttagatatagataattcttttaatataaaaaatatagattttatcttttttacaaaagaaaattctaatcctctagtattcatttatttaatatacttatttccatttatgaaagaagaaatcataatagtatttaatgatattataagtggtaaatattattctggaagttattgtatatttaatatgtttaatggagaaaaaaatcaattttataattttaatacaaataaatatgacaatatcggatatataaaaataaagaaacaagaattattaaatataataaaaaaaatatctgttatagaatatgatggttatataaataaattttttatgaataaattaatagataacagaaatgattattataattattttgatttagatatgtcatacattaggttaaataatttaaagcaatatatgaaggataaatttgatatagaatattcaaattatataatctctaacatagaaaataatattattaattatgaaaaacaatattataaaaaccaaaaaaataatttttattctcaaaatcaaatacatatttctaaaatagaaaatataatagataaacataataaattaattaatagtatatcttggtggatacctataaaaaaattaagagaaaatttcaaaaataaatttataagaccagaccagaccagaccagaccagaccagaccagaccagaccagacctaatatgtaa

SEQ ID NO: 27 - DNA, 1845 bp, Brachysprira hyodysenteriae
atgaatgataaatatgttatatgttcaattacaacaggatttacagatcaactcataaattttgctacagcaatatatattagagaaaaatataaaaaaaatgtaaaattagatatatcatggttttctgtttatgaaaatacagattatttgggaatagaaaaacgtaacctggaagtactaaatatatttaatgactttaattttgaaatagctacaaaaaaagaaatagatatagctttaaagtctatttttatagatggtagatatgaaaatatagataaaatattaaaatccaataaacatatatatgttggaccatatgttggacatactttattttactatggacatattaatatttcttcttttttcaatcttgacaaatattttattaatttattaaatgaaaaacataaaaacatattaaattacatgtcatcttttgaaagtgttgctatacatataagacgaggagattatatgtatgcgttaaaaatagatggaagaaagaataatgcttctttagactatatatatacatcaataaaatatttaaataatactttagaaaataaaaatataaaattttatatattttctaataataaaaaatatataaaaaataatttaataccaatttttcaaaataataatattaattttgaaattatagaagataatcctgaatatatagatttttatttaatgactaaatgtaagcatataatatcttcattaggaaaatttgcaaaagtagcttttaaatttataaaaaatgaaaataaaatatttatagatacagacaatatttataatttgaaaaaagaatatttctataaaaatatttgtgaaaaatactgcaacataatttatgaagaatataataatataaaaacatatatatcaaaaaataatatgatatatatactagacttaattgaaaaaaataatataaagaatatatgtcaaataggcttgctagacggtttagaaactcatactctattaaaatattcagcagacaataataactgttttaatttattatgttttgaaaataatgaaagagatatagttggaatagaattaaaaaatttaacagaaaatgaaaaaaaactatttacattatatatagaacagtctcctttagatatagataattcttttaatataaaaaatatagattttatcttttttacaaaagaaaattctaatcctctagtattcatttatttaatatacttatttccatttatgaaagaagaaatcataatagtatttaatgatattataagtggtaaatattattctggaagttattgtatatttaatatgtttaatggagaaaaaaatcaattttataattttaatacaaataaatatgacaatatcggatatataaaaataaagaaacaagaattattaaatataataaaaaaaatatctgttatagaatatgatggttatataaataaattttttatgaataaattaatagataacagaaatgattattataattattttgatttagatatgtcatacattaggttaaataatttaaagcaatatatgaaggataaatttgatatagaatattcaaattatataatctctaacatagaaaataatattattaattatgaaaaacaatattataaaaaccaaaaaaataatttttattctcaaaatcaaatacatatttctaaaatagaaaatataatagataaacataataaattaattaatagtatatcttggtggatacctataaaaaaattaagagaaaatttcaaaaataaatttataagaccagaccagaccagaccagaccagacctaa

SEQ ID NO: 28 - DNA, 1887 bp, Brachysprira hyodysenteriae
atgaatgataaatatgttatatgttcaattacaacaggatttacagatcaactcataaattttgctacagcaatatatattagagaaaaatataaaaaaaatgtaaaattagatatatcatggttttctgtttatgaaaatacagattatttgggaatagaaaaacgtaacctggaagtactaaatatatttaatgactttaattttgaaatagctacaaaaaaagaaatagatatagctttaaagtctatttttatagatggtagatatgaaaatatagataaaatattaaaatccaataaacatatatatgttggaccatatgttggacatactttattttactatggacatattaatatttcttcttttttcaatcttgacaaatattttattaatttattaaatgaaaaacataaaaacatattaaatgacatgtcatcttttgaaagtgttactatacatataagacgaggagattatatgtatgcgttaaaaatagatggaagaaagaataatgcttctttagactatatatatacatcaataaaatatttaaataatactttagaaaataaaaatataaaattttatatattttctaataataaaaaatatataaaaaataatttaataccaatttttcaaaataataatattaattttgaaattatagaagataatcctgaatatatagatttttatttaatgactaaatgtaagcatataatatcttcattaggaaaatttgcaaaagtagcttttaaatttataaaaaatgaaaataaaatatttatagatacagacaatatttataatttgaaaaaagaatatttctataaaaatatttgtgaaaaatactgcaacataatttatgaagaatataataatataaaaacatatatatcaaaaaataatatgatatatatactagacttaattgaaaaaaataatataaagaatatatgtcaaataggcttgctagacggtttagaaactcatactctattaaaatattcagcagacaataataactgttttaatttattatgttttgaaaataatgaaagagatatagttggaatagaattaaaaaatttaacagaaaatgaaaaaaaactatttacattatatatagaacagtctcctttagatatagataattcttttaatataaaaaatatagattttatcttttttacaaaagaaaattctaatcctctagtattcatttatttaatatacttatttccatttatgaaagaagaaatcataatagtatttaatgatattataagtggtaaatattattctggaagttattgtatatttaatatgtttaatggagaaaaaaatcaattttataattttaatacaaataaatatgacaatatcggatatataaaaataaagaaacaagaattattaaatataataaaaaaaatatctgttatagaatatgatggttatataaataaattttttatgaataaattaatagataacagaaatgattattataattattttgatttagatatgtcatacattaggttaaataatttaaagcaatatatgaaggataaatttgatatagaatattcaaattatataatctctaacatagaaaataatattattaattatgaaaaacaatattataaaaaccaaaaaaataatttttattctcaaaatcaaatacatatttctaaaatagaaaatataatagataaacataataaattaattaatagtatatcttggtggatacctataaaaaaattaagagaaaatttcaaaaataaatttataagaccagaccagaccagaccagaccagaccagaccagaccagaccagaccagaccagaccagacctaatatgtaa

SEQ ID NO: 29 - DNA, 1872 bp, Brachysprira hyodysenteriae
atgaatgataaatatgttatatgttcaattacaacaggatttacagatcaactcataaattttgctacagcaatatatattagagaaaaatataaaaaaaatgtaaaattagatatatcatggttttctgtttatgaaaatacagattatttgggaatagaaaaacgtaacctggaagtactaaatatatttaatgactttaattttgaaatagctacaaaaaaagaaatagatatagctttaaagtctatttttatagatggtagatatgaaaatatagataaaatattaaaatccaataaacatatatatgttggaccatatgttggacatactttattttactatggacatattaatatttcttcttttttcaatcttgacaaatattttattaatttattaaatgaaaaacataaaaacatattaaattacatgtcatcttttgaaagtgttgctatacatataagacgaggagattatatgtatgcgttaaaaatagatggaagaaagaataatgcttctttagactatatatatacatcaataaaatatttaaataatactttagaaaataaaaatataaaattttatatattttctaataataaaaaatatataaaaaataatttaataccaatttttcaaaataataatattaattttgaaattatagaagataatcctgaatatatagatttttatttaatgactaaatgtaagcatataatatcttcattaggaaaatttgcaaaagtagcttttaaatttataaaaaatgaaaataaaatatttatagatacagacaatatttataatttgaaaaaagaatatttctataaaaatatttgtgaaaaatactgcaacataatttatgaagaatataataatataaaaacatatatatcaaaaaataatatgatatatatactagacttaattgaaaaaaataatataaagaatatatgtcaaataggcttgctagacggtttagaaactcatactctattaaaatattcagcagacaataataactgttttaatttattatgttttgaaaataatgaaagagatatagttggaatagaattaaaaaatttaacagaaaatgaaaaaaaactatttacattatatatagaacagtctcctttagatatagataattcttttaatataaaaaatatagattttatcttttttacaaaagaaaattctaatcctctagtattcatttatttaatatacttatttccatttatgaaagaagaaatcataatagtatttaatgatattataagtggtaaatattattctggaagttattgtatatttaatatgtttaatggagaaaaaaatcaattttataattttaatacaaataaatatgacaatatcggatatataaaaataaagaaacaagaattattaaatataataaaaaaaatatctgttatagaatatgatggttatataaataaattttttatgaataaattaatagataacagaaatgattattataattattttgatttagatatgtcatacattaggttaaataatttaaagcaatatatgaaggataaatttgatatagaatattcaaattatataatctctaacatagaaaataatattattaattatgaaaaacaatattataaaaaccaaaaaaataatttttattctcaaaatcaaatacatatttctaaaatagaaaatataatagataaacataataaattaattaatagtatatcttggtggatacctataaaagaattaagagaaaatttcaaaaataaatttataagaccagaccagaccagaccagaccagaccagaccagaccagaccagacctaatatgtaa

SEQ ID NO: 30 - DNA, 1875 bp, Brachysprira hyodysenteriae
atgaatgataaatatgttatatgttcaattacaacaggatttacagatcaactcataaattttgctacagcaatatatattagagaaaaatataaaaaaaatgtaaaattagatatatcatggttttctgtttatgaaaatacagattatttgggaatagaaaaacgtaacctggaagtactaaatatatttaatgactttaattttgaaatagctacaaaaaaagaaatagatatagctttaaagtctatttttatagatggtagatatgaaaatatagataaaatattaaaatccaataaacatatatatgttggaccatatgttggacatactttattttactatggacatattaatatttcttcttttttcaatcttgacaaatattttattaatttattaaatgaaaaacataaaaacatattaaattacatgtcatcttttgaaagtgttgctatacatataagacgaggagattatatgtatgcgttaaaaatagatggaagaaagaataatgcttctttagactatatatatacatcaataaaatatttaaataatactttagaaaataaaaatataaaattttatatattttctaataataaaaaatatataaaaaataatttaataccaatttttcaaaataataatattaattttgaaattatagaagataatcctgaatatatagatttttatttaatgactaaatgtaagcatataatatcttcattaggaaaatttgcaaaagtagcttttaaatttataaaaaatgaaaataaaatatttatagatacagacaatatttataatttgaaaaaagaatatttctataaaaatatttgtgaaaaatactgcaacataatttatgaagaatataataatataaaaacatatatatcaaaaaataatatgatatatatactagacttaattgaaaaaaataatataaagaatatatgtcaaataggcttgctagacggtttagaaactcatactctattaaaatattcagcagacaataataactgttttaatttattatgttttgaaaataatgaaagagatatagttggaatagaattaaaaaatttaacagaaaatgaaaaaaaactatttacattatatatagaacagtctcctttagatatagataattcttttaatataaaaaatatagattttatcttttttacaaaagaaaattctaatcctctagtattcatttatttaatatacttatttccatttatgaaagaagaaatcataatagtatttaatgatattataagtggtaaatattattctggaagttattgtatatttaatatgtttaatggagaaaaaaatcaattttataattttaatacaaataaatatgacaatatcggatatataaaaataaagaaacaagaattattaaatataataaaaaaaatatctgttatagaatatgatggttatataaataaattttttatgaataaattaatagataacagaaatgattattataattattttgatttagatatgtcatacattaggttaaataatttaaagcaatatatgaaggataaatttgatatagaatattcaaattatataatctctaacatagaaaataatattattaattatgaaaaacaatattataaaaaccaaaaaaataatttttattctcaaaatcaaatacatatttctaaaatagaaaatataatagataaacataataaattaattaatagtatatcttggtggatacctataaaaaaattaagagaaaatttcaaaaataaatttataagaccagaccagaccagaccagaccagaccagaccagaccagaccagaccagaccagacctaa

SEQ ID NO: 31 - DNA, 1947 bp, Brachysprira hyodysenteriae
  misc_feature (1858)..(1899): n is a, c, g, or t
atgaatgataaatatgttatatgttcaattacaacaggatttacagatcaactcataaattttgctacagcaatatatattagagaaaaatataaaaaaaatgtaaaattagatatatcatggttttctgtttatgaaaatacagattatttgggaatagaaaaacgtaacctggaagtactaaatatatttaatgactttaattttgaaatagctacaaaaaaagaaatagatatagctttaaagtctatttttatagatggtagatatgaaaatatagataaaatattaaaatccaataaacatatatatgttggaccatatgttggacatactttattttactatggacatattaatatttcttcttttttcaatcttgacaaatattttattaatttattaaatgaaaaacataaaaacatattaaatgacatgtcatcttttgaaagtgttactatacatataagacgaggagattatatgtatgcgttaaaaatagatggaagaaagaataatgcttctttagactatatatatacatcaataaaatatttaaataatactttagaaaataaaaatataaaattttatatattttctaataataaaaaatatataaaaaataatttaataccaatttttcaaaataataatattaattttgaaattatagaagataatcctgaatatatagatttttatttaatgactaaatgtaagcatataatatcttcattaggaaaatttgcaaaagtagcttttaaatttataaaaaatgaaaataaaatatttatagatacagacaatatttataatttgaaaaaagaatatttctataaaaatatttgtgaaaaatactgcaacataatttatgaagaatataataatataaaaacatatatatcaaaaaataatatgatatatatactagacttaattgaaaaaaataatataaagaatatatgtcaaataggcttgctagacggtttagaaactcatactctattaaaatattcagcagacaataataactgttttaatttattatgttttgaaaataatgaaagagatatagttggaatagaattaaaaaatttaacagaaaatgaaaaaaaactatttacattatatatagaacagtctcctttagatatagataattcttttaatataaaaaatatagattttatcttttttacaaaagaaaattctaatcctctagtattcatttatttaatatacttatttccatttatgaaagaagaaatcataatagtatttaatgatattataagtggtaaatattattctggaagttattgtatatttaatatgtttaatggagaaaaaaatcaattttataattttaatacaaataaatatgacaatatcggatatataaaaataaagaaacaagaattattaaatataataaaaaaaatatctgttatagaatatgatggttatataaataaattttttatgaataaattaatagataacagaaatgattattataattattttgatttagatatgtcatacattaggttaaataatttaaagcaatatatgaaggataaatttgatatagaatattcaaattatataatctctaacatagaaaataatattattaattatgaaaaacaatattataaaaaccaaaaaaataatttttattctcaaaatcaaatacatatttctaaaatagaaaatataatagataaacataataaattaattaatagtatatcttggtggatacctataaaaaaattaagagaaaatttcaaaaataaatttataagaccagaccagaccagaccagaccagaccagaccagaccagaccnnnnnnnnnnnnnnnnnnnnnnnnnnnnnnnnnnnnnnnnnnagaccagaccagaccagaccagaccagaccagaccagaccagacctaa

SEQ ID NO: 32 - DNA, 864 bp, Brachysprira hyodysenteriae
atgaaaggaatcgtattagcaggcggaagcggaacaagactttatccaatgacaaatgttatatcaaaacagcttttgccaatatacgataaaccaatggtatattatccattatcagttttaatgctttcaaaaattaaagatatacttgtaatatctacagaaaatgatacaccgctttttaaaaaattattaaaagacggaagtcaatggggatgcaatattgaatatgctgtgcaaaaagcacctaatggacttgcagaagcatttataataggagaaagttttataggaaaagataatgtttctatgatattaggtgataatatattttttggacaaagcttttcttctgtattagaaaaaagtgctaagatagataatggggctgttatatttgcttatcaagtaaaagatcctgaacgtttcggtatagttgaaatagataaagactataatgctttatctatagaagaaaaacctaaaaatccaaagtctaattatgctgttacaggtctttatttttatgataatgatgtagttaatatttcaaaaaatattaagccttctgcaagaggggagcttgaaataacagatgttaataaagtatatttagaaaacaaaaaacttaaagttgaagtactaggacgcggatttgcttggcttgataccggtacaagagatagtcttcttcaggcttctcaatttgtgtctgctgtagaaaatattgaaggtatgcaaatagcttgtttagaagaaatagcttataataataaatggattgatgcttcaataatagaagagactgcatcaaaattgaaaaactcagaatatggtaaatacttgattaatttaataaaatag

SEQ ID NO: 33 - DNA, 864 bp, Brachysprira hyodysenteriae
atgaaaggaatcgtattagcaggcggaagcggaacaagactttatccaatgacaaatgttatatcaaaacagcttttgccaatatacgataaaccaatggtatattatccattatcagttttaatgctttcaaaaattaaagatatacttgtaatatctacagaaaatgatacaccgctttttaaaaaattattaaaagacggaagtcaatggggatgcaatattgaatatgctgtgcaaaaagcacctaatggacttgcagaagcatttataataggagaaagttttataggaaaagataatgtttctatgatattaggtgataatatattttttggacaaagcttttcttctgtattagaaaaaagtgctaagatatataatggggctgttatatttgcttatcaagtaaaagatcctgaacgtttcggtatagttgaaatagataaagattataatgctttatctatagaagaaaaacctaaaaatccaaagtctaattatgctgttacaggtctttatttttatgataatgatgtagttaatatttcaaaaaatattaagccttctgcaagaggggagcttgaaataacagatgttaataaagtatatttagaaaacaaaaaacttaaagttgaagtactaggacgcggatttgcttggcttgataccggtacaagagatagtcttcttcaggcttctcaatttgtatctgctgtagaaaatatcgaaggtatgcagatagcttgtttagaagaaatagcttataataataaatggattgatgcttcaaaaatagaagagactgcatcaaaattgaaaaactcagaatatggtaaatacttgattaatttaataaaataa

SEQ ID NO: 34 - DNA, 864 bp, Brachysprira hyodysenteriae
atgaaaggaatcgtattagcaggcggaagcggaacaagactttatccaatgacaaatgttatatcaaaacagcttttgccaatatacgataaaccaatggtatattatccattatcagttttaatgctttcaaaaattaaagatatacttgtaatatctacagaaaatgatacaccgctttttaaaaaattattaaaagacggaagtcaatggggatgcaatattgaatatgctgtgcaaaaagcacctaatggacttgcagaagcatttataataggagaaagttttataggaaaagataatgtttctatgatattaggtgataatatattttttggacaaagcttttcttctgtattagaaaaaagtgctaagatagataatggggctgttatatttgcttatcaagtaaaagatcctgaacgtttcggtatagttgaaatagataaagattataatgctttatctatagaagaaaaacctaaaaatccaaagtctaattatgctgttacaggtctttatttttatgataatgatgtagttaatatttcaaaaaatattaagccttctgcaagaggggagcttgaaataacagatgttaataaagtatatttagaaaacaaaaaacttaaagttgaagtactaggacgcggatttgcttggcttgataccggtacaagagatagtcttcttcaggcttctcaatttgtatctgctgtagaaaatatcgaaggtatgcagatagcttgtttagaagaaatagcttataataataaatggattgatgcttcaaaaatagaagagactgcatcaaaattgaaaaactcagaatatggtaaatacttgattaatttaataaaataa

SEQ ID NO: 35 - DNA, 864 bp, Brachysprira hyodysenteriae
atgaaaggaatcgtattagcaggcggaagcggaacaagactttatccaatgacaaatgttatatcaaaacagcttttgccaatatacgataaaccaatggtatattatccattatcagttttaatgctttcaaaaattaaagatatacttgtaatatctacagaaaatgatacaccgctttttaaaaaattattaaaagacggaaggcaatggggatgcaatattgaatatgctgtgcaaaaagcacctaatggacttgcagaagcatttataataggagaaagttttataggaaaagataatgtttctatgatattaggtgataatatattttttggacaaagcttttcttctgtattagaaaaaagtgctaagatagataatggggctgttatatttgcttatcaagtaaaagatcctgaacgtttcggtatagttgaaatagataaagattataatgctttatctatagaagaaaaacctaaaaatccaaagtctaattatgctgttacaggtctttatttttatgataatgatgtagttaatatttcaaaaaatattaagccttctgcaagaggggagcttgaaataacagatgttaataaagtatatttagaaaacaaaaaacttaaagttgaagtactaggacgcggatttgcttggcttgataccggtacaagagatagtcttcttcaggcttctcaatttgtatctgctgtagaaaatatcgaaggtatgcagatagcttgtttagaagaaatagcttataataataaatggattgatgcttcaaaaatagaagagactgcatcaaaattgaaaaactcagaatatggtaaatacttgattaatttaataaaataa

SEQ ID NO: 36 - DNA, 864 bp, Brachysprira hyodysenteriae
atgaaaggaatcgtattagcaggcggaagcggaacaagactttatccgatgacaaatgttatatcaaaacagcttttgccaatatacgataaaccgatggtatattatccattatcagttttaatgctttcaaaaattaaagatatacttgtaatatctacagaaaatgatacaccgctttttaaaaaattattaaaagacggaagtcaatggggatgcaatattgaatatgctgtgcaaaaagcacctaatggacttgcagaagcatttataataggagaaagttttataggaaaagataatgtttctatgatattaggtgataatatattttttggacaaagcttttcttctgtattagaaaaaagtgctaagatagataatggggctgttatatttgcttatcaagtaaaagatcctgaacgtttcggtatagttgaaatagataaagactataatgctttatctatagaagaaaaacctaaaaatccaaagtctaattatgctgttacaggtctttatttttatgataatgatgtagttaatatttcaaaaaatattaagccttctgcaagaggggagcttgaaataacagatgttaataaagtatatttagaaaacaaaaaacttaaagttgaagtactaggacgcggatttgcttggcttgataccggtacaagagatagtcttcttcaggcttctcaatttgtatctgctgtagaaaatatcgaaggtatgcagatagcttgtttagaagaaatagcttataataataaatggattgatgcttcaaaaatagaagagactgcatcaaaattgaaaaactcagaatatggtaaatacttaattaatttaattaaatag

SEQ ID NO: 37 - DNA, 864 bp, Brachysprira hyodysenteriae
atgaaaggaatcgtattagcaggcggaagcggaacaagactttatccaatgacaaatgttatatcaaaacagcttttgccaatatacgataaaccaatggtatattatccattatcagttttaatgctttcaaaaattaaagatatacttgtaatatctacagaaaatgatacaccgctttttaaaaaattattaaaagacggaaggcaatggggatgcaatattgaatatgctgtgcaaaaagcacctaatggacttgcagaagcatttataataggagaaagttttataggaaaagataatgtttctatgatattaggtgataatatattttttggacaaagcttttcttctgtattagaaaaaagtgctaagatagataatggggctgttatatttgcttatcaagtaaaagatcctgaacgtttcggtatagttgaaatagataaagactataatgctttatctatagaagaaaaacctaaaaatccaaagtctaattatgctgttacaggtctttatttttatgataatgatgtagttaatatttcaaaaaatattaagccttctgcaagaggggagcttgaaataacagatgttaataaagtatatttagaaaacaaaaaacttaaagttgaagtactaggacgcggatttgcttggcttgataccggtacaagagatagtcttcttcaggcttctcaatttgtatctgctgtagaaaatatcgaaggtatgcagatagcttgtttagaagaaatagcttataataataaatggattgatgcttcaaaaatagaagagactgcatcaaaattgaaaaactcagaatatggtaaatacttaattaatttaattaaatag

SEQ ID NO: 38 - DNA, 864 bp, Brachysprira hyodysenteriae
atgaaaggaatcgtattagcaggcggaagcggaacaagactttatccaatgacaaatgttatatcaaaacagcttttgccaatatacgataaaccaatggtatattatccattatcagttttaatgctttcaaaaattaaagatatacttgtaatatctacagaaaatgatacaccgctttttaaaaaattattaaaagacggaagtcaatggggatgcaatattgaatatgctgtgcaaaaagcacctaatggacttgcagaagcatttataataggagaaagttttataggaaaagataatgtttctatgatattaggtgataatatattttttggacaaagcttttcttctgtattagaaaaaagtgctaagatagataatggggctgttatatttgcttatcaagtaaaagatcctgaacgtttcggtatagttgaaatagataaagattataatgctttatctatagaagaaaaacctaaaaatccaaagtctaattatgctgttacaggtctttatttttatgataatgatgtagttaatatttcaaaaaatattaagccttctgcaagaggggagcttgaaataacagatgttaataaagtatatttagaaaacaaaaaacttaaagttgaagtactaggacgcggatttgcttggcttgataccggtacaagagatagtcttcttcaggcttctcaatttgtatctgctgtagaaaatatcgaaggtatgcagatagcttgtttagaagaaatagcttataataataaatggattgatgcttcaaaaatagaagagactgcatcaaaattgaaaaactcagaatatggtaaatacttaattaatttaattaaatag

SEQ ID NO: 39 - DNA, 864 bp, Brachysprira hyodysenteriae
atgaaaggaatcgtattagcaggcggaagcggaacaagactttatccaatgacaaatgttatatcaaaacagcttttgccaatatacgataaaccaatggtatattatccattatcagttttaatgctttcaaaaattaaagatatacttgtaatatctacagaaaatgatacaccgctttttaaaaaattattaaaagacggaagtcaatggggatgcaatattgaatatgctgtgcaaaaagcacctaatggacttgcagaagcatttataataggagaaagttttataggaaaagataatgtttctatgatattaggtgataatatattttttggacaaagcttttcttctgtattagaaaaaagtgctaagatagataatggggctgttatatttgcttatcaagtaaaagatcctgaacgtttcggtatagttgaaatagataaagactataatgctttatctatagaagaaaaacctaaaaatccaaagtctaattatgctgttacaggtctttatttttatgataatgatgtagttaatatttcaaaaaatattaagccttctgcaagaggggagcttgaaataacagatgttaataaagtatatttagaaaacaaaaaacttaaagttgaagtactaggacgcggatttgcttggcttgataccggtacaagagatagtcttcttcaggcttctcaatttgtatctgctgtagaaaatatcgaaggtatgcagatagcttgtttagaagaaatagcttataataataaatggattgatgcttcaaaaatagaagagactgcatcaaaattgaaaaactcagaatatggtaaatacttaattaatttaattaaatag

SEQ ID NO: 40 - DNA, 864 bp, Brachysprira hyodysenteriae
atgaaaggaatcgtattagcaggcggaagcggaacaagactttatccaatgacaaatgttatatcaaaacagcttttgccaatatacgataaaccaatggtatattatccattatcagttttaatgctttcaaaaattaaagatatacttgtaatatctacagaaaatgatacaccgctttttaaaaaattattaaaagacggaagtcaatggggatgcaatattgaatatgctgtgcaaaaagcacctaatggacttgcagaagcatttataataggagaaagttttataggaaaagataatgtttctatgatattaggtgataatatattttttggacaaagcttttcttctgtattagaaaaaagtgctaagatatataatggggctgttatatttgcttatcaagtaaaagatcctgaacgtttcggtatagttgaaatagataaagactataatgctttatctatagaagaaaaacctaaaaatccaaagtctaattatgctgttacaggtctttatttttatgataatgatgtagttaatatttcaaaaaatattaagccttctgcaagaggggagcttgaaataacagatgttaataaagtatatttagaaaacaaaaaacttaaagttgaagtactaggacgcggatttgcttggcttgataccggtacaagagatagtcttcttcaggcttctcaatttgtatctgctgtagaaaatatcgaaggtatgcagatagcttgtttagaagaaatagcttataataataaatggattgatgcttcaaaaatagaagagactgcatcaaaattgaaaaactcagaatatggtaaatacttaattaatttaattaaatag

SEQ ID NO: 41 - DNA, 864 bp, Brachysprira hyodysenteriae
atgaaaggaatcgtattagcaggcggaagcggaacaagactttatccaatgacaaatgttatatcaaaacagcttttgccaatatacgataaaccaatggtatattatccattatcagttttaatgctttcaaaaattaaagatatacttgtaatatctacagaaaatgatacaccgctttttaaaaaattattaaaagatggaagtcaatggggatgcaatattgaatatgctgtgcaaaaagcacctaatggacttgcagaagcatttataataggagaaagttttataggaaaagataatgtttctatgatattaggtgataatatattttttggacaaagcttttcttctgtattagaaaaaagtgctaagatagataatggggctgttatatttgcttatcaagtaaaagatcctgaacgtttcggtatagttgaaatagataaagactataatgctttatctatagaagaaaaacctaaaaatccaaagtctaattatgctgttacaggtctttatttttatgataatgatgtagttaatatttcaaaaaatattaagccttctgcaagaggggagcttgaaataacagatgttaataaagtatatttagaaaacaaaaaacttaaagttgaagtactaggacgcggatttgcttggcttgataccggtacaagagatagtcttcttcaggcttctcaatttgtatctgctgtagaaaatatcgaaggtatgcagatagcttgtttagaagaaatagcttataataataaatggattgatgcttcaaaaatagaagagactgcatcaaaattgaaaaactcagaatatggtaaatacttaattaatttaattaaatag

SEQ ID NO: 42 - DNA, 864 bp, Brachysprira hyodysenteriae
atgaaaggaatcgtattagcaggcggaagcggaacaagactttatccaatgacaaatgttatatcaaaacagcttttgccaatatacgataaaccaatggtatattatccattatcagttttaatgctttcaaaaattaaagatatacttgtaatatctacagaaaatgatacaccgctttttaaaaaattattaaaagatggaagtcaatggggatgcaatattgaatatgctgtacaaaaagcacctaatggacttgcagaagcatttataataggagaaagttttataggaaaagataatgtttctatgatattaggtgataatatattttttggacaaagcttttcttctgtattagaaaaaagtgctaagatagataatggggctgttatatttgcttatcaagtaaaagatcctgaacgtttcggtatagttgaaatagataaagactataatgctttatctatagaagaaaaacctaaaaatccaaagtctaattatgctgttacaggtctttatttttatgataatgatgtagttaatatttcaaaaaatattaagccttctgcaagaggggagcttgaaataacagatgttaataaagtatatttagaaaacaaaaaacttaaagttgaagtactaggacgcggatttgcttggcttgataccggtacaagagatagtcttcttcaggcttctcaatttgtatctgctgtagaaaatatcgaaggtatgcagatagcttgtttagaagaaatagcttataataataaatggattgatgcttcaaaaatagaagagactgcatcaaaattgaaaaactcagaatatggtaaatacttaattaatttaattaaatag

SEQ ID NO: 43 - DNA, 864 bp, Brachysprira hyodysenteriae
atgaaaggaatcgtattagcaggcggaagcggaacaagactttatccaatgacaaatgttatatcaaaacagcttttgccaatatacgataaaccaatggtatattatccattatcagttttaatgctttcaaaaattaaagatatacttgtaatatctacagaaaatgatacaccgctttttaaaaaattattaaaagacggaagtcaatggggatgcaatattgaatatgctgtacaaaaagcacctaatggacttgcagaagcatttataataggagaaagttttataggaaaagataatgtttctatgatattaggtgataatatattttttggacaaagcttttcttctgtattagaaaaaagtgctaagatagataatggggctgttatatttgcttatcaagtaaaagatcctgaacgtttcggtatagttgaaatagataaagactataatgctttatctatagaagaaaaacctaaaaatccaaagtctaattatgctgttacaggtctttatttttatgataatgatgtagttaatatttcaaaaaatattaagccttctgcaagaggggagcttgaaataacagatgttaataaagtatatttagaaaacaaaaaacttaaagttgaagtactaggacgcggatttgcttggcttgataccggtacaagagatagtcttcttcaggcttctcaatttgtatctgctgtagaaaatatcgaaggtatgcagatagcttgtttagaagaaatagcttataataataaatggattgatgcttcaaaaatagaagagactgcatcaaaattgaaaaactcagaatatggtaaatacttaattaatttaattaaatag

SEQ ID NO: 44 - DNA, 864 bp, Brachysprira hyodysenteriae
atgaaaggaatcgtattagcaggcggaagcggaacaagactttatccaatgacaaatgttatatcaaaacagcttttgccaatatacgataaaccaatggtatattatccattatcagttttaatgctttcaaaaattaaagatatacttgtaatatctacagaaaatgatacaccgctttttaaaaaattattaaaagacggaagtcaatggggatgcaatattgaatatgctgtgcaaaaagcacctaatggacttgcagaagcatttataataggagaaagttttataggaaaagataatgtttctatgacattaggtgataatatattttttggacaaagcttttcttctgtattagaaaaaagtgctaagatagataatggggctgttatatttgcttatcaagtaaaagatcctgaacgtttcggtatagttgaaatagataaagattataatgctttatctatagaagaaaaacctaaaaatccaaagtctaattatgctgttacaggtctttatttttatgataatgatgtagttaatatttcaaaaaatattaagccttctgcaagaggggagcttgaaataacagatgttaataaagtatatttagaaaacaaaaaacttaaagttgaagtactaggacgcggatttgcttggcttgataccggtacaagagatagtcttcttcaggcttctcaatttgtatctgctgtagaaaatatcgaaggtatgcagatagcttgtttagaagaaatagcttataataataaatggattgatgcttcaaaaatagaagagactgcatcaaaattgaaaaactcagaatatggtaaatacttgattaatttaataaaataa

SEQ ID NO: 45 - DNA, 1071 bp, Brachysprira hyodysenteriae
atgagaaattttaataatattttagttacaggcggatgcggttttattggaacaaattttataagatatatattgcaaaaaactgaatataaaggcaatattataaatatagatgccttaacttatgcaggaaacagagaaaatttaatagatatagaaaaaaaatataaagatagatatttctttgaaaaagttaatatttgtgatgacaaaaaagtaaatgaaatattttcacaatataatcctgattgtatagtacattttgcagcagaaagtcatgttgaccgttcaatatacggaccaaaagattttcttgaaacaaatataatggggacttttattttattagaagctgctcgtaaattatgggaaaacaataaagaaaataaactattccatcatataagcacagacgaagtttacggatctttaggtgaaacaggttatttttatgaaactacaccttatgatcctagaagtccttactcatcatcaaaagcttcaagcgatcatattgtaaaagcatattatcatacttataatctgcctgttacaatatctaattgcagcaacaattacggtccttatcaattccctgaaaaattaatacctttaatgatttctaatatagtagaagaaaaagatcttcctgtttatggtgatggaaaaaatataagagattggatatttgttgaagatcataataatgcagtaattgatataatacataaaggcagagtaggagagacttacaatatcggcggagaaaatgagattactaatattgatatggtaaatattttatgtgagaaattatctgctaaaatgaataaagaaaaagattattataaaaaattaatcaaatttgtaaaagacagatccggtcatgacagaagatatgctataaactgcgataaaataaaaaaagaattaggatgggaaagaaaatacgatttcaatacagccattgatataactatagattggtatttaaataacaaacaatggatagataatattaaaagcggagaatataaaaaatggattgagttaaactactcgtctaaataa

SEQ ID NO: 46 - DNA, 1071 bp, Brachysprira hyodysenteriae
atgagaaattttaataatattttagttactggcggatgcggttttataggatcaaactttataagatacatattagaaaaatctgattataaaggcaatattataaatatagatgccttaacttatgcaggaaacagagaaaatttaatagatatagaaaaaaaatataaagatagatatttctttgaaaaagttaatatttgtgatgataaaaaaataaatgaaatattttcaaaatataatcctgattgtatagtgcattttgctgcagaaagtcatgttgaccgttcaatatacggaccaaaagattttcttgaaactaatataatgggaacttttattttattagaagctgctcgtaaattatgggaaaataataaagaaaataaactattccatcatataagcacagacgaagtttacggatctttaggtgaaacaggttatttttatgaaactactccttatgatcctagaagtccttactcatcatcaaaagcttcaagcgatcatattgtaaaagcatattatcatacttataatctgcctgttacaatatctaattgcagcaacaattatggcccttatcaattccctgaaaaattaatacctttaatgatttctaatatagtagaagaaaaagatcttcctgtttatggagatggaaaaaatataagagattggatatttgttgaagatcataataatgcagtaattgatataatacataaaggcagggtaggagagacctacaatatcggcggagaaaacgagattactaatattgatatggtaaatattttatgtgagaaattatctgctaaaatgaataaagaaaaagattattataaaaaattaatcaaatttgtaaaagacagagctgggcatgacagaagatatgctataaactgcgataaaataaaaaaagaattaggatgggaaagaaaatacgatttcaatacagccattgatattactatagattggtatttaaataacaaacaatggatagataatattaaaagcggagaatataaaaaatggattgagttaaactactcgtctaaataa

SEQ ID NO: 47 - DNA, 1071 bp, Brachysprira hyodysenteriae
atgagaaattttaataatattttagttactggcggatgcggttttataggatcaaactttataagatacatattagaaaaatctgattataaaggcaatattataaatatagatgccttaacttatgcaggaaacagagaaaatttaatagatatagaaaaaaaatataaagatagatatttctttgaaaaagttaatatttgtgatgataaaaaaataaatgaaatattttcaaaatataatcctgattgtatagtgcattttgctgcagaaagtcatgttgaccgttcaatatacggaccaaaagattttcttgaaactaatataatgggaacttttattttattagaagctgctcgtaaattatgggaaaataataaagaaaataaactattccatcatataagcacagacgaagtttacggatctttaggtgaaacaggttatttttatgaaactactccttatgatcctagaagtccttactcatcatcaaaagcttcaagcgatcatattgtaaaagcatattatcatacttataatctgcctgttacaatatctaattgcagcaacaattatggcccttatcaattccctgaaaaattaatacctttaatgatttctaatatagtagaagaaaaagatcttcctgtttatggagatggaaaaaatataagagattggatatttgttgaagatcataataatgcagtaattgatataatacataaaggcagggtaggagagacttacaatattggcggagaaaacgagattactaatattgatatggtaaatattttatgtgagaaattatctgctaaaatgaataaagaaaaagattattataaaaaattaatcaaatttgtaaaagacagagccggtcatgacagaagatatgctataaactgcgataaaataaaaaaagaattaggatgggaaagaaaatacgatttcaataccgccattgatataactatagattggtatttaaataacaaacaatggatagataatattaaaagcggagaatataaaaaatggattgagttaaactactcgtctaaataa

SEQ ID NO: 48 - DNA, 1071 bp, Brachysprira hyodysenteriae
atgagaaattttaataatattttagttactggtggatgcggttttataggatcaaactttataagatacatattagaaaaatctgattataaaggcaatattataaatatagatgccttaacttatgcaggaaacagagaaaatttaatagatatagaaaaaaaatataaagatagatatttctttgaaaaagttaatatttgtgatgataaaaaaataaatgaaatattttcaaaatataatcctgattgtatagtgcattttgctgcagaaagtcatgttgaccgttcaatatacggaccaaaagattttcttgaaactaatataatgggaacttttattttattagaagctgctcgtaaattatgggaaaataataaagaaaataaactattccatcatataagcacagacgaagtttacggatctttaggtgaaacaggttatttttatgaaactactccttatgatcctagaagtccttactcatcatcaaaagcttcaagcgatcatattgtaaaagcatattatcatacttataatctgcctgttacaatatctaattgcagcaacaattatggcccttatcaattccctgaaaaattaatacctttaatgatttctaatatagtagaagaaaaagatcttcctgtttatggagatggaaaaaatataagagattggatatttgttgaagatcataataatgcagtaattgatataatacataaaggcagggtaggagagacttacaatattggcggagaaaacgagattactaatattgatatggtaaatattttatgtgagaaattatctgctaaaatgaataaagaaaaagattattataaaaaattaatcaaatttgtaaaagacagagccggtcatgacagaagatatgctataaactgcgataaaataaaaaaagaattaggatgggaaagaaaatacgatttcaatacagccattgatataactatagattggtatttaaataacaaacaatggatagataatattaaaagcggagaatataaaaaatggattgagttaaactactcatctaaataa

SEQ ID NO: 49 - DNA, 1071 bp, Brachysprira hyodysenteriae
atgagaaattttaataatattttagttactggcggatgcggttttataggatcaaactttataagatacatattagaaaaatctgattataaaggcaatattataaatatagatgccttaacttatgcaggaaacagagaaaatttaatagatatagaaaaaaaatataaagatagatatttctttgaaaaagttaatatttgtgatgataaaaaaataaatgaaatattttcaaaatataatcctgattgtatagtgcattttgctgcagaaagtcatgttgaccgttcaatatacggaccaaaagattttcttgaaactaatataatgggaacttttattttattagaagctgctcgtaaattatgggaaaataataaagaaaataaactattccatcatataagcacagacgaagtttacggatctttaggtgaaacaggttatttttatgaaactactccttatgatcctagaagtccttactcatcatcaaaagcttcaagcgatcatattgtaaaagcatattatcatacttataatctgcctgttacaatatctaattgcagcaacaattatggcccttatcaattccctgaaaaattaatacctttaatgatttctaatatagtagaagaaaaagatcttcctgtttatggagatggaaaaaatataagagattggatatttgttgaagatcataataatgcagtaattgatataatacataaaggcagggtaggagagacttacaatattggcggagaaaacgagattactaatattgatatggtaaatattttatgtgagaaattatctgctaaaatgaataaagaaaaagattattataaaaaattaatcaaatttgtaaaagacagagccggtcatgacagaagatatgctataaactgcgataaaataaaaaaagaattaggatgggaaagaaaatacgatttcaatacagccattgatataactatagattggtatttaaataacaaacaatggatagataatattaaaagcggagaatataaaaaatggattgagttaaactactcatctaaataa

SEQ ID NO: 50 - DNA, 1071 bp, Brachysprira hyodysenteriae
atgagaaattttaataatattttagttactggcggatgcggttttataggatcaaactttataagatacatattagaaaaatctgattataaaggcaatattataaatatagatgccttaacttatgcaggaaacagagaaaatttaatatatatagaaaaaaaatataaagatagatatttctttgaaaaagttaatatttgtgatgataaaaaaataaatgaaatattttcaaaatataatcctgattgtatagtgcattttgctgcagaaagtcatgttgaccgttcaatatacggaccaaaagattttcttgaaactaatataatgggaacttttattttattagaagctgctcgtaaattatgggaaaataataaagaaaataaactattccatcatataagcacagacgaagtttacggatctttaggtgaaacaggttatttttatgaaactactccttatgatcctagaagtccttactcatcatcaaaagcttcaagcgatcatattgtaaaagcatattatcatacttataatctgcctgttacaatatctaattgcagcaacaattatggcccttatcaattccctgaaaaattaatacctttaatgatttctaatatagtagaagaaaaagatcttcctgtttatggagatggaaaaaatataagagattggatatttgttgaagatcataataatgcagtaattgatataatacataaaggcagggtaggagagacttacaatattggcggagaaaacgagattactaatattgatatggtaaatattttatgtgagaaattatctgctaaaatgaataaagaaaaagattattataaaaaattaatcaaatttgtaaaagacagagccggtcatgacagaagatatgctataaactgcgataaaataaaaaaagaattaggatgggaaagaaaatacgatttcaatacagccattgatataactatagattggtatttaaataacaaacaatggatagataatattaaaagcggagaatataaaaaatggattgagttaaactactcatctaaataa

SEQ ID NO: 51 - DNA, 1071 bp, Brachysprira hyodysenteriae
atgagaaattttaataatattttagttactggcggatgcggttttataggctcaaactttataagatacatattagaaaaatctgattataaaggcaatattataaatatagatgccttaacttatgcaggaaacagagaaaatttaatagatatagaaaaaaaatataaagatagatatttctttgaaaaagttaatatttgtgatgataaaaaaataaatgaaatattttcaaaatataatcctgattgtatagtgcattttgctgcagaaagtcatgttgaccgttcaatatacggaccaaaagattttcttgaaactaatataatgggaacttttattttattagaagctgctcgtaaattatgggaaaataataaagaaaataaactattccatcatataagcacagacgaagtttacggatctttaggtgaaacaggttatttttatgaaactactccttatgatcctagaagtccttactcatcatcaaaagcttcaagtgatcatattgtaaaagcatattatcatacttataatctgcctgttacaatatctaattgcagcaacaattatggcccttatcaattccctgaaaaattaatacctttaatgatttctaatatagtagaagaaaaagatcttcctgtttatggagatggaaaaaatataagagattggatatttgttgaagatcataataatgcagtaattgatataatacataaaggcagggtaggagagacctacaatatcggcggagaaaacgagattactaatattgatatggtaaatattttatgtgagaaattatctgctaaaatgaataaagaaaaagattattataaaaaattaatcaaatttgtaaaagacagagccggtcatgacagaagatatgctataaactgcgataaaataaaaaaagaattaggatgggaaagaaaatacgatttcaatacaggcattgatattactatagattggtatttaaataacaaacaatggatagataatattaaaagcggagaatataaaaaatggattgagttaaactactcatctaaataa

SEQ ID NO: 52 - DNA, 1071 bp, Brachysprira hyodysenteriae
atgagaaattttaataatattttagttactggcggatgcggttttataggatcaaactttataagatacatattagaaaaatctgattataaaggcaatattataaatatagatgccttaacttatgcaggaaacagagaaaatttaatagatatagaaaaaaaatataaagatagatatttctttgaaaaagttaatatttgtgatgataaaaaaataaatgaaatattttcaaaatataatcctgattgtatagtgcattttgctgcagaaagtcatgttgaccgttcaatatacggaccaaaagattttcttgaaactaatataatgggaacttttattttattagaagctgctcgtaaattatgggaaaataataaagaaaataaactatttcatcatataagtacagacgaagtttacggatctttaggtgaaacaggttatttttatgaaactactccttatgatcctagaagtccttactcatcatcaaaagcttcaagtgatcatattgtaaaagcatattatcatacttataatctgcctgttacaatatctaattgcagcaacaattatggcccttatcaattccctgaaaaattaatacctttaatgatttctaatatagtagaagaaaaagatcttcctgtttatggagatggaaaaaatataagagattggatatttgttgaagatcataataatgcagtaattgatataatacataaaggcagggtaggagagacctacaatatcggcggagaaaacgagattactaatattgatatggtaaatattttatgtgagaaattatctgctaaaatgaataaagaaaaagattattataaaaaattaatcaaatttgtaaaagacagagccggtcatgacagaagatatgctataaactgcgataaaataaaaaaagaattaggatgggaaagaaaatacgatttcaatacagccattgatataactatagattggtatttaaataacaaacaatggatagataatattaaaagcggagaatataaaaaatggattgagttaaactactcgtctaaataa

SEQ ID NO: 53 - DNA, 1071 bp, Brachysprira hyodysenteriae
atgagaaattttaataatattttagttactggcggatgcggttttataggctcaaactttataagatacatattagaaaaatctgattataaaggcaatattataaatatagatgccttaacttatgcaggaaacagagaaaatttaatagatatagaaaaaaaatataaagatagatatttctttgaaaaagttaatatttgtgatgataaaaaaataaatgaaatattttcaaaatataatcctgattgtatagtgcattttgctgcagaaagtcatgttgaccgttcaatatacggaccaaaagattttcttgaaactaatataatgggaacttttattttattagaagctgctcgtaaattatgggaaaataataaagaaaataaactatttcatcatataagtacagacgaagtttacggatctttaggtgaaacaggttatttttatgaaactactccttatgatcctagaagtccttactcatcatcaaaagcttcaagtgatcatattgtaaaagcatattatcatacttataatctgcctgttacaatatctaattgcagcaacaattatggcccttatcaattccctgaaaaattaatacctttaatgatttctaatatagtagaagaaaaagatcttcctgtttatggagatggaaaaaatataagagattggatatttgttgaagatcataataatgcagtaattgatataatacataaaggcagggtaggagagacctacaatatcggcggagaaaacgagattactaatattgatatggtaaatattttatgtgagaaattatctgctaaaatgaataaagaaaaagattattataaaaaattaatcaaatttgtaaaagacagatccggtcatgacagaagatatgctataaactgcgataaaataaaaaaagaattaggatgggaaagaaaatacgatttcaatacagccattgatataactatagattggtatttaaataacaaacaatggatagataatattaaaagcggagaatataaaaaatggattgagttaaactactcgtctaaataa

SEQ ID NO: 54 - DNA, 1071 bp, Brachysprira hyodysenteriae
atgagaaattttaataatattttagttactggcggatgcggttttataggatcaaactttataagatacatattagaaaaatctgattataaaggcaatattataaatatagatgccttaacttatgcaggaaacagagaaaatttaatagatatagaaaaaaaatataaagatagatatttctttgaaaaagttaatatttgtgatgataaaaaaataaatgaaatattttcaaaatataatcctgattgtatagtgcattttgctgcagaaagtcatgttgaccgttcaatatacggaccaaaagattttcttgaaactaatataatgggaacttttattttattagaagctgctcgtaaattatgggaaaataataaagaaaataaactatttcatcatataagtacagacgaagtttacggatctttaggtgaaacaggttatttttatgaaactactccttatgatcctagaagtccttactcatcatcaaaagcttcaagtgatcatattgtaaaagcatattatcatacttataatctgcctgttacaatatctaattgcagcaacaattatggcccttatcaattccctgaaaaattaatacctttaatgatttctaatatagtagaagaaaaagatcttcctgtttatggagatggaaaaaatataagagattggatatttgttgaagatcataataatgcagtaattgatataatacataaaggcagggtaggagagacctacaatatcggcggagaaaacgagattactaatattgatatggtaaatattttatgtgagaaattatctgctaaaatgaataaagaaaaagattattataaaaaattaatcaaatttgtaaaagacagatccggtcatgacagaagatatgctataaactgcgataaaataaaaaaagaattaggatgggaaagaaaatacgatttcaatacagccattgatataactatagattggtatttaaataacaaacaatggatagataatattaaaagcggagaatataaaaaatggattgagttaaactactcgtctaaataa

SEQ ID NO: 55 - DNA, 579 bp, Brachysprira hyodysenteriae
atgccttttacatttaaaaaacttgatataaacggactttttatagcagaacctcgtatctttggagattcaagaggttatttcctagaaacatattctgagaaagatttttttgaagctggaatcaaagaaaaatttgtacaagataatcaatctaaatcaacaaaaaatgtattaagaggacttcattatcagaaaaaatattgtcaggctaaattggttagagtattagaaggtgaagtatttgatgttgcagtagatttgagaaatgattctccaacttttggaaaatattatggggtattattaacagaagaaaataaaaagcagttctttattcctaaaggttttgcacatggatttattgttttaagcgaatcagctgtttttgcttataaatgttctgacttttatcaccctgaagaagagggtggtataatatataatgataaaaatattaatatagattggacttgcgatactaaagatgtattagtatctgataaagatttaaaattactagagtttgacaaaaataaaaaatatttttcattagatggaaattggattggtgaataa

SEQ ID NO: 56 - DNA, 579 bp, Brachysprira hyodysenteriae
atgccttttacatttaaaaaacttgatataaacggactttttatagcagaacctcgtatctttggagattcaagaggttatttcctagaaacatattctgagaaagatttttttgaagctggaatcaaagaaaaatttgtacaagataatcaatctaaatcaacaaaaaatgtattaagaggacttcattatcagaaaaaatattgtcaggctaaattggttagagtattagaaggtgaagtatttgatgttgcagtagatttgagaaatgattctccaacttttggaaaatattatggggtattattaacagaagaaaataaaaagcagttctttattcctaaaggttttgcacatggatttattgttttaagcgaatcagctgtttttgcttataaatgttctgacttttatcaccctgaagaagagggtggtataatatataatgataaaaatattaatatagattggacttgcgatactaaagatgtattagtatctgataaagatttaaaattaccagagtttgacaaaaataaaaaatatttttcattagatggaaattggattggtgaataa

SEQ ID NO: 57 - DNA, 579 bp, Brachysprira hyodysenteriae
atgccttttacatttaaaaaacttgatataaacggactttttatagcagaacctcgtatctttggagattcaagaggttatttcctagaaacatattctgagaaagatttttttgaagctggaatcaaagaaaaatttgtacaagataatcaatctaaatcaacaaaaaatgtattaagaggacttcattatcagaaaaaatattgtcaggctaaattggttagagtattagaaggtaaagtatttgatgttgcagtagatttgagaaatgattctccaacttttggaaaatattatggtgttttattaacagaagaaaataaaaagcagttctttattcctaaaggttttgcacatggatttattgttctaagcgaatcagctgtttttgcttataaatgttctgacttttatcaccctgaagaagagggtggtataatatataatgataaaaatattaatatagattggacttgcgatactaaagatgtattagtatctgataaagatttaaaattaccagagtttgacaaaaataaaaaatatttttcattagatggaaattggattggagaataa

SEQ ID NO: 58 - DNA, 579 bp, Brachysprira hyodysenteriae
atgccttttacatttaaaaaacttgatataaacggactttttatagcagaacctcgtatctttggagattcaagaggttatttcctagaaacatattctgagaaagatttttttgaagctggaatcaaagaaaaatttgtacaagataatcaatctaaatcaacaaaaaatgtattaagaggacttcattatcagaaaaaatattgtcaggctaaattggttagagtattagaaggtgaagtatttgatgttgcagtagatttgagaaatgattctccaacttttggaaaatattatggtgttttattaacagaagaaaataaaaagcagttctttattcctaaaggttttgcacatggatttattgttctaagcgaatcagctgtttttgcttataaatgttctgacttttatcaccctgaagaagagggtggtataatatataatgataaaaatattaatatagattggacttgcgatactaaagatgtattagtatctgataaagatttaaaattaccagagtttgacaaaaataaaaaatatttttcattagatggaaattggattggagaataa

SEQ ID NO: 59 - DNA, 579 bp, Brachysprira hyodysenteriae
atgccttttacatttaaaaaacttgatataaacggactttttatagcagaacctcgtatctttggagattcaagaggttatttcctagaaacatattctgagaaagatttttttgaagctggaatcaaagaaaaatttgtacaagataatcaatctaaatcaacaaaaaatgtattaagaggacttcattatcagaaaaaatattgtcaggctaaattggttagagtattagaaggtgaagtatttgatgttgcagtagatttgagaaatgattctccaacttttggaaaatattatggggtattattaacagaagaaaataaaaagcagttctttattcctaaaggttttgcacatggatttattgttctaagcgaatcagctgtttttgcttataaatgttctgacttttatcaccctgaagaagagggtggtataatatataatgataaaaatattaatatagattggacttgcgatactaaagatgtattagtatctgataaagatttaaaattacctgagtttgacaaaaataaaaaatatttttcattagatggaaattggattggtgaataa

SEQ ID NO: 60 - DNA, 579 bp, Brachysprira hyodysenteriae
atgccttttacatttaaaaaacttgatataaacggactttttatagcagaacctcgtatctttggagattcaagaggttatttcctagaaacatattctgagaaagatttttttgaagctggaatcaaagaaaaatttgtacaagataatcaatctaaatcaacaaaaaatgtattaagaggacttcattatcagaaaaaatattgtcaggctaaattggttagagtattagaaggtgaagtatttgatgttgcagtagatttgagaaatgattctccaacttttggaaaatattatggggtattattaacagaagaaaataaaaagcagttctttattcctaaaggttttgcacatggatttattgttttaagcgaatcagctgtttttacttataaatgttctgacttttatcaccctgaagaagagggtggtataatatataatgataaaaatattaatatagattggacttgcgatactaaagatgtattagtatctgataaagatttaaaattaccagagtttgacaaaaataaaaaatatttttcattagatggaaattggattggagaataa

SEQ ID NO: 61 - DNA, 579 bp, Brachysprira hyodysenteriae; identical to SEQ ID NO: 60

SEQ ID NO: 62 - DNA, 861 bp, Brachysprira hyodysenteriae
atgatttgggtaattggtaaaaatggaatgcttgctcaagatatactcaaagcattcaataaaaataatatagaatatacttcaacagcttctaatatagatattactaatattgatatacttaataactttataaaagataaaaatattaaaacaataataaattgttctgcatatacaaaagtagatttagcagaagatgaaaaagatatttgctataaagttaatggagaaggtgtaaaaaatataacagaaatagcttctaatataaatgcagatttaattcatttttctactgattatgtatttgatggagaaaataacaaaccatacaatgaagaagataaaactaatccaataaatatttacggcaaaagtaaattagagggtgaaaaatatgctttattttataataaagcaatagttattagagtatcttggctatatggaatttatggaaaaaattttgtatatactatgattaatttgatgaatagtaaagaaagcataaaagtagttaatgatcaatttggatctcctacatatacagaagatgttgctatggtaatatatgatattataaaaaataacaattttgattatggactttatcattatactaatgaaggtaatatatcatggtatgattttgctaaaactatatataataaaggaaaagagtataatattataaataatgattgcaaagtaaatccatgtactacagaagaatatccaaccaaagctaaaagacctaaatacagtgttttatctgtagaaaaaataaaaagatatgttaaaatatataattatgaagatagcttaaatagtttttttaacatatatcaaaaaaataaataa

SEQ ID NO: 63 - DNA, 861 bp, Brachysprira hyodysenteriae
atgatttggataattggtaaaaatggaatgcttgctcaagatatactccaagtattcaataaaaataatatagaatatacttcaacagcttctaatatagatattactaatattgatatacttaataactttataaaagataaaaatattaaaacaataataaattgttctgcatatacaaaagtagatttagcagaagatgaaaaagatatttgctataaagttaatggagaaggtgtaaaaaatataacagaaatagcttctaatataaatgcagatttaattcatttttctactgattatgtatttgatggagaaaataacaaaccatacaatgaagaagataaaactaatccaataaatatttacggcaaaagtaaattagagggtgaaaaatatgctttatcctataataaatcaatagttattagagtatcttggctatatggtatttatggtaaaaattttgtatatactatgattaatttgatgaatagtaaagaaagcataaaagtagttaatgatcaatttggatctcctacatatacagaagatgttgctatggtaatatatgatattataaaaaataacaatttttattacggactttatcattatactaatgaaggtgatatatcatggtatgattttgctaaaactatatataataaaggaaaagagtataatattataaataatgattgcaaagtaaatccatgtactacagaagaatatccaaccaaagctaaaagacctaaatacagtgttttatctgtagaaaaaataaaaagatatgttaaaatatataattatgaagatagcttaaatagtttttttaacatatatcaaaaaaataaataa

SEQ ID NO: 64 - DNA, 861 bp, Brachysprira hyodysenteriae
atgatttggataattggtaaaaatggaatgcttgctcaagatatactccaagtattcaataaaaataatatagaatatacttcaacagcttctaatatagatattactaatattgatatacttaataactttataaaagataaaaatattaaaacaataataaattgttctgcatatacaaaagtagatttagcagaagatgaaaaagatatttgctataaagttaatggagaaggtgtaaaaaatataacagaaatagcttctaatataaatgcagatttaattcatttttctactgattatgtatttgatggagaaaataacaaaccatacaatgaagaagataaaactaatccaataaatatttacggcaaaagtaaattagagggtgaaaaatatgctttatcctataataaatcaatagttattagagtatcttggctatatggtatttatggtaaaaattttgtatatactatgattaatttgatgaatagtaaagaaagcataaaagtagttaatgatcaatttggatctcctacatatacagaagatgttgctatggtaatatatgatattataaaaaataacaattttgattacggactttatcattatactaatgaaggtgatatatcatggtatgattttgctaaaactatatataataaaggaaaagagtataatattataaataatgattgcaaagtaaatccatgtactacagaagaatatccaaccaaagctaaaagacctaaatacagtgttttatctgtagaaaaaataaaaagatatgttaaaatatataattatgaagatagcttaaatagtttttttaacatatatcaaaaaaataaataa

SEQ ID NO: 65 - DNA, 882 bp, Brachysprira hyodysenteriae
atgatttgggtaattggtaaaaatggaatgcttgctcaagatatactcaaagcattcaataaaaataatatagaatatacttcaacagcttctaatatagatattactaatattgatatacttaataactttataaaagataaaaatattaaaacaataataaattgttctgcatatacaaaagtagatttagcagaagatgaaaaagatatttgctataaagttaatggagaaggtgtaaaaaatataacagaaatagcttctaatataaatgcagatttaattcatttttctactgattatgtatttgatggagaaaataacaaaccatacaatgaagaagataaaactaatccaataaatatttacggcaaaagtaaattagagggtgaaaaatatgctttattttataataaagcaatagttattagagtatcttggctatatggaatttatggaaaaaattttgtatatactatgattaatttgatgaatagtaaagaaagcataaaagtagttaatgatcaatttggatctcctacatatacagaagatgttgctatggtaatatatgatattataaaaaataacaattttgattacggactttatcattatactaatgaaggtaatatatcatggtatgattttgctaaaactatatataataaaggaaaagagtataatattataaataatgattgcaaagtaaatccatgtactacagaagaatatccaaccaaagctaaaagacctaaatacagtgttttatctgtagaaaaaataaaaagatatgttaaaatatataattatgaagatagcttaaatagttttttaacatatatcaaaaaaataaataatttttggagtttattagtatga

SEQ ID NO: 66 - DNA, 861 bp, Brachysprira hyodysenteriae
atgatttggataattggtaaaaatggaatgcttgctcaagatatactccaagtattcaataaaaataatatagaatatacttcaacagcttctaatatagatattactaatattgatatacttaataactttataaaagataaaaatattaaaacaataataaattgttctgcatatacaaaagtagatttagcagaagatgaaaaagatatttgctataaagttaatggagaaggtgtaaaaaatataacagaaatagcttctaatataaatgcagatttaattcatttttctactgattatgtatttgatggagaaaataacaaaccatacaatgaagaagataaaactaatccaataaatatttacggcaaaagtaaattagagggtgaaaaatatgctttattttataataaagcaatagttattagagtatcttggctatatggaatttatggaaaaaattttgtatatactatgattaatttgatgaatagtaaagaaagcataaaagtagttaatgatcaatttggatctcctacatatacagaagatgttgctatggtaatatatgatattataaaaaataacaattttgattacggactttatcattatactaatgaaggtaatatatcatggtatgattttgctaaaactatatataataaaggaaaagagtataatattataaataatgattgcaaagtaaatccatgtactacagaagaatatccaaccaaagctaaaagacctaaatacagtgttttatctgtagaaaaaataaaaagatatgttaaaatatataattatgaagatagcttaaatagtttttttaacatatatcaaaaaaataaataa

SEQ ID NO: 67 - DNA, 861 bp, Brachysprira hyodysenteriae
atgatttgggtaattggtaaaaatggaatgcttgctcaagatatactcaaagcattcaataaaaataatatagaatatacttcaacagcttctaatatagatattactaatgttgatatacttaataactttataaaagataaaaatattaaaacaataataaattgttctgcatatacaaaagtagatttagcagaagatgaaaaagatatttgctataaagttaatggagaaggtgtaaaaaatataacagaaatagcttctaatataaatgcagatttaattcatttttctactgattatgtatttgatggagaaaataacaaaccatacaatgaagaagataaaactaatccaataaatatttacggcaaaagtaaattagagggtgaaaaatatgctttattttataataaagcaatagttattagagtatcttggctatatggaatttatggaaaaaattttgtatatactatgattaatttgatgaatagtaaagaaagcataaaagtagttaatgatcaatttggatctcctacatatacagaagatgttgctatggtaatatatgatattataaaaaataacaattttgattacggactttatcattatactaatgaaggtaatatatcatggtatgattttgctaaaactatatataataaaggaaaagagtataatattataaataatgattgcaaagtaaatccatgtactacagaagaatatccaaccaaagctaaaagacctaaatacagtgttttatctgtagaaaaaataaaaagatatgttaaaatatataattatgaagatagcttaaatagtttttttaacatatatcaaaaaaataaataa

SEQ ID NO: 68 - DNA, 861 bp, Brachysprira hyodysenteriae
atgatttgggtaattggtaaaaatggaatgcttgctcaagatatactcaaagcattcaataaaaataatatagaatatacttcaacagcttctaatatagatattactaatattgatatacttaataactttataaaagatcaaaatattaaaacaataataaattgttctgcatatacaaaagtagatttagcagaagatgaaaaagatatttgctataaagttaatggagaaggtgtaaaaaatataacagaaatagcttctaatataaatgcagatttaattcatttttctactgattatgtatttgatggagaaaataacaaaccatacaatgaagaagataaaactaatccaataaatatttacggcaaaagtaaattagagggtgaaaaatatgctttattttataataaagcaatagttattagagtatcttggctatatggaatttatggaaaaaattttgtatatactatgattaatttgatgaatagtaaagaaagcataaaagtagttaatgatcaatttggatctcctacatatacagaagatgttgctatggtaatatatgatattataaaaaataacaattttgattacggactttatcattatactaatgaaggtaatatatcatggtatgattttgctaaaactatatataataaaggaaaagagtataatattataaataatgattgcaaagtaaatccatgtactacagaagaatatccaaccaaagctaaaagacctaaatacagtgttttatctgtagaaaaaataaaaagatatgttaaaatatataattatgaagatagcttaaatagtttttttaacatatatcaaaaaaataaataa

SEQ ID NO: 69 - DNA, 981 bp, Brachysprira hyodysenteriae
atgaaaagattagccgtatttgcaggatatgataaagacaatataattgatgattatgttgtatattatattaaagaattaaaaaaaatagctgatataatctatgtatcagattgcaacatgtcagataatgaattaaaaaaaattaatgactactgcatacatatcataaatggaaggcatgaagaatatgatttcggatcatataaaagaggatatctatatgccaaagaaaataatatacttcaaaattatgattatttaatactttgtaatgattcttgctatggacctttatttgatttggaaaacataataaataaaatgaataattatgattttggaggtatatctattagtaaagatttaaaaatagcaaatcataattacataactagcttttttatatacataaataaaaacatatttaataaagatttctttaatgattttatttttaatataaaaaaagaagaagataaaatggatatcataaaaaaatatgagttcggactttcaaaactaatgcttgataataatatagaattaaaaggcttatttaatgataatggagagtttaacagaccatattttaatccattagccctaatagaagaaggatatccttttttaaaaaaacatgtactagaaaaaaaaataactgttcctttaaatataaatgaactaacaaatattataaaaataataaaaaataattacgatataaaattaatagttaatcatttaaacagagttgctgataaagaacaaattaaatatttatttcaaaaatataagccatataaaaaaatatttattcatgaaaaaattttctccttatttaccagatattcaccatcaggtaaatatcaaaccgtatataaattttttaattttctaactatatcaatagataaaccaattaaagattcatacatagaaactgactataaagattttaatttcttattagaaatataa

SEQ ID NO: 70 - DNA, 981 bp, Brachysprira hyodysenteriae
atgaaaagattagccgtatttgcaggatatgataaagacaatataattgatgattatgttgtatattatattaaagaattaaaaaaaatatctgatataatctatgtatcagattgcaatatgtcagataatgaattaaaaaaaattaatgattactgcatacatatcataaatggaaggcatgaagaatatgatttcggatcatataaaagaggatatctatatgccaaagaaaataatatacttcaaaattatgattatttaatactttgtaatgattcttgctatggacctttatttgatttggaaaacataataaataaaatgaatgattatgattttggaggcatatctattagtaaagatttaaaaatagcaaatcataattatataactagcttttttatatacataaacaaaaacatatttaataaagatttctttaatgattttatttatgatataaaaaaagaagaagataaaatggatatcataaaaaaatatgaatttggactttcaaaactaatgcttgataataatatagaattaaaaggcttatttaatgataatggagaatttaacagaccatactttaatccattagctctaatagaagaaggatatccttttttaaaaaaacatgtactagaaaaaaaagtaactgttcctttaaatatagatgaactaacaaatattataaaaataataaaaaataattacgatataaaattaatagttaatcatttaaatagagttgctgataaagaacaaattaaatatttatttcaaaaatataaaccatataaaaaaacatttattcatgaaaaaattttctccttatttaccagatattcaccatcaggtaaatatcaaacagtatataatttttttaattttctaactatatcaatagacaaaccaattaaagattcatacatagaaactgactataaagattttaattttttattagaaatataa

SEQ ID NO: 71 - DNA, 981 bp, Brachysprira hyodysenteriae
atgaaaagattagccgtatttgcaggatatgacaaagacaatataattgatgattatgttgtatattatattaaagaattaaaaaaaatatctgatataatctatgtatcagattgcaatatgtcagataatgaattaaaaaaaattaatgattactgcatacatatcataaatggaaggcatgaagaatatgatttcggatcatataaaagaggatatctatatgccaaagaaaataatatacttcaaaattatgattatttaatactttgtaatgattcttgctatggacctttatttgatttggaaaacataataaataaaatgaatgattatgattttggaggcatatctattagtaaagatttaaaaatagcaaatcataattatataactagcttttttatatacataaacaaaaacatatttaataaagatttctttaataattttatttatgatataaaaaaagaagaagataaaatggatatcataaaaaaatatgaatttggactttcaaaactaatgcttgataataatatagaattaaaaggcttatttaatgataatggagaatttaacagaccatactttaatccattagctctaatagaagaaggatatccttttttaaaaaaacatgtactagaaaaaaaagtaactgttcctttaaatatagatgaactaacaaatattataaaaataataaaaaataattacgatataaaattaatagttaatcatttaaatagagttgctgataaagaacaaattaaatatttatttcaaaaatataaaccatataaaaaaacatttattcatgaaaaaattttctccttatttaccagatattcaccatcaggtaaatatcaaacagtatataatttttttaattttctaactatatcaatagacaaaccaattaaagattcatacatagaaactgactataaagattttaattttttattagaaatataa

SEQ ID NO: 72 - DNA, 981 bp, Brachysprira hyodysenteriae
atgaaaagattagccgtatttgcaggatatgataaagacaatataattgatgattatgttgtatattatattaaagaattaaaaaaaatatctgatataatctatgtatcagattgcaatatgtcagataatgaattaaaaaaaattaatgattactgcatacatatcataaatggaaggcatgaagaatatgatttcggatcatataaaagaggatatctatatgccaaagaaaataatatacttcaaaattatgattatttaatactttgtaatgattcttgctatggacctttatttgatttggaaaacataataaataaaatgaatgattatgattttggaggcatatctattagtaaagatttaaaaatagcaaatcataattatataactagcttttttatatacataaacaaaaacatatttaataaagatttctttaatgattttatttatgatataaaaaaagaagaagataaaatggatatcataaaaaaatatgaatttggactttcaaaactaatgcttgataataatatagaattaaaaggcttatttaatgataatggagaatttaacagaccatactttaatccattagctctaatagaagaaggatatccttttttaaaaaaacatgtactagaaaaaaaagtaactgttcctttaaatatagatgaactaacaaatattataaaaataataaaaaataattacgatataaaattaatagttaatcatttaaatagagttgctgataaagaacaaattaaatatttatttcaaaaatataaaccatataaaaaaacatttattcatgaaaaaattttctccttatttaccagatattcaccatcaggtaaatatcaaaccgtatataaattttttaattttctaactatatcaatagacaaaccaattaaagattcatacatagaaactgactataaagattttaattttttattagaaatataa

SEQ ID NO: 73 - DNA, 981 bp, Brachysprira hyodysenteriae
atgaaaagattagccgtatttgcaggatatgacaaagacaatataattgatgattatgttgtatattatattaaagaattaaaaaaaatatctgatataatctatgtatcagattgcaatatgtcagataatgaattaaaaaaaattaatgattactgcatacatatcataaatggaaggcatgaagaatatgatttcggatcatataaaagaggatatctatatgccaaagaaaataatatacttcaaaattatgattatttaatactttgtaatgattcttgctatggacctttatttgatttggaaaacataataaataaaatgaatgattatgattttggaggcatatctattagtaaagatttaaaaatagcaaatcataattatataactagcttttttatatacataaacaaaaacatatttaataaagatttctttaatgattttatttatgatataaaaaaagaagaagataaaatggatatcataaaaaaatatgaatttggactttcaaaactaatgcttgataataatatagaattaaaaggcttatttaatgataatggagaatttaacagaccatactttaatccattagctctaatagaagaaggatatccttttttaaaaaaacatgtactagaaaaaaaagtaactgttcctttaaatatagatgaactaacaaatattataaaaataataaaaaataattacgatataaaattaatagttaatcatttaaatagagttgctgataaagaacaaattaaatatttatttcaaaaatataaaccatataaaaaaacatttattcatgaaaaaattttctccttatttaccagatattcaccatcaggtaaatatcaaacagtatataaattttttaattttctaactatatcaatagacaaaccaattaaagattcatacatagaaactgactataaagattttaattttttattagaaatataa

SEQ ID NO: 74 - DNA, 981 bp, Brachysprira hyodysenteriae
atgaaaagattagccgtatttgcaggatatgacaaagacaatataattgatgattatgttgtatattatattaaagaattaaaaaaaatatctgatataatctatgtatcagattgcaatatgtcagataatgaattaaaaaaaattaatgattactgcatacatatcataaatggaaggcatgaagaatatgatttcggatcatataaaagaggatatctatatgccaaagaaaataatatacttcaaaattatgattatttaatactttgtaatgattcttgctatggacctttatttgatttggaaaacataataaataaaatgaatgattatgattttggaggcatatctattagtaaagatttaaaaatagcaaatcataattatataactagcttttttatatacataaacaaaaacatatttaataaagatttctttaatgattttatttatgatataaaaaaagaagaagataaaatggatatcataaaaaaatatgaatttggactttcaaaactaatgcttgataataatatagaattaaaaggcttatttaatgataatggagaatttaacagaccatactttaatccattagctctaatagaagaaggatatccttttttaaaaaaacatgtactagaaaaaaaagtaactgttcctttaaatatagatgaactaacaaatattataaaaataataaaaaataattacgatataaaattaatagttaatcatttaaatagagttgctgataaagaacaaattaaatatttatttcaaaaatataaaccatataaaaaaacatttattcatgaaaaaattttctccttatttaccagatattcaccatcaggtaaatatcaaaccgtatataaattttttaattttctaactatatcaatagacaaaccaattaaagattcatacatagaaactgactataaagattttaattttttattagaaatataa

SEQ ID NO: 75 - DNA, 981 bp, Brachysprira hyodysenteriae
atgaaaagattagccgtatttgcaggatatgataaagacaatataattgatgattatgttgtatattatattaaagaattaaaaaaaatagctgatataatctatgtatcagattgcaatatgtcagataatgaattaaaaaaaattaataattactgcatacatatcataaatggaaggcatgaggaatatgattttggatcatataaaagaggatatctatatgccaaagaaaacaatatacttcaaaattatgattatttaatactttgtaatgattcttgctatggacctttatttgatttggaaaacatagtaaataaaatgaatgattatgatttcggaggtatatctattagtaaagatttaaaaatagcaaatcataattatataactagcttttttatatacataaacaaaaacatatttaataaagatttctttaatgattttatttatgatataaaaaaagaagaagataaaatggatatcataaaaaaatatgagtttggactttcaaaactcatgcttgataataatatagaattaaaaggcttatttaatgataatggagagtttaacaggccatattttaatccattagctctaatagaagaaggatatccttttttaaaaaaacatgtactagaaaaaaaagtaactgttcctttaaatatagatgaactaacaaatattataaaaataataaaaaataattacgatataaaattaatagttaatcatttaaatagagttgctgataaagaacaaattaaatatttatttcaaaaatataaaccatataaaaaaacatttattcatgaaaaaattttctccttatttaccagatattcaccatcaggtaaatatcaaacagtatataatttttttaattttctaactatatcaatagacaaaccaattaaagattcatacatagaaactgactataaagattttaattttttattagaaatataa

SEQ ID NO: 76 - DNA, 981 bp, Brachysprira hyodysenteriae
atgaaaagattagccgtatttgcaggatatgataaagacaatataattgatgattatgttgtatattatattaaagaattaaaaaaaatagctgatataatctatgtatcagattgcaatatgtcagataatgaattaaaaaaaattaataattactgcatacatatcataaatggaaggcatgaggaatatgattttggatcatataaaagaggatatctatatgccaaagaaaacaatatacttcaaaattatgattatttaatactttgtaatgattcttgctatggacctttatttgatttggaaaacatagtaaataaaatgaatgattatgatttcggaggtatatctattagtaaagatttaaaaatagcaaatcataattatataactagcttttttatatacataaacaaaaacatatttaataaagatttctttaatgattttatttatgatataaaaaaagaagaagataaaatggatatcataaaaaaatatgagtttggactttcaaaactcatgcttgataataatatagaattaaaaggcttatttaatgataatggagagtttaacaggccatattttaatccattagctctaatagaagaaggatatccttttttaaaaaaacatgtactagaaaaaaaagtaactgttcctttaaatatagatgaactaacaaatattataaaaataataaaaaataattacgatataaaattaatagttaatcatttaaatagagttgctgataaagaacaaattaaatatttatttcaaaaatataaaccatataaaaaaacatttattcatgaaaaaattttctccttatttaccagatattcaccatcaggtaaatatcaaacagtatataaattttttaattttctaactatatcaatagacaaaccaattaaagattcatacatagaaactgactataaagattttaatttcttattgaaaatataa

SEQ ID NO: 77 - DNA, 1188 bp, Brachysprira hyodysenteriae
atgaatacatgtgaatgcagtcaatgcaaaaatagagaatatgatatatcagaatatttagttcttgaagatgacttgaaaattttagataatgaaagaccactaggaatatcaggacatttaagggttaaagatgaagctatgagtttagccgaatgtattgattcatgcatagactttttagatgaacttataataacttataacacatcttcagataacacagaagaaatattaaaaaactacgaaaaaaaatatcctgaaaaaattaaactatttcattataaacctaatattattagatacaataaagatgagtataatacaaaatattcacaaatacactatttagataattattacaattatggactaattaaaataaaatacaaatattatgttaaaatagatgctgatcaaatttatttcacagataaattattaaaattaagagaattattaatattagatataaatcaaataaaattaagcaataatataaataaattattattaataaataaaatagcttggtgcatacctatcaaaaaattacgaaataaatttagaacatattgtattagaaagtatttcaataaagataaaaattatatttataatggtgatgaaaatatattatcattagatgattttattttattatctaggaaaagacattcagataaaaatatcagttatgatcttgcaggattcaatctaatattacataataataaattatatttagatacatattggcctatgaatggcggacgttttgatcatgtgctgtatcaagcatcttattctaataaatattttatgaatgaaatatttgaagtaaatcaaactcctcataatagaataattttagggatgttttgggtacattttggaaatattaaacataattttcaatatgatgattctttttatttactagaatataatgatataaaaaattctaaagtagaaaatttttctcataaaatgaaaaatattgataatgatgataaaatagaaaaatatataagagataatacttttataaaatattttgatgaggataaaaaataccttaataatgttttttataataaatacttgacaaaaccacttgactatgctataaaaaacaatgatgaattagtaaatttatatggaaaaactattattactgtaaaatga

SEQ ID NO: 78 - DNA, 1218 bp, Brachysprira hyodysenteriae
atgataagttatttaaataaaaggtataaaatgaataaatgtgaatgcagtcaatgcaaaaatagagaatatgatatatcagaatatttggttcttgaagatgacttgaaaattttagataatgaaagaccactaggaatatcaggacatttaagggttaaagatgaagctatgagtttagctgaatgcattgattcatgcatagactttttagatgaacttataataacttataacacatcttcagataacacagaagaaatattaaaaaactacgaaaaaaaatatcctgaaaaaattaaactatttcattataaacctaatattataagatacaataaagatgagtataatacaaaatattcacaaatacactatttagataattattacaattatggactaattaaaataaaatacaaatattatgttaaaatagatgctgatcaaatttatttcacagataaattattaaaattaagagaattattaatattagatataaatcaaataaaattaagcaataatataaataaattattattaataaataaaatagcttggtgcatacctatcaaaaaattacgaaataaatttagaacatattgtattagaaagtatttcaataaagataaaaattatatttataatggtgatgaaaatatattatcattagatgatttcattttattatctaggaaaagacattcagataaaaatatcagttatgatcttgcaggattcaatctaatattacataataataaattatatttagatatatattggcctatgaatggcggacgttttgatcatgtgctgtatcaagcatcttattctaataaatattttatgaatgaaatatttgaagtaaatcaaactcctcataatagaataattttagggatgttttgggtacattttggaaatattaaacataattttcaatatgatgattctttttatttactagaatataatgatataaaaaattctaaagtagaaaatttttctcataaaatgaaaaatattgataatgatgataaaatagaaaaatatataagagataatacttttataaaatattttgatgaggataaaaaataccttaataatgttttttataataaatacttgacaaaaccacttgactatgctataaaaaacaatgatgaattagtaaatttatatggaaaaactattattactgtaaaatga

SEQ ID NO: 79 - DNA, 1188 bp, Brachysprira hyodysenteriae
atgaataaatgtgaatgcagtcaatgccaaaatagagaatatgatatatcagaatatttggttcttgaagatgacttgaaaattttagataatgaaagaccactaggaatatcaggacatttaagagttaaagatgaagctatgagtttagctgaatgcattgattcatgcatagactttttagatgaacttataataacttataacacatcttcagataacacagaagaaatattaaaaaactacgaaaaaaaatatcctgaaaaaattaaactatttcattataaacctaatattataagatacaataaagatgagtataatacaaaatattcacaaatacactatttagataattattacaattatggactaattaaaataaaatacaaatattatgttaaaatagatgctgatcaaatttatttcacagataaattattaaaattaagagaattattaatattagatataaatcaaataaaattaagcaataatataaataaattattattaataaataaaatagcttggtgcatacctatcaaaaaattacgaaataaatttagaacatattgtattagaaagtatttcaataaagataaaaattatatttataatggtgatgaaaatatattatcattagatgattttattttattatctaggaaaagacattcagataaaaatatcagttatgatcttgcaggattcaatctaatattacataataataaattatatttagatacatattggcctatgaatggcggacgttttgatcatgtgctgtatcaagcatcttattctaataaatattttatgaatgaaatatttgaagtaaatcaaactcctcataatagaataattttagggatgttttgggtacattttggaaatattaaacataattttcaatatgatgattctttttatttactagaatataatgatataaaaaattctaaagtagaaaatttttctcataaaatgaaaaatattgataatgatgataaaatagaaaaatatataagagataatacttttataaaatattttgatgaggataaaaaataccttaataatgttttttataataaatacttgacaaaaccacttgactatgctataaaaaacaatgatgaattagtaaatttatatggaaaaactattattactgtaaaatga

SEQ ID NO: 80 - DNA, 1218 bp, Brachysprira hyodysenteriae
atgataagttatttaaataaaaggtataaaatgaataaatgtgaatgcagtcaatgcaaaaatagagaatatgatatatcagaatatttggttcttgaagatgacttgaaaattttagataatgaaagaccactaggaatatcaggacatttaagagttaaagatgaagctatgagtttagctgaatgcattgattcatgcatagactttttagatgaacttataataacttataacacatcttcagataacacagaagaaatattaaaaaactacgaaaaaaaatatcctgaaaaaattaaactatttcattataaacctaatattataagatacaataaagatgagtataatacaaaatattcacaaatacactatttagataattattacaattatggactaattaaaataaaatacaaatattatgttaaaatagatgctgatcaaatttatttcacagataaattattaaaattaagagaattattaatattagatataaatcaaataaaattaagcaataatataaataaattattattaataaataaaatagcttggtgcatacctatcaaaaaattacgaaataaatttagaacatattgtattagaaagtatttcaataaagataaaaattatatttataatggtgatgaaaatatattatcattagatgattttattttattatctaggaaaagacattcagataaaaatatcagttatgatcttgcaggattcaatctaatattacataataataaattatatttagatacatattggcctatgaatggcggacgttttgatcatgtgctgtatcaagcatcttattctaataaatattttatgaatgaaatatttgaagtaaatcaaactcctcataatagaataattttagggatgttttgggtacattttggaaatattaaacataattttcaatatgatgattctttttatttactagaatataatgatataaaaaattctaaagtagaaaatttttctcataaaatgaaaaatattgataatgatgataaaatagaaaaatatataagagataatacttttataaaatattttgatgaggataaaaaataccttaataatgttttttataataaatacttgacaaaaccacttgactatgctataaaaaacaatgatgaattagtaaatttatatggaaaaactattattactgtaaaatga

SEQ ID NO: 81 - DNA, 1188 bp, Brachysprira hyodysenteriae
atgaataaatgtgaatgcagtcaatgcaaaaatagagaatatgatataccagaatatttggttcttgaagatgacttgaaaattttagataatgaaagaccactaggaatatcaggacatttaagggttaaagatgaagctatgagtttagctgaatgcattgattcatgcatagactttttagatgaacttataataacttataacacatcttcagataacacagaagaaatattaaaaaactacgaaaaaaaatatcctgaaaaaattaaactatttcattataaacctaatattataagatacaataaagatgagtataatacaaaatattcacaaatacactatttagataattattacaattatggactaagtaaaataaaatacaaatattatgttaaaatagatgctgatcaaatttatttcacagataaattattaaaattaagagaattattaatattagatataaatcaaataaaattaagcaataatataaataaattattattaataaataaaatagcttggtgcatacctatcaaaaaattacgaaataaatttagaacatattgtattagaaagtatttcaataaagataaaaattatatttataatggtgatgaaaatatattatcattagatgattttattttattatctaggaaaagacattcagataaaaatatcagttatgatcttgcaggattcaatctaatattacataataataaattatatttagatacatattggcctatgaatggcggacgttttgatcatgtgctgtatcaagcatcttattctaataaatattttatgaatgaaatatttgaagtaaatcaaactcctcataatagaataattttagggatgttttgggtacattttggaaatattaaacataattttcaatatgatgattctttttatttactagaatataatgatataaaaaattctaaagtagaaaatttttctcataaaatgaaaaatattgataatgatgataaaatagaaaaatatataagagataatacttttataaaatattttgatgaggataaaaaataccttaataatgttttttataataaatacttgacaaaaccacttgactatgctataaaaaacaatgatgaattagtaaatttatatggaaaaactattattactgtaaaatga

SEQ ID NO: 82 - DNA, 795 bp, Brachysprira hyodysenteriae
atgtctagcaatataatagatacaatagcttggtggattcctattaaatccattagaataaaatttagaaatgcaacaaagaaaaagttcaataattttatttttaacaaaatagattttaactacgttaatacaaataatatagaacttatactcaataaatacaatgaaatatttgtcccattaatattaagtaacgaaaaaaaatacaaaaaaaatagaaatagcgagttaataaataaatatttaactggtttaaaaggtgttgaaataggaggaagcgctggagctgattttggtatagacgctatacaagttaatattgtcgatgccaatcaggaacacaataattattatcaacaaacaaatattttaggttcgggatataatcaaagaactgatatcatatcatgtggtgatgtattaccatttcaagataattcaatagactttgtattctcaagtcatgtactagaacatttctatgaccctataaaagctttaaaagaatggtacagagttataaaaaaagacggatatatagctatgatagtacctcataaagaaagaacattcgataaagatagagatagaacaactttaaatgaattaatagaaagacataataaggttaaaaattataaatacaatgtggatgaaagaggaatgcatttttctgtttggataacagaagatatactagaattatgcaaatatttaaatatgaatgtaattgaatatctagatgtagatgatgctgtaggtaatggatttgctattatcataaaaaaataa

SEQ ID NO: 83 - protein, 648 aa, Brachysprira hyodysenteriae
  misc_feature (620)..(633): Xaa can be any naturally occurring amino acid
MNDKYVICSITTGFTDQLINFATAIYIREKYKKNVKLDISWFSVYENTDYLGIEKRNLEVLNIFNDFNFEIATKKEIDIALKSIFIDGRYENIDKILKSNKHIYVGPYVGHTLFYYGHINISSFFNLDKYFINLLNEKHKNILNDMSSFESVTIHIRRGDYMYALKIDGRKNNASLDYIYTSIKYLNNTLENKNIKFYIFSNNKKYIKNNLIPIFQNNNINFEIIEDNPEYIDFYLMTKCKHIISSLGKFAKVAFKFIKNENKIFIDTDNIYNLKKEYFYKNICEKYCNIIYEEYNNIKTYISKNNMIYILDLIEKNNIKNICQIGLLDGLETHTLLKYSADNNNCFNLLCFENNERDIVGIELKNLTENEKKLFTLYIEQSPLDIDNSFNIKNIDFIFFTKENSNPLVFIYLIYLFPFMKEEIIIVFNDIISGKYYSGSYCIFNMFNGEKNQFYNFNTNKYDNIGYIKIKKQELLNIIKKISVIEYDGYINKFFMNKLIDNRNDYYNYFDLDMSYIRLNNLKQYMKDKFDIEYSNYIISNIENNIINYEKQYYKNQKNNFYSQNQIHISKIENIIDKHNKLINSISWWIPIKKLRENFKNKFIRPDQTRPDQTRPDQTXXXXXXXXXXXXXXRPDQTRPDQTRPDQT

SEQ ID NO: 84 - protein, 624 aa, Brachysprira hyodysenteriae
MNDKYVICSITTGFTDQLINFATAIYIREKYKKNVKLDISWFSVYENTDYLGIEKRNLEVLNIFNDFNFEIATKKEIDIALKSIFIDGRYENIDKILKSNKHIYVGPYVGHTLFYYGHINISSFFNLDKYFINLLNEKHKNILNYMSSFESVAIHIRRGDYMYALKIDGRKNNASLDYIYTSIKYLNNTLENKNIKFYIFSNNKKYIKNNLIPIFQNNNINFEIIEDNPEYIDFYLMTKCKHIISSLGKFAKVAFKFIKNENKIFIDTDNIYNLKKEYFYKNICEKYCNIIYEEYNNIKTYISKNNMIYILDLIEKNNIKNICQIGLLDGLETHTLLKYSADNNNCFNLLCFENNERDIVGIELKNLTENEKKLFTLYIEQSPLDIDNSFNIKNIDFIFFTKENSNPLVFIYLIYLFPFMKEEIIIVFNDIISGKYYSGSYCIFNMFNGEKNQFYNFNTNKYDNIGYIKIKKQELLNIIKKISVIEYDGYINKFFMNKLIDNRNDYYNYFDLDMSYIRLNNLKQYMKDKFDIEYSNYIISNIENNIINYEKQYYKNQKNNFYSQNQIHISKIENIIDKHNKLINSISWWIPIKKLRENFKNKFIRPDQTRPDQTRPDQTRPDQT

SEQ ID NO: 85 - protein, 605 aa, Brachysprira hyodysenteriae
MNDKYVICSITTGFTDQLINFATAIYIREKYKKNVKLDISWFSVYENTDYLGIEKRNLEVLNIFNDFNFEIATKKEIDIALKSIFIDGRYENIDKILKSNKHIYVGHTLFYYGHINISSFFNLDKYFINLLNEKHKNILNYMSSFESVAIHIRRGDYMYALKIDGRKNNASLDYIYTSIKYLNNTLENKNIKFYIFSNNKKYIKNNLIPIFQNNNINFEIIEDNPEYIDFYLMTKCKHIISSLGKFAKVAFKFIKNENKIFIDTDNIYNLKKEYFYKNICEKYCNIIYEEYNNIKTYISKNNMIYILDLIEKNNIKNICQIGLLDGLETHTLLKYSADNNNCFNLLCFENNERDIVGIELKNLTENEKKLFTLYIEQSPLDIDNSFNIKNIDFIFFTKENSNPLVFIYLIYLFPFMKEEIIIVFNDIISGKYYSGSYCIFNMFNGEKNQFYNFNTNKYDNIGYIKIKKQELLNIIKKISVIEYDGYINKFFMNKLIDNRNDYYNYFDLDMSYIRLNNLKQYMKDKFDIEYSNYIISNIENNIINYEKQYYKNQKNNFYSQNQIHISKIENIIDKHNKLINSISWWIPIKELRENFKNKFIRPDQT

SEQ ID NO: 86 - protein, 628 aa, Brachysprira hyodysenteriae
MNDKYVICSITTGFTDQLINFATAIYIREKYKKNVKLDISWFSVYENTDYLGIEKRNLEVLNIFNDFNFEIATKKEIDIALKSIFIDGRYENIDKILKSNKHIYVGPYVGHTLFYYGHINISSFFNLDKYFINLLNEKHKNILNDMSSFESVTIHIRRGDYMYALKIDGRKNNASLDYIYTSIKYLNNTLENKNIKFYIFSNNKKYIKNNLIPIFQNNNINFEIIEDNPEYIDFYLMTKCKHIISSLGKFAKVAFKFIKNENKIFIDTDNIYNLKKEYFYKNICEKYCNIIYEEYNNIKTYISKNNMIYILDLIEKNNIKNICQIGLLDGLETHTLLKYSADNNNCFNLLCFENNERDIVGIELKNLTENEKKLFTLYIEQSPLDIDNSFNIKNIDFIFFTKENSNPLVFIYLIYLFPFMKEEIIIVFNDIISGKYYSGSYCIFNMFNGEKNQFYNFNTNKYDNIGYIKIKKQELLNIIKKISVIEYDGYINKFFMNKLIDNRNDYYNYFDLDMSYIRLNNLKQYMKDKFDIEYSNYIISNIENNIINYEKQYYKNQKNNFYSQNQIHISKIENIIDKHNKLINSISWWIPIKKLRENFKNKFIRPDQTRPDQTRPDQTRPDQTRPNM

SEQ ID NO: 87 - protein, 639 aa, Brachysprira hyodysenteriae
MNDKYVICSITTGFTDQLINFATAIYIREKYKKNVKLDISWFSVYENTDYLGIEKRNLEVLNIFNDFNFEIATKKEIDIALKSIFIDGRYENIDKILKSNKHIYVGPYVGHTLFYYGHINISSFFNLDKYFINLLNEKHKNILNYMSSFESVAIHIRRGDYMYALKIDGRKNNASLDYIYTSIKYLNNTLENKNIKFYIFSNNKKYIKNNLIPIFQNNNINFEIIEDNPEYIDFYLMTKCKHIISSLGKFAKVAFKFIKNENKIFIDTDNIYNLKKEYFYKNICEKYCNIIYEEYNNIKTYISKNNMIYILDLIEKNNIKNICQIGLLDGLETHTLLKYSADNNNCFNLLCFENNERDIIGIELKNLTENEKKLFTLYIEQSPLDIDNSFNIKNIDFIFFTKENSNPLVFIYLIYLFPFMKEEIIIVFNDIISGKYYSGSYCIFNMFNGEKNQFYNFNTNKYDNIGYIKIKKQELLNIIKKISVIEYDGYINKFFMNKLIDNRNDYYNYFDLDMSYIRLNNLKQYMKDKFDIEYSNYIISNIENNIINYEKQYYKNQKNNFYSQNQIHISKIENIIDKHNKLINSISWWIPIKKLRENFKNKFIRPDQTRPDQTRPDLICKEYIYNILYYKINKLQYIN

SEQ ID NO: 88 - protein, 644 aa, Brachysprira hyodysenteriae
MNDKYVICSITTGFTDQLINFATAIYIREKYKKNVKLDISWFSVYENTDYLGIEKRNLEVLNIFNDFNFEIATKKEIDIALKSIFIDGRYENIDKILKSNKHIYVGPYVGHTLFYYGHINISSFFNLDKYFINLLNEKHKNILNYMSSFESVAIHIRRGDYMYALKIDGRKNNASLDYIYTSIKYLNNTLENKNIKFYIFSNNKKYIKNNLIPIFQNNNINFEIIEDNPEYIDFYLMTKCKHIISSLGKFAKVAFKFIKNENKIFIDTDNIYNLKKEYFYKNICEKYCNIIYEEYNNIKTYISKNNMIYILDLIEKNNIKNICQIGLLDGLETHTLLKYSADNNNCFNLLCFENNERDIIGIELKNLTENEKKLFTLYIEQSPLDIDNSFNIKNIDFIFFTKENSNPLVFIYLIYLFPFMKEEIIIVFNDIISGKYYSGSYCIFNMFNGEKNQFYNFNTNKYDNIGYIKIKKQELLNIIKKISVIEYDGYINKFFMNKLIDNRNDYYNYFDLDMSYIRLNNLKQYMKDKFDIEYSNYIISNIENNIINYEKQYYKNQKNNFYSQNQIHISKIENIIDKHNKLINSISWWIPIKKLRENFKNKFIRPDQTRPDQTRPDQTRPDLICKEYIYNILYYKINKLQYIN

SEQ ID NO: 89 - protein, 644 aa, Brachysprira hyodysenteriae
MNDKYVICSITTGFTDQLINFATAIYIREKYKKNVKLDISWFSVYENTDYLGIEKRNLEVLNIFNDFNFEIATKKEIDIALKSIFIDGRYENIDKILKSNKHIYVGPYVGHTLFYYGHINISSFFNLDKYFINLLNEKHKNILNDMSSFESVAIHIRRGDYMYALKIDGRKNNASLDYIYTSIKYLNNTLENKNIKFYIFSNNKKYIKNNLIPIFQNNNINFEIIEDNPEYIDFYLMTKCKHIISSLGKFAKVAFKFIKNENKIFIDTDNIYNLKKEYFYKNICEKYCNIIYEEYNNIKTYISKNNMIYILDLIEKNNIKNICQIGLLDGLETHTLLKYSADNNNCFNLLCFENNERDIVGIELKNLTENEKKLFTLYIEQSPLDIDNSFNIKNIDFIFFTKENSNPLVFIYLIYLFPFMKEEIIIVFNDIISGKYYSGSYCIFNMFNGEKNQFYNFNTNKYDNIGYIKIKKQELLNIIKKISVIEYDGYINKFFMNKLIDNRNDYYNYFDLDMSYIRLNNLKQYMKDKFDIEYSNYIISNIENNIINYEKQYYKNQKNNFYSQNQIHISKIENIIDKHNKLINSISWWIPIKKLRENFKNKFIRPDQTRPDQTRPDQTRPDLICKEYIYNILYYKINKLQYIN

SEQ ID NO: 90 - protein, 619 aa, Brachysprira hyodysenteriae
MNDKYVICSITTGFTDQLINFATAIYIREKYKKNVKLDISWFSVYENTDYLGIEKRNLEVLNIFNDFNFEIATKKEIDIALKSIFIDGRYENIDKILKSNKHIYVGPYVGHTLFYYGHINISSFFNLDKYFINLLNEKHKNILNDMSSFESVAIHIRRGDYMYALKIDGRKNNASLDYIYTSIKYLNNTLENKNIKFYIFSNNKKYIKNNLIPIFQNNNINFEIIEDNPEYIDFYLMTKCKHIISSLGKFAKVAFKFIKNENKIFIDTDNIYNLKKEYFYKNICEKYCNIIYEEYNNIKTYISKNNMIYILDLIEKNNIKNICQIGLLDGLETHTLLKYSADNNNCFNLLCFENNERDIVGIELKNLTENEKKLFTLYIEQSPLDIDNSFNIKNIDFIFFTKENSNPLVFIYLIYLFPFMKEEIIIVFNDIISDKYYSGSYCIFNMFNGEKNQFYNFNTNKYDNIGYIKIKKQELLNIIKKISVIEYDGYINKFFMNKLIDNRNDYYNYFDLDMSYIRLNNLKQYMKDKFDIEYSNYIIFNIENNIINYEKQYYKNQKNNFYSKNQIHISKIENIIDKHNKLINSISWWIPIKKLRENFKNKFIRPDQTRPDQTRPDQT

SEQ ID NO: 91 - protein, 619 aa, Brachysprira hyodysenteriae
MNDKYVICSITTGFTDQLINFATAIYIREKYKKNVKLDISWFSVYENTDYLGIEKRNLEVLNIFNDFNFEIATKKEIDIALKSIFIDGRYENIDKILKSNKHIYVGPYVGHTLFYYGHINISSFFNLDKYFINLLNEKHKNILNYMSSFESVAIHIRRGDYMYALKIDGRKNNASLDYIYTSIKYLNNTLENKNIKFYIFSNNKKYIKNNLIPIFQNNNINFEIIEDNPEYIDFYLMTKCKHIISSLGKFAKVAFKFIKNENKIFIDTDNIYNLKKEYFYKNICEKYCNIIYEEYNNIKTYISKNNMIYILDLIEKNNIKNICQIGLLDGLETHTLLKYSADNNNCFNLLCFENNERDIIGIELKNLTENEKKLFTLYIEQSPLDIDNSFNIKNIDFIFFTKENSNPLVFIYLIYLFPFMKEEIIIVFNDIISGKYYSGSYCIFNMFNGEKNQFYNFNTNKYDNIGYIKIKKQELLNIIKKISVIEYDGYINKFFMNKLIDNRNDYYNYFDLDMSYIRLNNLKQYMKDKFDIEYSNYIISNIENNIINYEKQYYKNQKNNFYSQNQIHISKIENIIDKHNKLINSISWWIPIKKLRENFKNKFIRPDQTRPDQTRPDQT

SEQ ID NO: 92 - protein, 623 aa, Brachysprira hyodysenteriae
MNDKYVICSITTGFTDQLINFATAIYIREKYKKNVKLDISWFSVYENTDYLGIEKRNLEVLNIFNDFNFEIATKKEIDIALKSIFIDGRYENIDKILKSNKHIYVGPYVGHTLFYYGHINISSFFNLDKYFINLLNKKHKNILNDMSSFESVAIHIRRGDYMYALKIDGRKNNASLDYIYTSIKYLNNTLENKNIKFYIFSNNKKYIKNNLIPIFQNNNINFEIIEDNPEYIDFYLMTKCKHIISSLGKFAKVAFKFIKNENKIFIDTDNIYNLKKEYFYKNICEKYCNIIYEEYNNIKTYISKNNMIYILDLIEKNNIKNICQIGLLDGLETHTLLKYSAYNNNCFNLLCFENNERDIVGIELKNLTENEKKLFTLYIEQSPLDIDNSFNIKNIDFIFFTKENSNPLVFIYLIYLFPFMKEEIIIVFNDIISGKYYSGSYCIFNMFNGEKNQFYNFNTNKYDNIGYIKIKKQELLNIIKKISVIEYDGYINKFFMNKLIDNRNDYYNYFDLDMSYIRLNNLKQYMKDKFDIEYSNYIISNIENNIINYEKQYYKNQKNNFYSQNQIHISKIENIIDKHNKLINSISWWIPIKKLRENFKNKFIRPDQTRPDQTRPDQTRPNM

SEQ ID NO: 93 - protein, 633 aa, Brachysprira hyodysenteriae
MNDKYVICSITTGFTDQLINFATAIYIREKYKKNVKLDISWFSVYENTDYLGIEKRNLEVLNIFNDFNFEIATKKEIDIALKSIFIDGRYENIDKILKSNKHIYVGPYVGHTLFYYGHINISSFFNLDKYFINLLNEKHKNILNDMSSFESVAIHIRRGDYMYALKIDGRKNNASLDYIYTSIKYLNNTLENKNIKFYIFSNNKKYIKNNLIPIFQNNNINFEIIEDNPEYIDFYLMTKCKHIISSLGKFAKVAFKFIKNENKIFIDTDNIYNLKKEYFYKNICEKYCNIIYEEYNNIKTYISKNNMIYILDLIEKNNIKNICQIGLLDGLETHTLLKYSADNNNCFNLLCFENNERDIVGIELKNLTENEKKLFTLYIEQSPLDIDNSFNIKNIDFIFFTKENSNPLVFIYLIYLFPFMKEEIIIVFNDIISDKYYSGSYCIFNMFNGEKNQFYNFNTNEYDNIGYIKIKKQELLNIIKKISVIEYDGYINKFFMNKLIDNRNDYYNYFDLDMSYIRLNNLKQYMKDKFDIEYSNYIISNIENNIINYEKQYYKNQKNNFYSKNQIHISKIENIIDKHNKLINSISWWIPIKKLRENFKNKFIRPDQTRPDQTRPDQTRPDQTRPDQTRPNM

SEQ ID NO: 94 - protein, 623 aa, Brachysprira hyodysenteriae
MNDKYVICSITTGFTDQLINFATAIYIREKYKKNVKLDISWFSVYENTDYLGIEKRNLEVLNIFNDFNFEIATKKEIDIALKSIFIDGRYENIDKILKSNKHIYVGPYVGHTLFYYGHINISSFFNLDKYFINLLNEKHKNILNYMSSFESVAIHIRRGDYMYALKIDGRKNNASLDYIYTSIKYLNNTLENKNIKFYIFSNNKKYIKNNLIPIFQNNNINFEIIEDNPEYIDFYLMTKCKHIISSLGKFAKVAFKFIKNENKIFIDTDNIYNLKKEYFYKNICEKYCNIIYEEYNNIKTYISKNNMIYILDLIEKNNIKNICQIGLLDGLETHTLLKYSADNNNCFNLLCFENNERDIVGIELKNLTENEKKLFTLYIEQSPLDIDNSFNIKNIDFIFFTKENSNPLVFIYLIYLFPFMKEEIIIVFNDIISGKYYSGSYCIFNMFNGEKNQFYNFNTNKYDNIGYIKIKKQELLNIIKKISVIEYDGYINKFFMNKLIDNRNDYYNYFDLDMSYIRLNNLKQYMKDKFDIEYSNYIISNIENNIINYEKQYYKNQKNNFYSQNQIHISKIENIIDKHNKLINSISWWIPIKELRENFKNKFIRPDQTRPDQTRPDQTRPNM

SEQ ID NO: 95 - protein, 618 aa, Brachysprira hyodysenteriae
MNDKYVICSITTGFTDQLINFATAIYIREKYKKNVKLDISWFSVYENTDYLGIEKRNLEVLNIFNDFNFEIATKKEIDIALKSIFIDGRYENIDKILKSNKHIYVGPYVGHTLFYYGHINISSFFNLDKYFINLLNEKHKNILNYMSSFESVAIHIRRGDYMYALKIDGRKNNASLDYIYTSIKYLNNTLENKNIKFYIFSNNKKYIKNNLIPIFQNNNINFEIIEDNPEYIDFYLMTKCKHIISSLGKFAKVAFKFIKNENKIFIDTDNIYNLKKEYFYKNICEKYCNIIYEEYNNIKTYISKNNMIYILDLIEKNNIKNICQIGLLDGLETHTLLKYSADNNNCFNLLCFENNERDIVGIELKNLTENEKKLFTLYIEQSPLDIDNSFNIKNIDFIFFTKENSNPLVFIYLIYLFPFMKEEIIIVFNDIISGKYYSGSYCIFNMFNGEKNQFYNFNTNKYDNIGYIKIKKQELLNIIKKISVIEYDGYINKFFMNKLIDNRNDYYNYFDLDMSYIRLNNLKQYMKDKFDIEYSNYIISNIENNIINYEKQYYKNQKNNFYSQNQIHISKIENIIDKHNKLINSISWWIPIKELRENFKNKFIRPDQTRPDQTRPNM

SEQ ID NO: 96 - protein, 395 aa, Brachysprira hyodysenteriae
MNKCECSQCKNREYDIPEYLVLEDDLKILDNERPLGISGHLRVKDEAMSLAECIDSCIDFLDELIITYNTSSDNTEEILKNYEKKYPEKIKLFHYKPNIIRYNKDEYNTKYSQIHYLDNYYNYGLSKIKYKYYVKIDADQIYFTDKLLKLRELLILDINQIKLSNNINKLLLINKIAWCIPIKKLRNKFRTYCIRKYFNKDKNYIYNGDENILSLDDFILLSRKRHSDKNISYDLAGFNLILHNNKLYLDTYWPMNGGRFDHVLYQASYSNKYFMNEIFEVNQTPHNRIILGMFWVHFGNIKHNFQYDDSFYLLEYNDIKNSKVENFSHKMKNIDNDDKIEKYIRDNTFIKYFDEDKKYLNNVFYNKYLTKPLDYAIKNNDELVNLYGKTIITVK

SEQ ID NO: 97 - protein, 395 aa, Brachysprira hyodysenteriae
MNKCECSQCQNREYDISEYLVLEDDLKILDNERPLGISGHLRVKDEAMSLAECIDSCIDFLDELIITYNTSSDNTEEILKNYEKKYPEKIKLFHYKPNIIRYNKDEYNTKYSQIHYLDNYYNYGLIKIKYKYYVKIDADQIYFTDKLLKLRELLILDINQIKLSNNINKLLLINKIAWCIPIKKLRNKFRTYCIRKYFNKDKNYIYNGDENILSLDDFILLSRKRHSDKNISYDLAGFNLILHNNKLYLDTYWPMNGGRFDHVLYQASYSNKYFMNEIFEVNQTPHNRIILGMFWVHFGNIKHNFQYDDSFYLLEYNDIKNSKVENFSHKMKNIDNDDKIEKYIRDNTFIKYFDEDKKYLNNVFYNKYLTKPLDYAIKNNDELVNLYGKTIITVK

SEQ ID NO: 98 - protein, 395 aa, Brachysprira hyodysenteriae
MNTCECSQCKNREYDISEYLVLEDDLKILDNERPLGISGHLRVKDEAMSLAECIDSCIDFLDELIITYNTSSDNTEEILKNYEKKYPEKIKLFHYKPNIIRYNKDEYNTKYSQIHYLDNYYNYGLIKIKYKYYVKIDADQIYFTDKLLKLRELLILDINQIKLSNNINKLLLINKIAWCIPIKKLRNKFRTYCIRKYFNKDKNYIYNGDENILSLDDFILLSRKRHSDKNISYDLAGFNLILHNNKLYLDTYWPMNGGRFDHVLYQASYSNKYFMNEIFEVNQTPHNRIILGMFWVHFGNIKHNFQYDDSFYLLEYNDIKNSKVENFSHKMKNIDNDDKIEKYIRDNTFIKYFDEDKKYLNNVFYNKYLTKPLDYAIKNNDELVNLYGKTIITVK

SEQ ID NO: 99 - protein, 395 aa, Brachysprira hyodysenteriae
MNKCECSQCKNREYDISEYLVLEDDLKILDNERPLGISGHLRVKDEAMSLAECIDSCIDFLDELIITYNTSSDNTEEILKNYEKKYPEKIKLFHYKPNIIRYNKDEYNTKYSQIHYLDNYYNYGLIKIKYKYYVKIDADQIYFTDKLLKLRELLILDINQIKLSNNINKLLLINKIAWCIPIKKLRNKFRTYCIRKYFNKDKNYIYNGDENILSLDDFILLSRKRHSDKNISYDLAGFNLILHNNKLYLDIYWPMNGGRFDHVLYQASYSNKYFMNEIFEVNQTPHNRIILGMFWVHFGNIKHNFQYDDSFYLLEYNDIKNSKVENFSHKMKNIDNDDKIEKYIRDNTFIKYFDEDKKYLNNVFYNKYLTKPLDYAIKNNDELVNLYGKTIITVK

SEQ ID NO: 100 - protein, 287 aa, Brachysprira hyodysenteriae
MKGIVLAGGSGTRLYPMTNVISKQLLPIYDKPMVYYPLSVLMLSKIKDILVISTENDTPLFKKLLKDGRQWGCNIEYAVQKAPNGLAEAFIIGESFIGKDNVSMILGDNIFFGQSFSSVLEKSAKIDNGAVIFAYQVKDPERFGIVEIDKDYNALSIEEKPKNPKSNYAVTGLYFYDNDVVNISKNIKPSARGELEITDVNKVYLENKKLKVEVLGRGFAWLDTGTRDSLLQASQFVSAVENIEGMQIACLEEIAYNNKWIDASKIEETASKLKNSEYGKYLINLIK

SEQ ID NO: 101 - protein, 287 aa, Brachysprira hyodysenteriae
MKGIVLAGGSGTRLYPMTNVISKQLLPIYDKPMVYYPLSVLMLSKIKDILVISTENDTPLFKKLLKDGSQWGCNIEYAVQKAPNGLAEAFIIGESFIGKDNVSMILGDNIFFGQSFSSVLEKSAKIYNGAVIFAYQVKDPERFGIVEIDKDYNALSIEEKPKNPKSNYAVTGLYFYDNDVVNISKNIKPSARGELEITDVNKVYLENKKLKVEVLGRGFAWLDTGTRDSLLQASQFVSAVENIEGMQIACLEEIAYNNKWIDASKIEETASKLKNSEYGKYLINLIK

SEQ ID NO: 102 - protein, 287 aa, Brachysprira hyodysenteriae
MKGIVLAGGSGTRLYPMTNVISKQLLPIYDKPMVYYPLSVLMLSKIKDILVISTENDTPLFKKLLKDGSQWGCNIEYAVQKAPNGLAEAFIIGESFIGKDNVSMTLGDNIFFGQSFSSVLEKSAKIDNGAVIFAYQVKDPERFGIVEIDKDYNALSIEEKPKNPKSNYAVTGLYFYDNDVVNISKNIKPSARGELEITDVNKVYLENKKLKVEVLGRGFAWLDTGTRDSLLQASQFVSAVENIEGMQIACLEEIAYNNKWIDASKIEETASKLKNSEYGKYLINLIK

SEQ ID NO: 103 - protein, 287 aa, Brachysprira hyodysenteriae
MKGIVLAGGSGTRLYPMTNVISKQLLPIYDKPMVYYPLSVLMLSKIKDILVISTENDTPLFKKLLKDGSQWGCNIEYAVQKAPNGLAEAFIIGESFIGKDNVSMILGDNIFFGQSFSSVLEKSAKIDNGAVIFAYQVKDPERFGIVEIDKDYNALSIEEKPKNPKSNYAVTGLYFYDNDVVNISKNIKPSARGELEITDVNKVYLENKKLKVEVLGRGFAWLDTGTRDSLLQASQFVSAVENIEGMQIACLEEIAYNNKWIDASIIEETASKLKNSEYGKYLINLIK

SEQ ID NO: 104 - protein, 287 aa, Brachysprira hyodysenteriae
MKGIVLAGGSGTRLYPMTNVISKQLLPIYDKPMVYYPLSVLMLSKIKDILVISTENDTPLFKKLLKDGSQWGCNIEYAVQKAPNGLAEAFIIGESFIGKDNVSMILGDNIFFGQSFSSVLEKSAKIDNGAVIFAYQVKDPERFGIVEIDKDYNALSIEEKPKNPKSNYAVTGLYFYDNDVVNISKNIKPSARGELEITDVNKVYLENKKLKVEVLGRGFAWLDTGTRDSLLQASQFVSAVENIEGMQIACLEEIAYNNKWIDASKIEETASKLKNSEYGKYLINLIK

SEQ ID NO: 105 - protein, 356 aa, Brachysprira hyodysenteriae
MRNFNNILVTGGCGFIGTNFIRYILQKTEYKGNIINIDALTYAGNRENLIDIEKKYKDRYFFEKVNICDDKKVNEIFSQYNPDCIVHFAAESHVDRSIYGPKDFLETNIMGTFILLEAARKLWENNKENKLFHHISTDEVYGSLGETGYFYETTPYDPRSPYSSSKASSDHIVKAYYHTYNLPVTISNCSNNYGPYQFPEKLIPLMISNIVEEKDLPVYGDGKNIRDWIFVEDHNNAVIDIIHKGRVGETYNIGGENEITNIDMVNILCEKLSAKMNKEKDYYKKLIKFVKDRSGHDRRYAINCDKIKKELGWERKYDFNTAIDITIDWYLNNKQWIDNIKSGEYKKWIELNYSSK

SEQ ID NO: 106 - protein, 356 aa, Brachysprira hyodysenteriae
MRNFNNILVTGGCGFIGSNFIRYILEKSDYKGNIINIDALTYAGNRENLIDIEKKYKDRYFFEKVNICDDKKINEIFSKYNPDCIVHFAAESHVDRSIYGPKDFLETNIMGTFILLEAARKLWENNKENKLFHHISTDEVYGSLGETGYFYETTPYDPRSPYSSSKASSDHIVKAYYHTYNLPVTISNCSNNYGPYQFPEKLIPLMISNIVEEKDLPVYGDGKNIRDWIFVEDHNNAVIDIIHKGRVGETYNIGGENEITNIDMVNILCEKLSAKMNKEKDYYKKLIKFVKDRSGHDRRYAINCDKIKKELGWERKYDFNTAIDITIDWYLNNKQWIDNIKSGEYKKWIELNYSSK

SEQ ID NO: 107 - protein, 356 aa, Brachysprira hyodysenteriae
MRNFNNILVTGGCGFIGSNFIRYILEKSDYKGNIINIDALTYAGNRENLIDIEKKYKDRYFFEKVNICDDKKINEIFSKYNPDCIVHFAAESHVDRSIYGPKDFLETNIMGTFILLEAARKLWENNKENKLFHHISTDEVYGSLGETGYFYETTPYDPRSPYSSSKASSDHIVKAYYHTYNLPVTISNCSNNYGPYQFPEKLIPLMISNIVEEKDLPVYGDGKNIRDWIFVEDHNNAVIDIIHKGRVGETYNIGGENEITNIDMVNILCEKLSAKMNKEKDYYKKLIKFVKDRAGHDRRYAINCDKIKKELGWERKYDFNTGIDITIDWYLNNKQWIDNIKSGEYKKWIELNYSSK

SEQ ID NO: 108 - protein, 356 aa, Brachysprira hyodysenteriae
MRNFNNILVTGGCGFIGSNFIRYILEKSDYKGNIINIDALTYAGNRENLIYIEKKYKDRYFFEKVNICDDKKINEIFSKYNPDCIVHFAAESHVDRSIYGPKDFLETNIMGTFILLEAARKLWENNKENKLFHHISTDEVYGSLGETGYFYETTPYDPRSPYSSSKASSDHIVKAYYHTYNLPVTISNCSNNYGPYQFPEKLIPLMISNIVEEKDLPVYGDGKNIRDWIFVEDHNNAVIDIIHKGRVGETYNIGGENEITNIDMVNILCEKLSAKMNKEKDYYKKLIKFVKDRAGHDRRYAINCDKIKKELGWERKYDFNTAIDITIDWYLNNKQWIDNIKSGEYKKWIELNYSSK

SEQ ID NO: 109 - protein, 192 aa, Brachysprira hyodysenteriae
MPFTFKKLDINGLFIAEPRIFGDSRGYFLETYSEKDFFEAGIKEKFVQDNQSKSTKNVLRGLHYQKKYCQAKLVRVLEGEVFDVAVDLRNDSPTFGKYYGVLLTEENKKQFFIPKGFAHGFIVLSESAVFTYKCSDFYHPEEEGGIIYNDKNINIDWTCDTKDVLVSDKDLKLPEFDKNKKYFSLDGNWIGE

SEQ ID NO: 110 - protein, 192 aa, Brachysprira hyodysenteriae
MPFTFKKLDINGLFIAEPRIFGDSRGYFLETYSEKDFFEAGIKEKFVQDNQSKSTKNVLRGLHYQKKYCQAKLVRVLEGKVFDVAVDLRNDSPTFGKYYGVLLTEENKKQFFIPKGFAHGFIVLSESAVFAYKCSDFYHPEEEGGIIYNDKNINIDWTCDTKDVLVSDKDLKLPEFDKNKKYFSLDGNWIGE

SEQ ID NO: 111 - protein, 192 aa, Brachysprira hyodysenteriae
MPFTFKKLDINGLFIAEPRIFGDSRGYFLETYSEKDFFEAGIKEKFVQDNQSKSTKNVLRGLHYQKKYCQAKLVRVLEGEVFDVAVDLRNDSPTFGKYYGVLLTEENKKQFFIPKGFAHGFIVLSESAVFAYKCSDFYHPEEEGGIIYNDKNINIDWTCDTKDVLVSDKDLKLLEFDKNKKYFSLDGNWIGE

SEQ ID NO: 112 - protein, 293 aa, Brachysprira hyodysenteriae
MIWVIGKNGMLAQDILKAFNKNNIEYTSTASNIDITNIDILNNFIKDKNIKTIINCSAYTKVDLAEDEKDICYKVNGEGVKNITEIASNINADLIHFSTDYVFDGENNKPYNEEDKTNPINIYGKSKLEGEKYALFYNKAIVIRVSWLYGIYGKNFVYTMINLMNSKESIKVVNDQFGSPTYTEDVAMVIYDIIKNNNFDYGLYHYTNEGNISWYDFAKTIYNKGKEYNIINNDCKVNPCTTEEYPTKAKRPKYSVLSVEKIKRYVKIYNYEDSLNSFLTYIKKINNFWSLLV

SEQ ID NO: 113 - protein, 286 aa, Brachysprira hyodysenteriae
MIWIIGKNGMLAQDILQVFNKNNIEYTSTASNIDITNIDILNNFIKDKNIKTIINCSAYTKVDLAEDEKDICYKVNGEGVKNITEIASNINADLIHFSTDYVFDGENNKPYNEEDKTNPINIYGKSKLEGEKYALSYNKSIVIRVSWLYGIYGKNFVYTMINLMNSKESIKVVNDQFGSPTYTEDVAMVIYDIIKNNNFYYGLYHYTNEGDISWYDFAKTIYNKGKEYNIINNDCKVNPCTTEEYPTKAKRPKYSVLSVEKIKRYVKIYNYEDSLNSFFNIYQKNK

SEQ ID NO: 114 - protein, 286 aa, Brachysprira hyodysenteriae
MIWIIGKNGMLAQDILQVFNKNNIEYTSTASNIDITNIDILNNFIKDKNIKTIINCSAYTKVDLAEDEKDICYKVNGEGVKNITEIASNINADLIHFSTDYVFDGENNKPYNEEDKTNPINIYGKSKLEGEKYALSYNKSIVIRVSWLYGIYGKNFVYTMINLMNSKESIKVVNDQFGSPTYTEDVAMVIYDIIKNNNFDYGLYHYTNEGDISWYDFAKTIYNKGKEYNIINNDCKVNPCTTEEYPTKAKRPKYSVLSVEKIKRYVKIYNYEDSLNSFFNIYQKNK

SEQ ID NO: 115 - protein, 286 aa, Brachysprira hyodysenteriae
MIWIIGKNGMLAQDILQVFNKNNIEYTSTASNIDITNIDILNNFIKDKNIKTIINCSAYTKVDLAEDEKDICYKVNGEGVKNITEIASNINADLIHFSTDYVFDGENNKPYNEEDKTNPINIYGKSKLEGEKYALFYNKAIVIRVSWLYGIYGKNFVYTMINLMNSKESIKVVNDQFGSPTYTEDVAMVIYDIIKNNNFDYGLYHYTNEGNISWYDFAKTIYNKGKEYNIINNDCKVNPCTTEEYPTKAKRPKYSVLSVEKIKRYVKIYNYEDSLNSFFNIYQKNK

SEQ ID NO: 116 - protein, 286 aa, Brachysprira hyodysenteriae
MIWVIGKNGMLAQDILKAFNKNNIEYTSTASNIDITNVDILNNFIKDKNIKTIINCSAYTKVDLAEDEKDICYKVNGEGVKNITEIASNINADLIHFSTDYVFDGENNKPYNEEDKTNPINIYGKSKLEGEKYALFYNKAIVIRVSWLYGIYGKNFVYTMINLMNSKESIKVVNDQFGSPTYTEDVAMVIYDIIKNNNFDYGLYHYTNEGNISWYDFAKTIYNKGKEYNIINNDCKVNPCTTEEYPTKAKRPKYSVLSVEKIKRYVKIYNYEDSLNSFFNIYQKNK

SEQ ID NO: 117 - protein, 286 aa, Brachysprira hyodysenteriae
MIWVIGKNGMLAQDILKAFNKNNIEYTSTASNIDITNIDILNNFIKDQNIKTIINCSAYTKVDLAEDEKDICYKVNGEGVKNITEIASNINADLIHFSTDYVFDGENNKPYNEEDKTNPINIYGKSKLEGEKYALFYNKAIVIRVSWLYGIYGKNFVYTMINLMNSKESIKVVNDQFGSPTYTEDVAMVIYDIIKNNNFDYGLYHYTNEGNISWYDFAKTIYNKGKEYNIINNDCKVNPCTTEEYPTKAKRPKYSVLSVEKIKRYVKIYNYEDSLNSFFNIYQKNK

SEQ ID NO: 118 - protein, 326 aa, Brachysprira hyodysenteriae
MKRLAVFAGYDKDNIIDDYVVYYIKELKKIADIIYVSDCNMSDNELKKINNYCIHIINGRHEEYDFGSYKRGYLYAKENNILQNYDYLILCNDSCYGPLFDLENIVNKMNDYDFGGISISKDLKIANHNYITSFFIYINKNIFNKDFFNDFIYDIKKEEDKMDIIKKYEFGLSKLMLDNNIELKGLFNDNGEFNRPYFNPLALIEEGYPFLKKHVLEKKVTVPLNIDELTNIIKIIKNNYDIKLIVNHLNRVADKEQIKYLFQKYKPYKKTFIHEKIFSLFTRYSPSGKYQTVYKFFNFLTISIDKPIKDSYIETDYKDFNFLLKI

SEQ ID NO: 119 - protein, 326 aa, Brachysprira hyodysenteriae
MKRLAVFAGYDKDNIIDDYVVYYIKELKKIADIIYVSDCNMSDNELKKINNYCIHIINGRHEEYDFGSYKRGYLYAKENNILQNYDYLILCNDSCYGPLFDLENIVNKMNDYDFGGISISKDLKIANHNYITSFFIYINKNIFNKDFFNDFIYDIKKEEDKMDIIKKYEFGLSKLMLDNNIELKGLFNDNGEFNRPYFNPLALIEEGYPFLKKHVLEKKVTVPLNIDELTNIIKIIKNNYDIKLIVNHLNRVADKEQIKYLFQKYKPYKKTFIHEKIFSLFTRYSPSGKYQTVYNFFNFLTISIDKPIKDSYIETDYKDFNFLLEI

SEQ ID NO: 120 - protein, 326 aa, Brachysprira hyodysenteriae
MKRLAVFAGYDKDNIIDDYVVYYIKELKKIADIIYVSDCNMSDNELKKINDYCIHIINGRHEEYDFGSYKRGYLYAKENNILQNYDYLILCNDSCYGPLFDLENIINKMNNYDFGGISISKDLKIANHNYITSFFIYINKNIFNKDFFNDFIFNIKKEEDKMDIIKKYEFGLSKLMLDNNIELKGLFNDNGEFNRPYFNPLALIEEGYPFLKKHVLEKKITVPLNINELTNIIKIIKNNYDIKLIVNHLNRVADKEQIKYLFQKYKPYKKIFIHEKIFSLFTRYSPSGKYQTVYKFFNFLTISIDKPIKDSYIETDYKDFNFLLEI

SEQ ID NO: 121 - protein, 326 aa, Brachysprira hyodysenteriae
MKRLAVFAGYDKDNIIDDYVVYYIKELKKISDIIYVSDCNMSDNELKKINDYCIHIINGRHEEYDFGSYKRGYLYAKENNILQNYDYLILCNDSCYGPLFDLENIINKMNDYDFGGISISKDLKIANHNYITSFFIYINKNIFNKDFFNDFIYDIKKEEDKMDIIKKYEFGLSKLMLDNNIELKGLFNDNGEFNRPYFNPLALIEEGYPFLKKHVLEKKVTVPLNIDELTNIIKIIKNNYDIKLIVNHLNRVADKEQIKYLFQKYKPYKKTFIHEKIFSLFTRYSPSGKYQTVYKFFNFLTISIDKPIKDSYIETDYKDFNFLLEI

SEQ ID NO: 122 - protein, 326 aa, Brachysprira hyodysenteriae
MKRLAVFAGYDKDNIIDDYVVYYIKELKKISDIIYVSDCNMSDNELKKINDYCIHIINGRHEEYDFGSYKRGYLYAKENNILQNYDYLILCNDSCYGPLFDLENIINKMNDYDFGGISISKDLKIANHNYITSFFIYINKNIFNKDFFNDFIYDIKKEEDKMDIIKKYEFGLSKLMLDNNIELKGLFNDNGEFNRPYFNPLALIEEGYPFLKKHVLEKKVTVPLNIDELTNIIKIIKNNYDIKLIVNHLNRVADKEQIKYLFQKYKPYKKTFIHEKIFSLFTRYSPSGKYQTVYNFFNFLTISIDKPIKDSYIETDYKDFNFLLEI

SEQ ID NO: 123 - protein, 326 aa, Brachysprira hyodysenteriae
MKRLAVFAGYDKDNIIDDYVVYYIKELKKISDIIYVSDCNMSDNELKKINDYCIHIINGRHEEYDFGSYKRGYLYAKENNILQNYDYLILCNDSCYGPLFDLENIINKMNDYDFGGISISKDLKIANHNYITSFFIYINKNIFNKDFFNNFIYDIKKEEDKMDIIKKYEFGLSKLMLDNNIELKGLFNDNGEFNRPYFNPLALIEEGYPFLKKHVLEKKVTVPLNIDELTNIIKIIKNNYDIKLIVNHLNRVADKEQIKYLFQKYKPYKKTFIHEKIFSLFTRYSPSGKYQTVYNFFNFLTISIDKPIKDSYIETDYKDFNFLLEI

(19) United States
(12) Patent Application Publication
(10) Pub. No.: US 2024/0108708 A1
(43) Pub. Date: Apr. 4, 2024

(54) IMMUNOGENIC AND VACCINE COMPOSITIONS AGAINST SWINE DYSENTERY

(71) Applicant: HIPRA SCIENTIFIC, S.L.U., Amer, Girona (ES)

(72) Inventors: Jesús María OSORIO ARGUELLO, Amer, Girona (ES); Jaume PIÑOL RIBAS, Valldoreix, Barcelona (ES); Luis GONZÁLEZ GONZÁLEZ, Amer, Girona (ES); Jordi MONTANÉ GIRALT, Amer, Girona (ES); Marta SITJÀ ARNAU, Amer, Girona (ES); Enrique QUEROL MURILLO, Barcelona (ES)

(21) Appl. No.: 18/259,633

(22) PCT Filed: Dec. 28, 2021
(86) PCT No.: PCT/EP2021/087718

§ 371 Date: Jun. 28, 2023

(30) Foreign Application Priority Data
Dec. 29, 2020 (EP) 20383166.4

(51) Int. Cl. A61K 39/02; A61P 31/04

(52) U.S. Cl. CPC A61K 39/0225; A61P 31/04; A61K 2039/521

(57) ABSTRACT

The invention relates to an immunogenic or vaccine composition comprising an inactivated bacterium from the species Brachyspira hyodysenteriae, to a method for producing said composition and to the medical use of this composition for inducing an immune response against a bacterium from the species Brachyspira hyodysenteriae or for protecting against an infection caused by a bacterium from the species Brachyspira hyodysenteriae. Further, the invention relates to a method for selecting a bacterium from the species Brachyspira hyodysenteriae useful for manufacturing a vaccine against swine dysentery.

### FIELD OF THE INVENTION

The present invention belongs to the field of diseases caused by intestinal spirochaetes. Particularly, the invention relates to novel immunogenic and vaccine compositions for swine dysentery. More particularly, the invention relates to novel immunogenic and vaccine compositions for the prevention and/or treatment of infections caused by Brachyspira hyodisenteriae.

### BACKGROUND OF THE INVENTION

Brachyspira spp. are anaerobic intestinal spirochaetes that can be isolated from pigs, and other mammals, including birds and humans. In pigs, seven Brachyspira spp. have been described, i.e. Brachyspira hyodysenteriae, Brachyspira pilosicoli, Brachyspira intermedia, Brachyspira murdochii, Brachyspira innocens, Brachyspira suanatina and Brachyspira hampsonii. B. hyodysenteriae is especially relevant in pigs as it causes swine dysentery and hence considerable economic losses to the pig industry. Furthermore, reduced susceptibility of B. hyodysenteriae to antimicrobials is of increasing concern. The epidemiology of B. hyodysenteriae infections is only partially understood, but different methods for detection, identification and typing have supported recent improvements in knowledge and understanding.

B. hyodysenteriae (previously known as Serpulina or Treponema hyodysenteriae) is the etiologic agent of swine dysentery (SD), a severe, infectious disease characterized by mucohemorrhagic diarrhea and marked inflammation limited to the large intestine (cecum and/or colon). B. hyodysenteriae is the specie with the highest incidence in pig farms with a high economic impact on affected farms worldwide.

Different approaches have been used to control SD, varying from the prophylactic use of antimicrobial agents, to complete destocking of infected herds and prevention of re-entry of infected carrier pigs. All these options are expensive and, if they are to be fully effective, they require the use of tedious diagnostic tests to monitor progress.

Since an immunological response is induced following colonization with B. hyodysenteriae, pigs recovered from SD are protected from re-infection. Despite of this fact, attempts to develop vaccines to control the disease had very limited success, either because they have provided inadequate protection on a herd basis, or they have been too costly and difficult to produce on a large scale for a feasible commercial use (WO2017/118581).

Vaccines based on inactivated B. hyodysenteriae isolates were reported to have limited success, but the main disadvantage of the inactivated vaccines is that they do not provide protection against different strains/serotypes (Olson et al., American Journal of Veterinary Research, 1994, 67-71; Diego et al., Vaccine, 1995, 663-667).

Autogenous preparations, also known as “autogenous vaccines” or “autovaccines” are vaccines prepared from the cultures of the microorganisms isolated from the animal's own tissues or secretions in presence of the infection. These autogenous preparations have also been used to develop vaccines against SD. However, this approach is costly and time consuming and it has shown that confers protection exclusively against the same strain of B. hyodysenteriae that causes clinical signs of the infected pigs.

Other examples of inactivated based B. hyodysenteriae vaccines are: (i) those disclosed in the patent family belonging to the PCT Publication No. WO2014/207202 filed by Aquilon CyL and Universidad de León. The Applicants disclose a universal vaccine against swine dysentery comprising specific bacteria from at least two genetically diverse strains of B. hyodysenteriae, and (ii) those disclosed in the patent family belonging to the PCT Publication No. WO2019/081583 filed by Aquilon CyL, where it is disclosed a single strain dysentery vaccine comprising a particular B. hyodysenteriae bacteria. Despite these attempts to develop vaccines to control SD, they all had very limited success, because they have provided inadequate protection on a herd basis, particularly to provide cross-protection against heterologous B. hyodysenteriae strains, and they have been too costly and difficult to produce to make them commercially viable.

Regarding the use of attenuated vaccines different approaches have also been disclosed, mostly based on B. hyodysenteriae mutants which are defective in one or more virulence factors or hemolysins associated genes.

An example of attenuated vaccine is described in the patent document WO2017/118581, filed by the Universiteit Gent. This document discloses a strain of B. hyodysenteriae that is useful as live attenuated vaccine.

Recombinant subunit vaccines have also been described. However, none of them have achieved significant protection.

Despite such efforts, there is no commercial vaccine available to protect swine against different strains of B. hyodysenteriae.

Therefore, there remains a need for novel, safe and effective vaccines against B. hyodysenteriae infections in swine, and particularly vaccines that confer heterologous protection against different B. hyodysenteriae strains.

### SUMMARY OF THE INVENTION

The authors of the present invention have found that B. hyodysenteriae strains can be classified into four different groups (S0, S1, S2 and S3) based on the structure of a gene cluster located on a plasmid of about 36 kb (pBH) and comprising, among others, genes from the rfbBADC operon. The inventors have also shown that vaccines comprising inactivated B. hyodysenteriae strains pertaining to the S1 and/or S2 group are effective in treating and/or preventing infections caused by B. hyodysenteriae, even if the infection was caused by a heterologous strain. Additionally, multivalent vaccines comprising strains pertaining to the S1 and S2 groups have higher efficacy than autogenous vaccines, and are also able to confer cross-protection against heterologous B. hyodysenteriae strains.

Therefore, the authors of the present invention have surprisingly found a novel gene cluster located on the plasmid pBH which is associated with obtaining effective protection against SD, particularly for conferring cross-protection between heterologous B. hyodysenteriae strains.

Therefore, in a first aspect, the invention relates to an immunogenic or vaccine composition comprising an isolated inactivated bacterium from the species Brachyspira hyodysenteriae, wherein said bacterium comprises the gene on the locus BHWA1_02689 (fucT) of the B. hyodysenteriae plasmid pBHWA1 identified by the GenBank accession number NC_012226.1 or a variant of said gene of any strain of B. hyodysenteriae.

In a second aspect, the invention relates to a method for producing an immunogenic or vaccine composition comprising an isolated bacterium from the species Brachyspira hyodysenteriae, wherein the method comprises the steps of:
(a) determining if the bacterium from the species Brachyspira hyodysenteriae comprises the gene on the locus BHWA1_02689 (fucT) of the B. hyodysenteriae plasmid pBHWA1 identified by the GenBank accession number NC_012226.1 or a variant of said gene of any strain of B. hyodysenteriae, or the product of said gene or variant,
(b) selecting the bacterium from the species Brachyspira hyodysenteriae if said bacterium comprises the fucT gene or a variant of said gene of any strain of B. hyodysenteriae or the product of said gene or variant,
(c) inactivating the bacterium selected in step (b) and
(d) formulating the bacterium inactivated in step (c) in an immunogenic or vaccine composition.

In another aspect, the invention relates to an immunogenic or vaccine composition obtainable by the method of the second aspect.

In another aspect, the invention relates to a method for selecting a bacterium from the species Brachyspira hyodysenteriae useful for manufacturing a vaccine against swine dysentery comprising determining the presence or absence of the gene on the locus BHWA1_02689 (fucT) of the B. hyodysenteriae plasmid pBHWA1 identified by the GenBank accession number NC_012226.1 or a variant of said gene of any strain of B. hyodysenteriae and/or of the product of said gene or variant, wherein if the bacterium comprises said gene or variant or the product of said gene or variant, the bacterium is selected for manufacturing a vaccine against swine dysentery.

In another aspect, the invention relates to the immunogenic or vaccine composition of the first aspect for use as a medicament.

In another aspect, the invention relates to the immunogenic composition of the first aspect for use in inducing an immune response against a bacterium from the species Brachyspira hyodysenteriae.

In another aspect, the invention relates to the vaccine composition of the first aspect for use in the protection against an infection caused by a bacterium from the species Brachyspira hyodysenteriae.

### BRIEF DESCRIPTION OF THE DRAWINGS

FIG. 1. Genomic structures of the gene cluster comprising the rfbBADC operon and the different genetic structures found (S1 to S3). The different genes are identified.

FIG. 2. Mean diarrhea score of pigs from day 1 to 20 after the experimental infection, groups A to E. Group A corresponds to pigs vaccinated with inactivated strain B-7493 (gene cluster structure S1); Group B corresponds to pigs vaccinated with inactivated strain B-7500 (gene cluster structure S1); Group C corresponds to pigs vaccinated with inactivated strain B-7843 (gene cluster structure S3); Group D corresponds to pigs vaccinated with a mock vaccine; Group E corresponds to negative control group, mock vaccinated-no challenged group. The diarrhea score is represented on the y-axis.

FIG. 3. Mean intestinal lesion score of pigs after the experimental infection, groups A to E. Group A corresponds to pigs vaccinated with inactivated strain B-7493 (gene cluster structure S1); Group B corresponds to pigs vaccinated with inactivated strain B-7500 (gene cluster structure S1); Group C corresponds to pigs vaccinated with inactivated strain B-7843 (gene cluster structure S3); Group D corresponds to pigs vaccinated with a mock vaccine; Group E corresponds to negative control group, mock vaccinated—no challenged group. The intestinal lesion score is represented on the y-axis.

FIG. 4: Mean diarrhea score of pigs from day 1 to 10 after the experimental infection, groups A to F. Group A corresponds to pigs vaccinated with inactivated strain B-7500 (gene cluster structure S1); Group B corresponds to pigs vaccinated with inactivated strain B-7484 (gene cluster structure S2); Group C corresponds to pigs vaccinated with inactivated strain B-7494 (gene cluster structure S2); Group D corresponds to pigs vaccinated with inactivated strain B-7843 (gene cluster structure S3); Group E corresponds to pigs vaccinated with a mock vaccine; Group F corresponds to negative control group, mock-vaccinated-no infected group. The diarrhea score is represented on the y-axis.

FIG. 5. Survival percentage of pigs from day 1 to 20 after the experimental infection (Days 36 to 56 of the study), groups A to E. Group A corresponds to pigs vaccinated with inactivated strains B-7500 and B-8295 (both with gene cluster structure S1); Group B corresponds to pigs vaccinated with inactivated strains B-7500, B-8295, and B-8397 (gene cluster structure S2); Group C corresponds to pigs vaccinated with inactivated strains B-7500, B-8295, B-8397, and B-7484 (gene cluster structure S2); Group D corresponds to pigs vaccinated with inactivated strains B-7500, B-8295, B-8397, B-7484, and B-7482 (gene cluster structure S1); Group E corresponds to pigs vaccinated with a mock vaccine. The percentage of pig survival is represented on the y-axis. Days after experimental infection are represented on the x-axis.

FIG. 6. Mean intestinal lesion score of pigs, euthanized after receiving the experimental infection at day 58, groups A to E. Group A corresponds to pigs vaccinated with inactivated strains B-7500 and B-8295 (both with gene cluster structure S1); Group B corresponds to pigs vaccinated with inactivated strains B-7500, B-8295, and B-8397 (gene cluster structure S2); Group C corresponds to pigs vaccinated with inactivated strains B-7500, B-8295, B-8397, and B-7484 (gene cluster structure S2); Group D corresponds to pigs vaccinated with inactivated strains B-7500, B-8295, B-8397, B-7484, and B-7482 (gene cluster structure S1); Group E corresponds to pigs vaccinated with a mock vaccine. The intestinal lesion score is represented on the y-axis.

FIG. 7. Survival percentage of pigs from day 1 to 20 after the experimental infection (Days 36 to 56 of the study), groups A to F. Groups A, C and E correspond to pigs vaccinated with a vaccine comprising inactivated strains B-7500 (gene cluster structure S1), B-8295 (gene cluster structure S1), and B-8397 (gene cluster structure S2); Groups B, D and F correspond to pigs vaccinated with a mock vaccine. Groups A and B were experimentally infected with the strain B-7493; Groups C and D were experimentally infected with the strain B-7482; Groups E and F were experimentally infected with the strain B-7484. The percentage of pig survival is represented on the y-axis. Days after experimental infection are represented on the x-axis.

FIG. 8. Mean intestinal lesion score of pigs, euthanized after receiving the experimental infection at day 57, groups A to F. Groups A, C and E correspond to pigs vaccinated with a vaccine comprising inactivated strains B-7500 (gene cluster structure S1), B-8295 (gene cluster structure S1), and B-8397 (gene cluster structure S2); Groups B, D and F correspond to pigs vaccinated with a mock vaccine. Groups A and B were experimentally infected with the strain B-7493; Groups C and D were experimentally infected with the strain B-7482; Groups E and F were experimentally infected with the strain B-7484. The intestinal lesion score is represented on the y-axis.

### DETAILED DESCRIPTION OF THE INVENTION

### Immunogenic or Vaccine Composition

In a first aspect, the invention relates to an immunogenic or vaccine composition comprising an isolated inactivated bacterium from the species Brachyspira hyodysenteriae, wherein said bacterium comprises the gene on the locus BHWA1_02689 (fucT gene) of the B. hyodysenteriae plasmid pBHWA1 identified by the GenBank accession number NC_012226.1 or a variant of said gene of any strain of B. hyodysenteriae.

Throughout the present description and in the claims, the expressions in singular preceded by the articles “a” or “the” are understood to also include, in a broad manner, the reference to the plural, unless the context clearly indicates the contrary.

The term “immunogenic composition”, as used herein, refers to a composition that elicits an immune response in a subject that has been exposed to said composition. An immune response may induce antibodies and/or cell-mediated immune responses against a specific immunogen. Thus, an immune response in particular means but is not limited to the development in a subset of a cellular and/or antibody-mediated immune response to the composition or vaccine of the invention. Immunogenic compositions can be prepared, for instance, as injectable pharmaceutic form such as liquid solutions, suspensions, emulsions, and freeze-dried compositions. The immunogenic composition comprises components with antigenic properties, such as immunogenic polypeptides, lipoligosaccharides, glycoconjugates, whole killed bacterium, supernatants of microorganism cultures, including bacteria, protozoa and viruses. A molecule is “antigenic” when it is capable of specifically interacting with an antigen recognition molecule of the immune system, such as an immunoglobulin (antibody) or T cell antigen receptor.

The term “antigen” refers to a component against which a subject can initiate an immune response, e.g. humoral and/or cellular immune response. Depending on the intended function of the composition, one or more antigens may be included.

The term “vaccine composition”, as used herein, refers to a composition that establishes or improves immunity to a particular disease by inducing an adaptive immune response including an immunological memory. A vaccine typically contains an agent that resembles a disease-causing microorganism or a part thereof (e.g. a polypeptide). Vaccines can be prophylactic or therapeutic. The term “vaccine composition”, as also used herein, refers to an immunogenic composition of the invention complemented by pharmaceutically acceptable excipients and/or carriers, that when administered to a subject, elicits, or is able to elicit directly or indirectly, an immune response in the host or subject. Particularly, the vaccines of the present invention elicit an immunological response in the host or subject of a cellular or antibody-mediated type upon administration to the subject that it is protective. The vaccine may be a “combination vaccine”. The term “combination vaccine” means that the vaccine contains various antigens in a single preparation. The vaccine composition of the invention can be also administered in combination with other vaccine compositions.

The term “bacterium from the species Brachyspira hyodysenteriae”, as used herein, refers to a spirochete bacterium of a species identified in the NCBI database with the Taxonomy ID: 159. The species Brachyspira hyodysenteriae was previously named Serpulina hyodysenteriae and Treponema hyodysenteriae. The expression “the bacterium” or “a bacterium” should be understood to include also reference to the plural.

The term “strain”, as used herein, refers to variants of a bacterial B. hyodysenteriae species that can be distinguished by one or more characteristics, such as ribosomal RNA sequence variation, DNA polymorphisms, serological typing, or toxin production, from other variants within that species.

The term “reference strain”, as used herein, refers to a specific strain that is widely used within the scientific community or adopted by formally recognized institutions, particularly the B. hyodysenteriae reference strain is the B. hyodysenteriae strain WA1.

The term “reference sequence”, as used herein, refers to a nucleotide or amino acid sequence from the Brachyspira spp. reference strain, particularly from B. hyodysenteriae strain WA1, to which a particular nucleotide or amino acid sequence can be compared to when identifying the sequence identity between said particular sequence and the reference sequence. The reference sequence can be a nucleotide sequence from any part of the genome, including extrachromosomal elements, or a sequence of a protein encoded in any part of the genome, including extrachromosomal elements. The reference sequence of strain WA1 is identified by the accession number NC_012225.1 in the GenBank database.

The term “plasmid pBH”, as used herein, refers to the circular plasmid of about 36 kilobase (kb) present in some B. hyodysenteriae strains which comprises an rfbBADC operon. This operon presents a gene arrangement unique to B. hyodysenteriae, and it is predicted to be involved in lipooligosaccharide (LOS) biosynthesis. In the particular case of the B. hyodysenteriae reference strain WA1 the plasmid pBH is known as pBHWA1 and has 35,940 base pairs. The plasmid of the WA1 reference strain is identified by the accession number NC_012226.1 in the GenBank database. The genes rfbA, rfbB, rfbC, rfbD form an operon on the plasmid pBH of 36 kb in the order rfbBADC in the B. hyodysenteriae reference strain WA1 (taxonomy ID 565034 in the NCBI Taxonomy Browser). This operon rfbBADC groups the genes related to the lipooligosaccharide synthesis, and also groups 4 of the 6 rfb genes present in the plasmid. The term plasmid pBH, however, is not restricted to this particular plasmid, but it refers to any plasmid from any B. hyodysenteriae strain of about 36 kb comprising the operon rfbBADC.

The term “sequence identity” means that sequences are identical (i.e., on a nucleotide-by-nucleotide basis for nucleic acids or amino acid-by-amino acid basis for polypeptides) over a window of comparison. The percentage of sequence identity is calculated by comparing two optimally aligned sequences over the comparison window, determining the number of positions at which the identical nucleotides or amino acids occurs in both sequences to yield the number of matched positions, dividing the number of matched positions by the total number of positions in the comparison window, and multiplying the result by 100 to yield the percentage of sequence identity. Methods to calculate sequence identity are known to those of skill in the art and described in further detail below.

The term “substantially identical” means that two nucleotide or amino acid sequences share at least 60% sequence identity, at least 70%, at least 75%, at least 80%, at least 85%, at least 90%, at least 95%, at least 96%, at least 97%, at least 98, or at least 99% sequence identity over a region of at least 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 nucleotides or amino acids. In a particular embodiment, “substantially identical” means that two nucleotide or amino acid sequences share at least 60% sequence identity, at least 70%, at least 75%, at least 80%, at least 85%, at least 90%, at least 95%, at least 96%, at least 97%, at least 98, or at least 99% sequence identity over the whole length of one of the sequences, or over the whole length of both sequences.

As used herein, the term “isolated” means that the bacterium has been removed from its natural and original environment. The term “isolated” does not necessarily reflect the extent to which the bacterium has been purified.

As used herein, the term “inactivated” refers to a dead/killed or inactivated cell of a microorganism, in this case a bacterium from the species B. hyodysenteriae, which is no longer capable growth on a plate having medium specific for said microorganism, and it also encompasses lysates, fractions or extracts of the microorganism. The inactivation of a microorganism refers to any process by which a viable cell is converted to a non-viable cell. The terms “viable”, “alive” or “viability”, as used herein, refer to the ability of a cell to maintain itself or recover its potentialities and survive until they are able to divide to generate a bacterial progeny. Thus, a cell that is viable or alive indicates that the cell is able to survive and divide; conversely, a cell that is non-viable indicates that the cell is not able to survive and divide. It will be appreciated that non-viable cells include dead/killed cells.

Viability can be determined by a number of assays that are conventional in the art. These include cytolysis or membrane leakage assays, such as the lactate dehydrogenase assay, the propidium iodide assay, the trypan blue assay and the 7-aminoactinomycin D assay, as well as genomic and proteomic assays that test the activation of stress pathways using DNA microarrays and protein chips. Viability can also be determined by checking the absence of cells after their culture in an appropriate culture medium. Therefore, an inactivated B. hyodysenteriae is different to an attenuated or live attenuated B. hyodysenteriae, wherein the cells are alive and viable.

The bacterium from the species B. hyodysenteriae can be inactivated by any suitable means used for inactivating microorganisms and known by the skilled person, either by physical or chemical methods among others, for example, formaldehyde inactivation, microwave inactivation, pressure inactivation, acid inactivation, base inactivation, alcohol inactivation, peroxide inactivation, irradiation and thermal inactivation. In a particular embodiment, the bacterium from the species B. hyodysenteriae is inactivated by formaldehyde. In a particular embodiment, formaldehyde inactivation is performed by incubating the bacteria with formaldehyde at 0.01%-1.0% (v/v), for example at 0.05%-0.8% (v/v), at 0.1%-0.5% (v/v), at 0.2% (v/v). In a particular embodiment, formaldehyde inactivation is performed by incubating the bacteria with formaldehyde during 6 hours to 7 days, for example during 8 hours to 5 days, during 10 hours to 2 days, during 12 hours to 36 hours, during 24 hours. In a particular embodiment, formaldehyde inactivation is performed by incubating the bacteria with formaldehyde at 25° C. to 40° C., at 28° C. to 39° C., at 30° C. to 38° C., at 35° C. to 37° C., at 37° C. In a more particular embodiment, formaldehyde inactivation is performed by incubating the bacteria with formaldehyde at 0.2% (v/v) during 24 hours at 37° C.

The term “fucT”, “fucT gene” or “gene on the locus BHWA1_02689 of the B. hyodysenteriae plasmid identified by the GenBank accession number NC_012226.1” refers to a gene that encodes the protein alpha-1,2-fucosyltransferase and that can be found on the plasmid pBH of about 36 kb of B. hyodysenteriae. In a particular embodiment, the gene fucT refers to the gene of the WA1 reference strain of B. hyodysenteriae. The fucT gene of the WA1 reference strain of B. hyodysenteriae can be found on the locus BHWA1_02689, and has a sequence corresponding to positions 26872 to 28743 in the sequence of the plasmid pBHWA1 identified in the GenBank database by the accession number NC_012226.1 (4 Apr. 2020). The fucT gene of the strain of B. hyodysenteriae WA1 has the sequence of SEQ ID NO: 5.

In another particular embodiment, the bacterium from the species B. hyodysenteriae comprises a variant of the fucT gene of any strain of B. hyodysenteriae.

The term “variant”, as used herein, applies to genes of B. hyodysenteriae, refers to variants of said genes that appear in other strains of B. hyodysenteriae or to artificial variants of said genes, that is, variants that cannot be naturally found in B. hyodysenteriae. Variants of the same gene in different strains of the same species or artificial variants may have insertions, deletions or substitutions of one or more base pairs. Variants of the same gene in different strains or artificial variants will have a certain degree of sequence identity, for example, at least 60%, at least 65%, at least 70%, at least 75%, at least 80%, at least 85%, at least 90%, at least 95%, at least 98%, at least 99% or higher sequence identity. The degree of identity between two polynucleotides may be determined using computer algorithms and methods which are widely known to those skilled in the art. The identity between two polynucleotides is preferentially determined using BLASTP algorithm [BLASTManual, Altschul, S. et al., NCBI NLM NIH Bethesda, Md. 20894, Altschul, S., et al., J. Mol. Biol. 215: 403-410 (1990)]. In a preferred embodiment, the sequence identity is determined throughout the whole length of the sequence of the gene from the WA1 reference strain of B. hyodysenteriae, or throughout the whole length of the variant or of both.

In a particular embodiment, the variant of the fucT gene has at least 60%, at least 65%, at least 70%, at least 75%, at least 80%, at least 85%, at least 90%, at least 95%, at least 96%, at least 97%, at least 98%, at least 99% or higher sequence identity with the fucT gene.

In a particular embodiment, the variant of the fucT gene comprises or consist of the sequence selected from the group consisting of SEQ ID NO: 17-31.
Strain B-7493 comprises the variant of the fucT gene of SEQ ID NO: 18.
Strain B-7500 comprises the variant of the fucT gene of SEQ ID NO: 30.
Strains B-7482 and B-7484 comprise the variant of the fucT gene of SEQ ID NO: 25.
Strain B-7494 comprises the variant of the fucT gene of SEQ ID NO: 29.
Strain B-8397 comprises the variant of the fucT gene of SEQ ID NO: 22.

In a particular embodiment, the variant of the fucT gene maintains the same function of the fucT gene when the variant replaces the fucT gene on locus BHWA1_02689 from the WA1 reference strain.

In a particular embodiment, the variant of the gene on the locus BHWA1_02689 from the WA1 reference strain that maintains the same function encodes a protein which is an alpha-1,2-fucosyltransferase.

As shown in examples 2-5, the administration of an immunogenic or vaccine composition comprising an isolated and inactivated B. hyodysenteriae bacterium comprising a variant of the fucT gene is able to reduce the clinical signs of swine dysentery when administered to pigs that are subsequently infected with B. hyodysenteriae. Particularly, immunogenic or vaccine compositions comprising an isolated and inactivated B. hyodysenteriae bacterium comprising a variant of the fucT gene reduce the diarrhea score, intestinal lesion score and average daily weight loss and increase survival rate in the vaccinated pigs infected with B. hyodysenteriae. Therefore, in a particular embodiment, the administration of an immunogenic or vaccine composition comprising an isolated and inactivated B. hyodysenteriae bacterium comprising a variant of the fucT gene induces at least 50%, at least 60%, at least 70%, at least 80%, at least 90%, at least 95%, at least 98%, at least 99%, at least 100% or higher of the reduction in the clinical signs of swine dysentery, particularly diarrhea score, intestinal lesion score and/or average daily weight loss, and/or results in a survival rate of at least 50%, at least 60%, at least 70%, at least 80%, at least 90%, at least 95%, at least 98%, at least 99%, at least 100% or higher, than the immunogenic or vaccine composition comprising an isolated and inactivated B. hyodysenteriae bacterium comprising a variant of the fucT gene of sequence SEQ ID NO: 18, 30, 25, 29 and 22.

The term “diarrhea score” refers to a parameter that measures the degree of diarrhea. The diarrhea score can be determined by methods know by the skilled person, for example, the methods described by Burrough E R et al., J Vet Diagn Invest. 2012, 24(6): 1025-34 and by Hansen C F et al., Br J Nutr. 2011, 106(10): 1506-13. In a particular embodiment, the diarrhea score is determined with the following scoring system:
normal stool (no diarrhea): 0 points
pasty stool (consistency of cement, mild diarrhea): 1 point
liquid stool with fecal content (moderate diarrhea): 2 points
watery stool (severe diarrhea, does not look like stool): 3 points
presence of mucus: 0.5 points
presence of blood: 0.5 points

In a particular embodiment, the vaccine composition of the invention or the vaccine composition obtained by the first method of the invention induces a reduction in the diarrhea score after 1 to 200 days post-vaccination, for example after 2 to 190 days post-vaccination, after 3 to 180 days post-vaccination, after 4 to 170 days post-vaccination, after 5 to 150 days post-vaccination, after 6 to 130 days post-vaccination, after 7 to 100 days post-vaccination, after 10 to 75 days post-vaccination, after 12 to 50 days post-vaccination, after 15 to 25 days post-vaccination, after 20 days post-vaccination, in subjects suffering from an infection by a bacterium from the species B. hyodysenteriae. The term “reduction in the diarrhea score” refers to any reduction compared with non-vaccinated control subjects, for example, a reduction of at least 5%, at least 10%, at least 20%, at least 30%, at least 40%, at least 50%, at least 60%, at least 70%, at least 80%, at least 90%, at least 95%, at least 100% or higher.

The term “intestinal lesion score” refers to a parameter that measures the degree of lesion in the intestine of a subject. In a particular embodiment, the intestinal lesion score is determined with the following scoring system:
absence of lesion: 0 points
mild enteritis, lesions in specific areas, normal or pasty content: 1 point
moderate enteritis, extensive lesions, diarrheal content: 2 points
severe enteritis, severely damaged or atrophic intestine, watery/blood stools, fibrin: 3 points
very severe enteritis, totally atrophic intestine, completely ulcerated mucosa: 4 points.

In another particular embodiment, the intestinal lesion score is determined with the following scoring system:
absence of lesion: 0 points
mild enteritis, lesions in specific areas, normal or pasty content: 1 point
moderate enteritis, extensive lesions, diarrheal content: 2 points
severe enteritis, severely damaged or atrophic intestine, watery/blood stools, fibrin: 3 points.

In a particular embodiment, the vaccine composition of the invention or the vaccine composition obtained by the first method of the invention induces a reduction in the intestinal lesion score after 1 to 200 days post-vaccination, for example after 2 to 190 days post-vaccination, after 3 to 180 days post-vaccination, after 4 to 170 days post-vaccination, after 5 to 150 days post-vaccination, after 6 to 130 days post-vaccination, after 7 to 100 days post-vaccination, after 10 to 75 days post-vaccination, after 12 to 50 days post-vaccination, after 15 to 25 days post-vaccination, after 20 days post-vaccination, in subjects suffering from an infection by a bacterium from the species Brachyspira hyodysenteriae. The term “reduction in the intestinal lesion score” refers to any reduction compared with non-vaccinated control subjects, for example, a reduction of at least 5%, at least 10%, at least 20%, at least 30%, at least 40%, at least 50%, at least 60%, at least 70%, at least 80%, at least 90%, at least 95%, at least 100% or higher.

The term “daily weight loss”, as used herein, refers to the average weight the subject loss per day during a period of time after being infected with a bacterium of the species B. hyodysenteriae. In a particular embodiment, the vaccine composition of the invention or the vaccine composition obtained by the first method of the invention induces a reduction in the daily weight loss after 1 to 200 days post-vaccination, for example after 2 to 190 days post-vaccination, after 3 to 180 days post-vaccination, after 4 to 170 days post-vaccination, after 5 to 150 days post-vaccination, after 6 to 130 days post-vaccination, after 7 to 100 days post-vaccination, after 10 to 75 days post-vaccination, after 12 to 50 days post-vaccination, after 15 to 25 days post-vaccination, after 20 days post-vaccination, in subjects suffering from an infection by a bacterium from the species Brachyspira hyodysenteriae. The term “reduction in the daily weight loss” refers to any reduction compared with non-vaccinated control subjects, for example, a reduction of at least 5%, at least 10%, at least 20%, at least 30%, at least 40%, at least 50%, at least 60%, at least 70%, at least 80%, at least 90%, at least 95%, at least 100% or higher.

The term “survival rate”, as used herein, refers to the percentage of subjects diagnosed with a disease, such as a subject with an infection by a bacterium of the species Brachyspira hyodysenteriae, that are alive after a certain period of time. In a particular embodiment, the vaccine composition of the invention or the vaccine composition obtained by the first method of the invention induces an increase in the survival rate after 1 to 200 days post-vaccination, for example after 2 to 190 days post-vaccination, after 3 to 180 days post-vaccination, after 4 to 170 days post-vaccination, after 5 to 150 days post-vaccination, after 6 to 130 days post-vaccination, after 7 to 100 days post-vaccination, after 10 to 75 days post-vaccination, after 12 to 50 days post-vaccination, after 15 to 25 days post-vaccination, after 20 days post-vaccination, in subjects suffering from an infection by a bacterium from the species Brachyspira hyodysenteriae. The term “increased in the survival rate” refers to any increased compared with non-vaccinated control subjects, for example, an increase of at least 5%, at least 10%, at least 20%, at least 30%, at least 40%, at least 50%, at least 60%, at least 70%, at least 80%, at least 90%, at least 95%, at least 100% or higher.

In a particular embodiment of the immunogenic or vaccine composition of the invention, the fucT gene or the variant thereof is comprised in a gene cluster, and the bacterium is a S1 or a S2 bacterium, wherein
the gene cluster of the S1 bacterium comprises the following genes: the gene on the locus BHWA1_02695 (bcsA) of the B. hyodysenteriae plasmid pBHWA1 identified by the GenBank accession number NC_012226.1 or a variant of said gene of any strain of B. hyodysenteriae, the rfbC gene, the rfbD gene, the rfbA gene, the rfbB gene, the gene on the locus BHWA1_02690 (rgpF) of the B. hyodysenteriae plasmid pBHWA1 identified by the GenBank accession number NC_012226.1 or a variant of said gene of any strain of B. hyodysenteriae and the fucT gene or a variant of said gene of any strain and
the gene cluster of the S2 bacterium comprises the following genes: the gene on the locus BHWA1_02695 (bcsA) of the B. hyodysenteriae plasmid pBHWA1 identified by the GenBank accession number NC_012226.1 or a variant of said gene of any strain of B. hyodysenteriae, the rfbC gene, the rfbD gene, the rfbA gene, a gene having the sequence of SEQ ID NO: 8 (adoM) or a variant of said gene of any strain of B. hyodysenteriae, the rfbB gene, the gene on the locus BHWA1_02690 (rgpF) of the B. hyodysenteriae plasmid pBHWA1 identified by the GenBank accession number NC_012226.1 or a variant of said gene of any strain of B. hyodysenteriae and the fucT gene or a variant of said gene of any strain of B. hyodysenteriae.

The term “gene cluster” as used herein, refers to a set of closely linked genes that encode functionally-related products and that usually grouped together in the genome. In the context of the composition of the invention, the gene cluster refers to a cluster comprising the rfbBADC operon and other related genes.

The term “operon”, as used herein, refers to a genetic unit that controls gene expression in prokaryotes. An operon typically comprises one or more genes that encode one or more polypeptide(s) or RNA(s) and the adjacent regulatory region (or regions) that controls the transcription of the genes. The regulatory region typically comprises a promoter and an operator. The coding region of a prokaryotic gene is historically termed a “cistron.” Operons that contain multiple cistrons are termed “polycistronic.” The genes in a polycistronic operon are typically related in function and are typically co-transcribed as a single unit and expressed in a coordinated manner.

The term “rfb operon” or “rfbAD operon” or “rfbBADC” operon, as used herein, refers to an operon that groups several genes related to the lipooligosaccharide synthesis, including 4 of the 6 rfb genes present in the plasmid pBH of B. hyodysenteriae. The term “rfb genes” refers to 6 genes, named rfbA, rfbB, rfbC, rfbD, rfbE and rfbF. The rfb operon of B. hyodysenteriae includes the genes rfbA, rfbB, rfbC and rfbD and is described by Bellgard et al., PLoS ONE 2009, 4(3) e4641.

The gene cluster comprising the rfb operon of B. hyodysenteriae also comprises the genes bcsA, rgpF and optionally adoM and fucT. The structure of the gene cluster is what differentiates S1, S2, S3 and S0 B. hyodysenteriae bacteria as explained before.

Therefore, in a particular embodiment, the gene cluster comprising the rfb operon of B. hyodysenteriae comprises or consist of the genes bcsA, rfbC, rfbD, rfbA, rfbB, rgpF and fucT (gene cluster from S1 bacteria). In a more particular embodiment, the gene cluster comprising the rfb operon comprises or consists of, from the 5′ to the 3′ end, the genes bcsA, rfbC, rfbD, rfbA, rfbB, rgpF and fucT.

In another particular embodiment, the gene cluster comprising the rfb operon of B. hyodysenteriae comprises or consists of the genes bcsA, rfbC, rfbD, rfbA, adoM, rfbB, rgpF and fucT (gene cluster from S2 bacteria). In a more particular embodiment, the gene cluster comprising the rfb operon comprises or consists of, from the 5′ to the 3′ end, the genes bcsA, rfbC, rfbD, rfbA, adoM, rfbB, rgpF and fucT.

In another particular embodiment, the gene cluster comprising the rfb operon of B. hyodysenteriae comprises or consists of the genes bcsA, rfbC, rfbD, rfbA, adoM, rfbB and rgpF and does not comprise the gene fucT (gene cluster from S3 bacteria). In a more particular embodiment, the gene cluster comprising the rfb operon comprises or consists of, from the 5′ to the 3′ end, the genes bcsA, rfbC, rfbD, rfbA, AdoM, rfbB and rgpF.

The term “S1 bacterium”, as used herein, refers to a strain or a bacterium from the species B. hyodysenteriae comprising a gene cluster comprising or consisting of the genes bcsA, rfbC, rfbD, rfbA, rfbB, rgpF and fucT, or variants of said genes of any strain of B. hyodysenteriae. In a particular embodiment, the S1 bacterium comprises a gene cluster comprising or consisting of, from the 5′ to the 3′ end, the genes bcsA, rfbC, rfbD, rfbA, rfbB, rgpF and fucT. In a more particular embodiment, the S1 bacterium does not comprise the gene adoM.

The term “S2 bacterium”, as used herein, refers to a strain or a bacterium from the species B. hyodysenteriae comprising a gene cluster comprising or consisting of the genes bcsA, rfbC, rfbD, rfbA, adoM, rfbB, rgpF and fucT. In a particular embodiment, the S2 bacterium comprises a gene cluster comprising or consisting of, from the 5′ to the 3′ end, the genes bcsA, rfbC, rfbD, rfbA, adoM, rfbB, rgpF and fucT.

The term “S3 bacterium”, as used herein, refers to a strain or a bacterium from the species B. hyodysenteriae comprising a gene cluster comprising or consisting of the genes bcsA, rfbC, rfbD, rfbA, adoM, rfbB and rgpF. In a particular embodiment, the S3 bacterium comprises a gene cluster comprising or consisting of, from the 5′ to the 3′ end, the genes bcsA, rfbC, rfbD, rfbA, adoM, rfbB and rgpF. In a more particular embodiment, the S3 bacterium does not comprise the gene fucT.

In a particular embodiment, the S1, S2 and S3 bacterium comprises the gene cluster in the plasmid pBH.

In a particular embodiment, the S1 and S2 bacterium comprises the gene cluster comprising the fucT gene in the plasmid pBH.

The term “S0 bacterium”, as used herein, refers to a strain or a bacterium from the species B. hyodysenteriae that does not comprise the plasmid pBH. In a particular embodiment, the S0 bacterium does not comprise an rfb operon. In a more particular embodiment, the S0 bacterium does not comprise any of the genes bcsA, rfbC, rfbD, rfbA, rfbB, rgpF, fucT and adoM.

In a particular embodiment, the immunogenic or vaccine composition comprises a S1 bacterium, and the S1 bacterium comprises gene cluster comprising or consisting of, from the 5′ to the 3′ end, the genes bcsA, rfbC, rfbD, rfbA, rfbB, rgpF and fucT.

In another particular embodiment, the immunogenic or vaccine composition comprises a S2 bacterium, and the S2 bacterium comprises a gene cluster comprising or consisting of, from the 5′ to the 3′ end, the genes bcsA, rfbC, rfbD, rfbA, adoM, rfbB, rgpF and fucT.

The term “rfbA” refers to a gene that encodes the protein glucose-1-phosphate thymidylyltransferase and that can be found on the plasmid pBH of B. hyodysenteriae. The rfbA gene can be from a bacterium from any strain of B. hyodysenteriae. In a particular embodiment, the rfbA gene refers to the gene of the WA1 reference strain of B. hyodysenteriae. The rfbA gene of the WA1 reference strain of B. hyodysenteriae can be found on the locus BHWA1_02692, and has a sequence corresponding to positions 30900 to 31763 in the sequence of the plasmid pBHWA1 identified in the GenBank database by the accession number NC_012226.1 (4 Apr. 2020). The rfbA gene of the reference strain of B. hyodysenteriae WA1 has the sequence of SEQ ID NO: 1.

In a particular embodiment, the rfbA gene is the gene of the reference strain WA1 of B. hyodysenteriae WA1 or a variant of said gene of any strain of B. hyodysenteriae.

In a particular embodiment, the variant of the rfbA gene from the WA1 reference strain has at least 60%, at least 65%, at least 70%, at least 75%, at least 80%, at least 85%, at least 90%, at least 95%, at least, 96%, at least 97%, at least 98%, at least 99% or higher sequence identity with the rfbA gene from the WA1 reference strain.

In a particular embodiment, the variant of the rfbA gene from the WA1 reference strain comprises or consist of a sequence selected from the group consisting of SEQ ID NO: 32-44.
Strain B-7482 comprises the variant of the rfbA gene of SEQ ID NO: 32.
Strains B-7484 and B-8397 comprise the variant of the rfbA gene of SEQ ID NO: 34.
Strain B-7494 comprises the variant of the rfbA gene of SEQ ID NO: 44.
Strain B-7500 comprises the variant of the rfbA gene of SEQ ID NO: 39.
Strain B-8295 comprises the variant of the rfbA gene of SEQ ID NO: 41.
Strain B-7493 comprises the variant of the rfbA gene of SEQ ID NO: 42.

In a particular embodiment, the variant of the rfbA gene from the WA1 reference strain maintains the same function of the rfbA gene from the WA1 reference strain. In a particular embodiment, the expression “maintains the same function” means that the variant is capable of performing the same function as the rfbA gene of the WA1 reference strain when forming part of the rfb operon instead of the rfbA gene of the B. hyodysenteriae reference strain WA1 and together with the rest of the genes of the operon. In a more particular embodiment, the variant maintains the function of the rfbA gene when it is found in a gene cluster comprising or consisting of
the genes bcsA, rfbC, rfbD, rfbA, rfbB, rgpF and fucT, more particularly, from the 5′ to the 3′ end, the genes bcsA, rfbC, rfbD, rfbA, rfbB, rgpF and fucT (gene cluster from S1 bacterium),
the genes bcsA, rfbC, rfbD, rfbA, adoM, rfbB, rgpF and fucT, more particularly, from the 5′ to the 3′ end, the genes bcsA, rfbC, rfbD, rfbA, adoM, rfbB, rgpF and fucT (gene cluster from S2 bacterium);
the genes bcsA, rfbC rfbD, rfbA, adoM, rfbB and rbpF, wherein the operon does not comprise the gene fucT; more particularly, from the 5′ to the 3′ end, the genes bcsA, rfbC, rfbD, rfbA, AdoM, rfbB and rgpF (gene cluster from S3 bacterium).

In a particular embodiment, the administration of an immunogenic or vaccine composition comprising an isolated and inactivated B. hyodysenteriae bacterium comprising a variant of the rfbA gene induces at least 50%, at least 60%, at least 70%, at least 80%, at least 90%, at least 95%, at least 98%, at least 99%, at least 100% or higher of the reduction in the clinical signs of swine dysentery, particularly diarrhea score, intestinal lesion score and/or average daily weight loss, and/or results in a survival rate of at least 50%, at least 60%, at least 70%, at least 80%, at least 90%, at least 95%, at least 98%, at least 99%, at least 100% or higher, than the immunogenic or vaccine composition comprising an isolated and inactivated B. hyodysenteriae bacterium comprising a variant of rfbA comprising or consisting of the sequence of SEQ ID NO: 32, 34, 44, 39, 41 and 42.

In a particular embodiment, the variant of the rfbA gene from the WA1 reference strain encodes a protein which is a glucose-1-phosphate thymidylyltransferase.

The term “rfbB” refers to a gene that encodes the protein dTDP-glucose 4,6-dehydratase and that can be found on the plasmid pBH of B. hyodysenteriae. The rfbB gene can be from a bacterium from any strain of B. hyodysenteriae. In a particular embodiment, the gene rfbB refers to the gene of the WA1 reference strain of B. hyodysenteriae. The gene rfbB of the WA1 reference strain of B. hyodysenteriae can be found on the locus BHWA1_02691, and has a sequence corresponding to positions 29816 to 30886 in the sequence of the plasmid pBHWA1 identified in the GenBank database by the accession number NC_012226.1 (4 Apr. 2020). The rfbB gene of the reference strain of B. hyodysenteriae WA1 has the sequence of SEQ ID NO: 2.

In a particular embodiment, the rfbB gene is the gene of the WA1 reference strain of B. hyodysenteriae or a variant of said gene of any strain of B. hyodysenteriae.

In a particular embodiment, the variant of the rfbB gene from the WA1 reference strain has at least 60%, at least 65%, at least 70%, at least 75%, at least 80%, at least 85%, at least 90%, at least 95%, at least 96%, at least 97%, at least 98%, at least 99% or higher sequence identity with the rfbB gene from the WA1 reference strain.

In a particular embodiment, the variant of the rfbB gene from the WA1 reference strain comprises or consist of a sequence selected from the group consisting of SEQ ID NO: 45-54.
Strain B-7482 comprises a variant of the rfbB gene of sequence SEQ ID NO: 45.
Strain B-7493 comprises a variant of the rfbB gene of sequence SEQ ID NO: 46.
Strain B-7500 comprises a variant of the rfbB gene of sequence SEQ ID NO: 49.
Strain B-8295 comprises a variant of the rfbB gene of sequence SEQ ID NO: 52.
Strains B-7484, B-7494 and B-8397 comprise a variant of the rfbB gene of sequence SEQ ID NO: 53.

In a particular embodiment, the variant of the rfbB gene from the reference WA1 strain maintains the same function of the rfbB gene from the WA1 strain. In a particular embodiment, the variant is capable of performing the same function as the rfbB gene of the WA1 reference strain when forming part of the rfb operon instead of the rfbB gene of the B. hyodysenteriae reference strain WA1 and together with the rest of the genes of the operon. In a more particular embodiment, the variant maintains the function of the rfbB gene when it is found in a gene cluster comprising or consisting of the genes previously indicated for the gene cluster comprising the rfb operon from S1, S2 or S3 bacterium.

In a particular embodiment, the administration of an immunogenic or vaccine composition comprising an isolated and inactivated B. hyodysenteriae bacterium comprising a variant of the rfbB gene induces at least 50%, at least 60%, at least 70%, at least 80%, at least 90%, at least 95%, at least 98%, at least 99%, at least 100% or higher of the reduction in the clinical signs of swine dysentery, particularly diarrhea score, intestinal lesion score and/or average daily weight loss, and/or results in a survival rate of at least 50%, at least 60%, at least 70%, at least 80%, at least 90%, at least 95%, at least 98%, at least 99%, at least 100% or higher, than the immunogenic or vaccine composition comprising an isolated and inactivated B. hyodysenteriae bacterium comprising a variant of rfbB comprising or consisting of the sequence of SEQ ID NO: 45, 46, 49, 52 and 53.

In a particular embodiment, the variant of the rfbB gene from the WA1 reference strain encodes a protein which is a dTDP-glucose 4,6-dehydratase.

The term “rfbC” refers to a gene that encodes the protein dTDP-4-dehydrorhamnose 3,5-epimerase and that can be found on the plasmid pBH of B. hyodysenteriae. The rfbC gene can be from a bacterium from any strain of B. hyodysenteriae. In a particular embodiment, the gene rfbC refers to the gene of the WA1 reference strain of B. hyodysenteriae. The gene rfbC of the WA1 reference strain of B. hyodysenteriae can be found on the locus BHWA1_02694, and has a sequence corresponding to positions 32648 to 33226 in the sequence of the plasmid pBHWA1 identified in the GenBank database by the accession number NC_012226.1 (4 Apr. 2020). The rfbC gene of the reference strain of B. hyodysenteriae WA1 has the sequence of SEQ ID NO: 3.

In a particular embodiment, the rfbC gene is the gene of the WA1 reference strain of B. hyodysenteriae or a variant of said gene of any strain of B. hyodysenteriae.

In a particular embodiment, the variant of the rfbC gene from the WA1 reference strain has at least 60%, at least 65%, at least 70%, at least 75%, at least 80%, at least 85%, at least 90%, at least 95%, at least 96%, at least 97%, at least 98%, at least 99% or higher sequence identity with the rfbC gene from the WA1 reference strain.

In a particular embodiment, the variant of the rfbC gene from the WA1 reference strain comprises or consist of a sequence selected from the group consisting of SEQ ID NO: 55-61.
Strains B-7482, B-7500, B-7484, B-7494 and B-8397 comprise the rfbC gene of sequence SEQ ID NO: 3.
Strain B-8295 comprises a variant of the rfbC gene of sequence SEQ ID NO: 61.
Strain B-7493 comprises a variant of the rfbC gene of sequence SEQ ID NO: 57.

In a particular embodiment, the variant of the rfbC gene from the WA1 reference strain that maintains the same function encodes a protein which is a dTDP-4-dehydrorhamnose 3,5-epimerase.

In a particular embodiment, the variant of the rfbC gene from the WA1 reference strain maintains the same function of the rfbC gene from the WA1 reference strain. In a particular embodiment, the variant is capable of performing the same function as the rfbC gene of the WA1 reference strain when forming part of the rfb operon instead of the rfbC gene of the B. hyodysenteriae reference strain WA1 and together with the rest of the genes of the operon. In a more particular embodiment, the variant maintains the function of the rfbC gene when it is found in a gene cluster comprising or consisting of the genes previously indicated for the gene cluster comprising the rfb operon from S1, S2 or S3 bacterium.

In a particular embodiment, the administration of an immunogenic or vaccine composition comprising an isolated and inactivated B. hyodysenteriae bacterium comprising a variant of the rfbC gene induces at least 50%, at least 60%, at least 70%, at least 80%, at least 90%, at least 95%, at least 98%, at least 99%, at least 100% or higher of the reduction in the clinical signs of swine dysentery, particularly diarrhea score, intestinal lesion score and/or average daily weight loss, and/or results in a survival rate of at least 50%, at least 60%, at least 70%, at least 80%, at least 90%, at least 95%, at least 98%, at least 99%, at least 100% or higher, than the immunogenic or vaccine composition comprising an isolated and inactivated B. hyodysenteriae bacterium comprising the rfbC gene of sequence SEQ ID NO: 3 or a variant of rfbC comprising or consisting of the sequence of SEQ ID NO: 61 and 57.

The term “rfbD” refers to a gene that encodes the protein dTDP-4-dehydrorhamnose reductase and that can be found on the plasmid pBH of B. hyodysenteriae. The rfbD gene can be from a bacterium from any strain of B. hyodysenteriae. In a particular embodiment, the gene rfbD refers to the gene of the WA1 reference strain of B. hyodysenteriae. The gene rfbD of the WA1 reference strain of B. hyodysenteriae can be found on the locus BHWA1_02693, and has a sequence corresponding to positions 31782 to 32642 in the sequence of the plasmid pBHWA1 identified in the GenBank database by the accession number NC_012226.1 (4 Apr. 2020). The rfbD gene of the strain of B. hyodysenteriae WA1 has the sequence of SEQ ID NO: 4.

In a particular embodiment, the rfbD gene is the gene of the WA1 reference strain of B. hyodysenteriae or a variant of said gene of any strain of B. hyodysenteriae.

In a particular embodiment, the variant of the rfbD gene from the WA1 reference strain has at least 60%, at least 65%, at least 70%, at least 75%, at least 80%, at least 85%, at least 90%, at least 95%, at least 96%, at least 97%, at least 98%, at least 99% or higher sequence identity with the rfbD gene from the WA1 reference strain.

In a particular embodiment, the variant of the rfbD gene from the WA1 reference strain comprises or consist of a sequence selected from the group consisting of SEQ ID NO: 62-68.
Strains B-7482 and B-8295 comprise the rfbD gene of sequence SEQ ID NO: 4.
Strains B-7500, B-7484, B-7494 and B-8397 comprise a variant of the rfbD gene of sequence SEQ ID NO: 62.
Strain B-7493 comprises a variant of the rfbD gene of sequence SEQ ID NO: 68.

In a particular embodiment, the variant of the rfbD gene from the WA1 reference strain that maintains the same function encodes a protein which is a dTDP-4-dehydrorhamnose 3,5-epimerase.

In a particular embodiment, the variant of the rfbD gene from the WA1 reference strain maintains the same function of the rfbD gene from the WA1 reference strain. In a particular embodiment, the variant is capable of performing the same function as the rfbD gene of the WA1 reference strain when forming part of the rfb operon instead of the rfbD gene of the B. hyodysenteriae reference strain WA1 and together with the rest of the genes of the operon. In a more particular embodiment, the variant maintains the function of the rfbD gene when it is found in a gene cluster comprising or consisting of the genes previously indicated for the gene cluster comprising the rfb operon from S1, S2 or S3 bacterium.

In a particular embodiment, the administration of an immunogenic or vaccine composition comprising an isolated and inactivated B. hyodysenteriae bacterium comprising a variant of the rfbD gene induces at least 50%, at least 60%, at least 70%, at least 80%, at least 90%, at least 95%, at least 98%, at least 99%, at least 100% or higher of the reduction in the clinical signs of swine dysentery, particularly diarrhea score, intestinal lesion score and/or average daily weight loss, and/or results in a survival rate of at least 50%, at least 60%, at least 70%, at least 80%, at least 90%, at least 95%, at least 98%, at least 99%, at least 100% or higher, than the immunogenic or vaccine composition comprising an isolated and inactivated B. hyodysenteriae bacterium comprising the rfbD gene of sequence SEQ ID NO: 4 or a variant of rfbD comprising or consisting of the sequence of SEQ ID NO: 62 and 68.

The term “rgpF” or “gene on the locus BHWA1_02690 of the B. hyodysenteriae plasmid identified by the GenBank accession number NC_012226.1” refers to a gene that encodes the lipopolysaccharide biosynthesis protein-like protein and that can be found on the plasmid pBH of B. hyodysenteriae. The rgpF gene can be from a bacterium from any strain of B. hyodysenteriae. In a particular embodiment, the rgpF gene refers to the gene of the WA1 reference strain of B. hyodysenteriae. The rgpF gene of the WA1 reference strain of B. hyodysenteriae can be found on the locus BHWA1_02690, and has a sequence corresponding to positions 28827 to 29807 in the sequence of the plasmid pBHWA1 identified in the GenBank database by the accession number NC_012226.1 (4 Apr. 2020). The rgpF gene of the reference strain of B. hyodysenteriae WA1 has the sequence of SEQ ID NO: 6.

In a particular embodiment, the rgpF gene is a variant of said gene of any strain of B. hyodysenteriae.

In a particular embodiment, the variant of the rgpF gene has at least 60%, at least 65%, at least 70%, at least 75%, at least 80%, at least 85%, at least 90%, at least 95%, at least 96%, at least 97%, at least 98%, at least 99% or higher sequence identity with the rgpF gene.

In a particular embodiment, the variant of the gene rgpF comprises or consist of a sequence selected from the group consisting of SEQ ID NO: 69-76.
Strains B-7482, B-8295, B-7484, B-7494 and B-8397 comprise a variant of the rgpF gene of sequence SEQ ID NO: 71.
Strain B-7493 comprises a variant of the rgpF gene of sequence SEQ ID NO: 74.
Strain B-7500 comprises a variant of the rgpF gene of sequence SEQ ID NO: 75.

In a particular embodiment, the variant of rgpF maintains the same function of the gene rgpF. In a particular embodiment, the variant is capable of performing the same function as the gene rgpF when forming part of the gene cluster comprising the rfb operon instead of the gene of the B. hyodysenteriae reference strain WA1 located on locus BHWA1_02690 and together with the rest of the genes of the gene cluster. In a more particular embodiment, the variant maintains the function of the gene rgpF when it is found in a gene cluster comprising or consisting of the genes previously indicated for a gene cluster comprising the rfb operon from S1, S2 or S3 bacterium.

In a particular embodiment, the administration of an immunogenic or vaccine composition comprising an isolated and inactivated B. hyodysenteriae bacterium comprising a variant of the rgpF gene induces at least 50%, at least 60%, at least 70%, at least 80%, at least 90%, at least 95%, at least 98%, at least 99%, at least 100% or higher of the reduction in the clinical signs of swine dysentery, particularly diarrhea score, intestinal lesion score and/or average daily weight loss, and/or results in a survival rate of at least 50%, at least 60%, at least 70%, at least 80%, at least 90%, at least 95%, at least 98%, at least 99%, at least 100% or higher, than the immunogenic or vaccine composition comprising an isolated and inactivated B. hyodysenteriae bacterium comprising a variant of rgpF comprising or consisting of the sequence of SEQ ID NO: 71, 74 and 75.

In a particular embodiment, the variant of the rgpF gene that maintains the same function as the rgpF gene encodes a lipopolysaccharide biosynthesis protein-like protein.

The term “bcsA” or “gene on the locus BHWA1_02695 of the B. hyodysenteriae plasmid identified by the GenBank accession number NC_012226.1” refers to a gene that encodes a glycosyltransferase family 2 protein and that can be found on the plasmid pBH of B. hyodysenteriae. The bcsA gene can be from a bacterium from any strain of B. hyodysenteriae. In a particular embodiment, the bcsA gene refers to the gene of the WA1 reference strain of B. hyodysenteriae. The bcsA gene of the WA1 reference strain of B. hyodysenteriae can be found on the locus BHWA1_02695 of the plasmid of WA1 reference strain of B. hyodysenteriae, and has a sequence corresponding to positions 33234 to 34421 in the sequence of the plasmid pBHWA1 identified in the GenBank database by the accession number NC_012226.1 (4 Apr. 2020). The bcsA gene can be from a bacterium from any strain of B. hyodysenteriae. In a particular embodiment, the gene bcsA refers to the gene of the WA1 reference strain of B. hyodysenteriae. The bcsA gene of the reference strain of B. hyodysenteriae WA1 has the sequence of SEQ ID NO: 7.

In a particular embodiment, the gene cluster comprises a variant of the bcsA gene of any strain of B. hyodysenteriae.

In a particular embodiment, the variant of the bcsA gene has at least 60%, at least 65%, at least 70%, at least 75%, at least 80%, at least 85%, at least 90%, at least 95%, at least 96%, at least 97%, at least 98%, at least 99% or higher sequence identity with the bcsA gene.

In a particular embodiment, the variant of the bcsA gene comprises or consist of a sequence selected from the group consisting of SEQ ID NO: 77-81.
Strain B-7500 comprises a variant of the bcsA gene of sequence SEQ ID NO: 77.
Strain B-7484 comprises a variant of the bcsA gene of sequence SEQ ID NO: 81.
Strains B-7482, B-7493, B-8295, B-7494 and B-8397 comprise the bcsA gene of sequence SEQ ID NO: 7.

In a particular embodiment, the variant of the bcsA gene maintains the same function of the gene bcsA. In a particular embodiment, the variant is capable of performing the same function as the gene bcsA when forming part of the gene cluster instead of the gene of the B. hyodysenteriae reference strain WA1 on the locus BHWA1_02695 and together with the rest of the genes of the gene cluster. In a more particular embodiment, the variant maintains the function of the gene on the locus BHWA1_02695 when it is found in gene cluster comprising or consisting of the genes previously indicated for the gene cluster comprising the rfb operon from S1, S2 or S3 bacterium.

In a particular embodiment, the variant of the bcsA gene that maintains the same function as the bcsA gene encodes a glycosyltransferase family 2 protein.

In a particular embodiment, the administration of an immunogenic or vaccine composition comprising an isolated and inactivated B. hyodysenteriae bacterium comprising a variant of the bcsA gene induces at least 50%, at least 60%, at least 70%, at least 80%, at least 90%, at least 95%, at least 98%, at least 99%, at least 100% or higher of the reduction in the clinical signs of swine dysentery, particularly diarrhea score, intestinal lesion score and/or average daily weight loss, and/or results in a survival rate of at least 50%, at least 60%, at least 70%, at least 80%, at least 90%, at least 95%, at least 98%, at least 99%, at least 100% or higher, than the immunogenic or vaccine composition comprising an isolated and inactivated B. hyodysenteriae bacterium comprising the bcsA gene of sequence SEQ ID NO: 7 or a variant of bcsA comprising or consisting of the sequence of SEQ ID NO: 77 or 81.

The term “gene having the sequence of SEQ ID NO: 8” or “adoM” refers to a gene that encodes a methyltransferase domain-containing protein. The adoM gene can be from a bacterium from any strain of B. hyodysenteriae.

In a particular embodiment, the gene cluster comprises a variant of the adoM gene of any strain of B. hyodysenteriae.

In a particular embodiment, the variant of the adoM gene has at least 60%, at least 65%, at least 70%, at least 75%, at least 80%, at least 85%, at least 90%, at least 95%, at least 96%, at least 97%, at least 98%, at least 99% or higher sequence identity with the sequence of the adoM gene.

In a particular embodiment, the variant of the adoM gene comprises or consist of the sequence of SEQ ID NO: 82.
Strains B-7484, B-7494 and B-8397 comprise the adoM gene of sequence SEQ ID NO: 8.

In a particular embodiment, the variant of the adoM gene maintains the same function of the gene adoM. In a particular embodiment, the variant is capable of performing the same function as the gene adoM when forming part of the gene cluster instead of the gene adoM of SEQ ID NO:8 and together with the rest of the genes of the gene cluster. In a more particular embodiment, the variant maintains the function of the adoM gene when it is found in gene cluster comprising or consisting of the genes previously indicated for the gene cluster comprising the rfb operon from S2 or S3 bacterium.

In a particular embodiment, the administration of an immunogenic or vaccine composition comprising an isolated and inactivated B. hyodysenteriae bacterium comprising a variant of the adoM gene induces at least 50%, at least 60%, at least 70%, at least 80%, at least 90%, at least 95%, at least 98%, at least 99%, at least 100% or higher of the reduction in the clinical signs of swine dysentery, particularly diarrhea score, intestinal lesion score and/or average daily weight loss, and/or results in a survival rate of at least 50%, at least 60%, at least 70%, at least 80%, at least 90%, at least 95%, at least 98%, at least 99%, at least 100% or higher, than the immunogenic or vaccine composition comprising an isolated and inactivated B. hyodysenteriae bacterium comprising a the adoM gene of sequence SEQ ID NO: 8.

In a particular embodiment, the variant of the adoM gene encodes a methyltransferase domain-containing protein.

In a particular embodiment, the S1 bacterium is a bacterium from a strain selected from the group consisting of B-7493, B-7500, B-8295, and B-7482.

The strain B-7500 corresponds to a strain of B. hyodysenteriae deposited by HIPRA SCIENTIFIC S.L.U. (Avda. La Selva, 135-17160, Amer, Girona, Spain) in the Leibniz-Institut DSMZ-Deutsche Sammlung von Mikroorganismen und Zellkulturen GmbH (Inhoffenstr. 7 B, D-38124 Braunschweig, Germany) under accession number DSM 33602 on Jul. 29, 2020.

The strain B-8295 corresponds to a strain of B. hyodysenteriae deposited by HIPRA SCIENTIFIC S.L.U. (Avda. La Selva, 135-17160, Amer, Girona, Spain) in the Leibniz-Institut DSMZ-Deutsche Sammlung von Mikroorganismen und Zellkulturen GmbH (Inhoffenstr. 7 B, D-38124 Braunschweig, Germany) under accession number DSM 33603 on Jul. 29, 2020.

In a particular embodiment, the S2 bacterium is a bacterium from a strain selected from the group consisting of B-7484, B-7494, and B-8397.

The strain B-8397 corresponds to a strain of B. hyodysenteriae deposited by HIPRA SCIENTIFIC S.L.U. (Avda. La Selva, 135-17160, Amer, Girona, Spain) in the Leibniz-Institut DSMZ-Deutsche Sammlung von Mikroorganismen und Zellkulturen GmbH (Inhoffenstr. 7 B, D-38124 Braunschweig, Germany) under accession number DSM 33604 on Jul. 29, 2020.

In a particular embodiment, the immunogenic or vaccine composition comprises or consists of at least two strains of a S1 bacterium. In a particular embodiment, the two strains are selected from the group consisting of B-7493, B-7500, B-8295 and B-7482.

In a more particular embodiment, the two strains are B-7500 and B-8295.

In another particular embodiment, the immunogenic or vaccine composition comprises at least two strains of a S2 bacterium. In a particular embodiment, the two strains are selected from the group consisting of B-8397, B-7484, and B-7494. In a more particular embodiment, the two strains are B-8397 and B-7484.

In another particular embodiment, the immunogenic or vaccine composition comprises at least two strains of a S1 bacterium and at least one strain of a S2 bacterium.

In a more particular embodiment, the immunogenic or vaccine composition comprises or consists of S1 strains B-7500 and B-8295 and S2 strain B-8397.

In another particular embodiment, the immunogenic or vaccine composition comprises at least one strain of a S1 bacterium and at least two strains of a S2 bacterium.

In another particular embodiment, the immunogenic or vaccine composition comprises or consists of at least two strains of a S1 bacterium and at least two strains of a S2 bacterium. In a more particular embodiment, the immunogenic or vaccine composition comprises or consists of the S1 strains B-7500 and B-8295 and the S2 strains B-8397 and B-7484.

In another particular embodiment, the immunogenic or vaccine composition comprises or consists of at least three strains of a S1 bacterium and at least two strains of a S2 bacterium. In a more particular embodiment, the immunogenic or vaccine composition comprises or consists of the S1 strains B-7500, B-8295 and B-7482 and the S2 strains B-8397 and B-7484.

In a particular embodiment the immunogenic or vaccine composition comprises or consists of:
the strain B-7500, deposited by in the Leibniz-Institut DSMZ-Deutsche Sammlung von Mikroorganismen und Zellkulturen GmbH under accession number DSM 33602,
the strain B-8295, deposited in the Leibniz-Institut DSMZ-Deutsche Sammlung von Mikroorganismen und Zellkulturen GmbH under accession number DSM 33603 and
the strain B-8397, deposited in the Leibniz-Institut DSMZ-Deutsche Sammlung von Mikroorganismen und Zellkulturen GmbH under accession number DSM 33604.

In a particular embodiment, the immunogenic or vaccine composition of the invention further comprises a veterinary acceptable excipient.

The term “veterinary acceptable” means that the component can be administered to a subject along with the immunogenic or vaccine composition of the invention without causing any undesirable biological effect or interacting in a deleterious manner with any of the other components of the composition. It is usually approved by a regulatory agency of a state or federal government or is included in the Eur. Ph. or the U.S. Pharmacopoeia or other generally recognized pharmacopoeia, including those that apply for use in animals, and more particularly in swine.

The term “excipient” refers to a vehicle, or diluent that is administered with the active ingredient and includes solvents, dispersion media, coatings, stabilizing agents, diluents, preservatives, antibacterial and antifungal agents, isotonic agents, adsorption-delaying agents, and the like. Such pharmaceutical excipients can be sterile liquids, such as water and oils, including those of petroleum, animal, vegetable or synthetic origin, such as peanut oil, soybean oil, mineral oil, sesame oil and similar. Water or saline aqueous solutions and aqueous dextrose and glycerol solutions, particularly for injectable solutions, are preferably used as vehicles. Suitable pharmaceutical vehicles are described in “Remington's Pharmaceutical Sciences” by E. W. Martin, 21st Edition, 2005; or “Handbook of Pharmaceutical Excipients”, Rowe C. R.; Paul J. S.; Marian E. Q., sixth Edition.

Suitable veterinary acceptable vehicles include, for example, water, salt solutions, alcohol, vegetable oils, polyethylene glycols, gelatin, lactose, amylose, magnesium stearate, talc, surfactants, silicic acid, viscous paraffin, perfume oil, monoglycerides and diglycerides of fatty acids.

In a particular embodiment, the immunogenic or vaccine composition further comprises an adjuvant. The term “adjuvant”, as used herein, refers to a substance which, when added to an immunogenic agent, non-specifically enhances or potentiates an immune response to the agent in a recipient host upon exposure to the mixture. Illustrative non-limitative examples of adjuvants that can be included in the immunogenic or vaccine composition of the invention include adjuvants formed by aluminum salts (alum), such as aluminum hydroxide, aluminum phosphate, aluminum sulfate, etc., formulations of oil-in-water or water-in-oil emulsions such as complete Freund's Adjuvant (CFA) as well as the incomplete Freund's Adjuvant (IFA); mineral gels; block copolymers, Avridine™, SEAM62, adjuvants formed by components of the bacterial cell wall such as adjuvants including liposaccharides (e.g., lipid A or Monophosphoryl Lipid A (MPLA), trehalose dimycolate (TDM), and components of the cell wall skeleton (CWS), heat shock proteins or the derivatives thereof, adjuvants derived from ADP-ribosylating bacterial toxins, which include diphtheria toxin (DT), pertussis toxin (PT), cholera toxin (CT), Escherichia coli heat-labile toxins (LT1 and LT2), Pseudomonas Endotoxin A and exotoxin, Bacillus cereus exoenzyme B, Bacillus sphaericus toxin, Clostridium botulinum toxins C2 and C3, Clostridium limosum exoenzyme as well as the toxins of Clostridium perfringens, Clostridium spiriforma and Clostridium difficile, Staphylococcus aureus, EDIM and mutants of mutant toxins such as CRM-197, non-toxic mutants of diphtheria toxin; saponins such as ISCOMs (immunostimulating complexes), chemokines, quimiokines and cytokines such as interleukins (IL-1 1L-2, IL-4, IL-5, IL-6, IL-7, IL-8, IL-12, etc), interferons (such as the interferon gamma) macrophage colony stimulating factor (M-CSF), tumor necrosis factor (TNF), defensins 1 or 2, RANTES, MIPI-alpha, and MEP-2, muramyl peptides such as N-acetyl-muramyl-L-threonyl-D-isoglutamine (thr-MDP), N-acetyl-normuramyl-L-alanyl-D-isoglutamine (nor-MDP), N-acetylmuramyl-L-alanyl-D-isoglutaminyl-L-alanine-2-(1′-2′-dipalmitoyl-s-n-glycero-3-hydroxyphosphoryloxy)-ethylamine (MTP-PE), etc.; adjuvants or immunostimulants derived from the family of CpG molecules, CpG dinucleotides and synthetic oligonucleotides which comprise CpG motifs, C. limosum exoenzyme and synthetic adjuvants such as PCPP, the cholera toxin, Salmonella toxin, alum and the like, aluminum hydroxide, N-acetyl-muramyl-L-threonyl-D-isoglutamine (thr-MDP), N-acetyl-nor-muramyl-L-alanyl-D-isoglutamine, MTP-PE and RIBI, containing three components extracted from bacteria, monophosphoryl lipid A, trehalose dimycolate and cell wall skeleton (MPL+TDM+CWS) in a squalene emulsion at 2% Tween 80. Other examples of adjuvants include DDA (dimethyl dioctadecyl ammonium bromide) and Quil-A. In a particular embodiment the adjuvant is an oily adjuvant of manide oleate and mineral oil. In another embodiment, the adjuvant is a water-in-oil emulsion. In a particular embodiment, the adjuvant is an aluminum containing adjuvant, such as aluminum hydroxide, Montanide IMS1313, diethylaminoethyl (DEAE) or a combination thereof.

The immunogenic composition or vaccine composition can be formulated or administered by any means known to one skilled in the art, such as by the parental route, such as intramuscular, subcutaneous, intradermal, transdermal or intravenous route or by the mucosal route, such as oral, ocular or intranasal route. Preferably, the immunogenic or vaccine composition is formulated for parenteral route, and more preferably for intramuscular route.

The immunogenic or vaccine composition can be formulated in unit dosage form, suitable for individual administration of precise dosages. In pulse doses, a bolus administration of the immunogenic or vaccine composition of the invention is provided, followed by a time-period wherein no composition is administered to the subject, followed by a second bolus administration. An immunologically or therapeutically effective amount of the immunogenic or vaccine composition can be administered in a single dose, or in multiple doses, for example daily, during a course of treatment. In specific, non-limiting examples, pulse doses of the immunogenic or vaccine composition are administered during the course of a day, during the course of a week, or during the course of a month. In a particular embodiment, two doses are administered, being the first dose and the second dose separated by a period of at least 1, at least 2, at least 3, at least 4, at least 5, at least 6, 7, at least 10, at least 15, at least 20, at least 21, at least 25, at least 30, at least 60, at least 90 days or more, preferably at least 21 days.

Amounts effective for therapeutic or prophylactic use can depend on the age, weight, general state of the subject, and other clinical factors. Thus, the final determination of the appropriate treatment or protocol regimen will be made by the attending clinician and/or veterinarian.

### Method for Producing the Immunogenic or Vaccine Composition and Obtained Immunogenic or Vaccine Composition

In another aspect, the invention relates to a method for producing an immunogenic or vaccine composition comprising an isolated bacterium from the species Brachyspira hyodysenteriae, hereinafter first method of the invention, wherein the method comprises the steps of:
(a) determining if the bacterium from the species Brachyspira hyodysenteriae comprises the gene on the locus BHWA1_02689 (fucT) of the B. hyodysenteriae plasmid pBHWA1 identified by the GenBank accession number NC_012226.1 or a variant of said gene of any strain of B. hyodysenteriae, or the product of said gene or variant
(b) selecting the bacterium from the species Brachyspira hyodysenteriae if said bacterium comprises the fucT gene or a variant of said gene of any strain of B. hyodysenteriae or the product of said gene or variant,
(c) inactivating the bacterium selected in step (b) and
(d) formulating the bacterium inactivated in step (c) in an immunogenic or vaccine composition.

The terms “immunogenic or vaccine composition”, “inactivated bacterium”, “Brachyspira hyodysenteriae”, “fucT” and “variant” have been previously defined in connection with the immunogenic or vaccine composition of the invention. All the particular and preferred embodiments of regarding these terms also apply to the first method of the invention.

The first step of the first method of the invention comprises determining if the bacterium from the species B. hyodysenteriae comprises the gene fucT or a variant of said gene or the product of said gene or variant.

The presence or absence of a gene can be determined by any method known by the skilled person suitable for detecting nucleic acids, for example, methods based on hybridization, such as Northern blot analysis and in situ hybridization (ISH) (including fluorescence in situ hybridization (FISH), chromogenic in situ hybridization (CISH) or silver in situ hybridization (SISH)), standard polymerase chain reaction (PCR), multiplex and/or singleplex real time RT-PCR, assays using nucleic acid arrays, Sanger sequencing, next generation sequencing (NGS), etc.

The term “gene product” as used herein, refers to both the transcriptional product of the gene (mRNA) or to the translation product of the gene (protein encoded by the gene). Therefore, the gene product can be detected by determining the presence of the mRNA of the gene or of the protein encoded by said gene.

The presence of a messenger RNA can be determined by methods well known in the art. For example, the nucleic acid contained in the blood sample is first extracted according to standard methods, for example using lytic enzymes or chemical solutions or extracted by nucleic-acid-binding resins following the manufacturer's instructions. The extracted mRNA is then detected by hybridization (e.g., Northern blot analysis or by oligonucleotide microarrays after converting the mRNA into a labelled cDNA) and/or amplification (e.g., RT-PCR). Quantitative or semi-quantitative RT-PCR is preferred. Real-time quantitative or semi-quantitative RT-PCR is particularly advantageous. Preferably, primer pairs can be designed in order to overlap an intron, to distinguish cDNA amplification from putative genomic contamination. Suitable primers may be easily designed by the skilled person. Other methods of amplification include ligase chain reaction (LCR), transcription-mediated amplification (TMA), strand displacement amplification (SDA) and nucleic acid sequence based amplification (NASBA). Preferably, the quantity of mRNA is measured by quantitative or semi-quantitative RT-PCR or by real-time quantitative or semi-quantitative RT-PCR. Other methods for determining the presence of a messenger RNA include next generation sequencing (NGS).

The presence of a protein can be determined by any method known in the art suitable for the determination and quantification of a protein in a sample. By way of a non-limiting illustration, the presence of a protein can be determined by means of a technique which comprises the use of antibodies with the capacity for binding specifically to the assayed protein (or to fragments thereof containing the antigenic determinants), or alternatively by means of a technique which does not comprise the use of antibodies such as, for example, by techniques based on mass spectroscopy. The antibodies can be monoclonal, polyclonal or fragment thereof, Fv, Fab, Fab′ and F(ab′)2, scFv, diabodies, triabodies, tetrabodies and humanized antibodies. Similarly, the antibodies may be labelled. Illustrative, but non-exclusive, examples of markers that can be herein used include radioactive isotopes, enzymes, fluorophores, chemoluminescent reagents, enzyme cofactors or substrates, enzyme inhibitors, particles, or dyes. There is a wide variety of known tests that can be used according to the present invention, such as combined application of non-labelled antibodies (primary antibodies) and labelled antibodies (secondary antibodies), Western blot or immunoblot, ELISA (enzyme-linked immunosorbent assay), RIA (radioimmunoassay), competitive EIA (enzyme immunoassay), DAS-ELISA (double antibody sandwich ELISA), two-dimensional gel electrophoresis, capillary electrophoresis, immunocytochemical and immunohistochemical techniques, immunoturbidimetry, immunofluorescence, techniques based on the use of biochips or protein microarrays including specific antibodies or assays based on the colloidal precipitation in formats such as reagent strips and assays based on antibody-linked quantum dots. Other forms of detecting proteins include, for instance, affinity chromatography techniques or ligand-binding assays.

The protein encoded by the fucT gene is an alpha-1,2-fucosyltransferase. In a particular embodiment, said protein is the alpha-1,2-fucosyltransferase from the WA1 reference strain of B. hyodysenteriae or a variant of said protein from any strain of B. hyodysenteriae. The alpha-1,2-fucosyltransferase from the WA1 reference strain of B. hyodysenteriae is the protein identified by the accession number A0A3B6VCF4 in the Uniprot (entry version 6, 17 Jun. 2020; Sequence version 1, 5 Dec. 2018). In a particular embodiment, the protein encoded by the gene fucT has the sequence of SEQ ID NO: 9. In a particular embodiment, the protein encoded by the fucT gene variant is a protein having at least 60%, at least 65%, at least 70%, at least 75%, at least 80%, at least 85%, at least 90%, at least 95%, at least 96%, at least 97%, at least 98%, at least 99% or higher sequence identity with the protein of SEQ ID NO: 9. In a particular embodiment, the protein encoded by the fucT gene variant comprises or consists of a sequence selected from the group consisting of SEQ ID NO: 83-95.

The second step of the first method of the invention comprises selecting the bacterium from the species B. hyodysenteriae if said bacterium comprises the fucT gene or a variant of said gene of any strain of B. hyodysenteriae or the product of said gene or variant.

The third step of the first method of the invention comprises inactivating the bacterium selected in step (b). The term “inactivated bacterium” has been previously defined, and methods for inactivating bacteria have been described, including for example, formaldehyde inactivation, microwave inactivation, pressure inactivation, acid inactivation, base inactivation, alcohol inactivation, peroxide inactivation, irradiation, and thermal inactivation. In a particular embodiment, the bacterium is inactivated by formaldehyde. In a particular embodiment, formaldehyde inactivation is performed by incubating the bacteria with formaldehyde at 0.01%-1.0% (v/v), for example at 0.05%-0.8% (v/v), at 0.1%-0.5% (v/v), at 0.2% (v/v). In a particular embodiment, formaldehyde inactivation is performed by incubating the bacteria with formaldehyde during 6 hours to 7 days, for example during 8 hours to 5 days, during 10 hours to 2 days, during 12 hours to 36 hours, during 24 hours. In a particular embodiment, formaldehyde inactivation is performed by incubating the bacteria with formaldehyde at 25° C. to 40° C., at 28° C. to 39° C., at 30° C. to 38° C., at 35° C. to 37° C., at 37° C. In a more particular embodiment, formaldehyde inactivation is performed by incubating the bacteria with formaldehyde at 0.2% (v/v) during 24 hours at 37° C.

The fourth step of the first method of the invention comprises formulating the inactivated bacterium or bacteria in an immunogenic or vaccine composition. The term “formulating” as used herein, means that the bacterium is mixed with one or more components suitable for administration of the bacterium to a subject, in particular to a pig. In a particular embodiment, formulating the bacterium in an immunogenic or vaccine composition comprises adding a veterinary acceptable excipient as previously defined.

In a particular embodiment, formulating the bacterium in an immunogenic or vaccine composition comprises adding an adjuvant to the bacterium. The term “adjuvant” has been previously defined in connection with the immunogenic or vaccine composition of the invention. In particular embodiment the adjuvant is an aluminum containing adjuvant, such as aluminum hydroxide, Montanide IMS1313, diethylaminoethyl (DEAE) or a combination thereof.

In a particular embodiment, the first method of the invention further comprises
determining if the bacterium is a S1 bacterium, wherein the fucT gene or the variant of said gene is comprised in a gene cluster, and wherein the gene cluster of the S1 bacterium comprises the following genes: the gene on the locus BHWA1_02695 (bcsA) of the B. hyodysenteriae plasmid pBHWA1 identified by the GenBank accession number NC_012226.1 or a variant of said gene of any strain of B. hyodysenteriae, the rfbC gene, the rfbD gene, the rfbA gene, the rfbB gene, the gene on the locus BHWA1_02690 (rgpF) of the B. hyodysenteriae plasmid pBHWA1 identified by the GenBank accession number NC_012226.1 or a variant of said gene of any strain of B. hyodysenteriae and the fucT gene or a variant of said gene of any strain of B. hyodysenteriae and/or
determining if the bacterium is a S2 bacterium, wherein the fucT gene or the variant of said gene is comprised in a gene cluster, and wherein the gene cluster of the S2 bacterium comprises the following genes: the gene on the locus BHWA1_02695 (bcsA) of the B. hyodysenteriae plasmid pBHWA1 identified by the GenBank accession number NC_012226.1 or a variant of said gene of any strain of B. hyodysenteriae, the rfbC gene, the rfbD gene, the rfbA gene, a gene having the sequence of SEQ ID NO: 8 (adoM) or a variant of said gene of any strain of B. hyodysenteriae, the rfbB gene, the gene on the locus BHWA1_02690 (rgpF) of the B. hyodysenteriae plasmid pBHWA1 identified by the GenBank accession number NC_012226.1 or a variant of said gene of any strain of B. hyodysenteriae and the fucT gene or a variant of said gene of any strain of B. hyodysenteriae.

The step of determining if a bacterium is a S1 or a S2 bacterium can be performed, as previously defined, by determining if the bacterium comprises the indicated genes of the gene cluster of S1 or S2 B. hyodysenteriae bacteria or the products of said genes.

The terms “S1 bacterium”, “S2 bacterium”, “gene cluster”, “bcsA”, “rfbC”, “rfbD”, “rfbA”, “rfbB”, “rgpF”, “AdoM”, “fucT”, and “variant” have been previously defined in connection with the immunogenic or vaccine composition of the invention. All the particular and preferred embodiments of regarding these terms also apply to the first method of the invention.

In a particular embodiment, the protein encoded by the gene bcsA is a glycosyltransferase family 2 protein encoded by the gene bcsA from the WA1 reference strain of B. hyodysenteriae or a variant of said protein from any strain of B. hyodysenteriae. In a particular embodiment, said protein is the protein identified by the accession number WP_044555473.1 in the GenBank Database (29 Apr. 2020). In a particular embodiment, the protein encoded by the gene bcsA has the sequence of SEQ ID NO: 10.

In a particular embodiment, the protein encoded by the bcsA gene variant is a protein having at least 60%, at least 65%, at least 70%, at least 75%, at least 80%, at least 85%, at least 90%, at least 95%, at least 96%, at least 97%, at least 98%, at least 99% or higher sequence identity with the protein of SEQ ID NO: 10. In a particular embodiment, the protein encoded by the bcsA gene variant comprises or consists of a sequence selected from the group consisting of SEQ ID NO: 96-99.

The protein encoded by the gene rfbA is a glucose-1-phosphate thymidylyltransferase. In a particular embodiment, said protein is the glucose-1-phosphate thymidylyltransferase from the WA1 reference strain of B. hyodysenteriae or a variant of said protein from any strain of B. hyodysenteriae. The glucose-1-phosphate thymidylyltransferase from the WA1 reference strain of B. hyodysenteriae is the protein identified by the accession number A0A3B6VFJ0_BRAHW in the Uniprot (entry version 5, 11 Dec. 2019; Sequence version 1, 5 Dec. 2018). In a particular embodiment, the protein encoded by the gene rfbA has the sequence of SEQ ID NO: 11.

In a particular embodiment, the protein encoded by the rfbA gene variant is a protein having at least 60%, at least 65%, at least 70%, at least 75%, at least 80%, at least 85%, at least 90%, at least 95%, at least 96%, at least 97%, at least 98%, at least 99% or higher sequence identity with the protein of SEQ ID NO: 11. In a particular embodiment, the protein encoded by the rfbA gene variant comprises or consists of a sequence selected from the group consisting of SEQ ID NO: 100-104.

The protein encoded by the gene rfbB is a dTDP-glucose 4,6-dehydratase. In a particular embodiment, said protein is the dTDP-glucose 4,6-dehydratase from the WA1 reference strain of B. hyodysenteriae or a variant of said protein from any strain of B. hyodysenteriae. The dTDP-glucose 4,6-dehydratase from the WA1 reference strain of B. hyodysenteriae is the protein identified by the accession number A0A3B6VCL3_BRAHW in the Uniprot (entry version 6, 11 Dec. 2019; Sequence version 1, 5 Dec. 2018). In a particular embodiment, the protein encoded by the gene rfbB has the sequence of SEQ ID NO: 12.

In a particular embodiment, the protein encoded by the rfbB gene variant is a protein having at least 60%, at least 65%, at least 70%, at least 75%, at least 80%, at least 85%, at least 90%, at least 95%, at least 96%, at least 97%, at least 98%, at least 99% or higher sequence identity with the protein of SEQ ID NO: 12. In a particular embodiment, the protein encoded by the rfbB gene comprises or consists of a sequence selected from the group consisting of SEQ ID NO: 105-108.

The protein encoded by the gene rfbC is a dTDP-4-dehydrorhamnose 3,5-epimerase. In a particular embodiment, said protein is the dTDP-4-dehydrorhamnose 3,5-epimerase from the WA1 reference strain of B. hyodysenteriae or a variant of said protein from any strain of B. hyodysenteriae. The dTDP-4-dehydrorhamnose 3,5-epimerase from the WA1 reference strain of B. hyodysenteriae is the protein identified by the accession number A0A3B6VCK2_BRAHW in the Uniprot (entry version 8, 17 Jun. 2020; Sequence version 1, 5 Dec. 2018). In a particular embodiment, the protein encoded by the gene rfbC has the sequence of SEQ ID NO: 13.

In a particular embodiment, the protein encoded by the rfbC gene variant is a protein having at least 60%, at least 65%, at least 70%, at least 75%, at least 80%, at least 85%, at least 90%, at least 95%, at least 96%, at least 97%, at least 98%, at least 99% or higher sequence identity with the protein of SEQ ID NO: 13. In a particular embodiment, the protein encoded by the rfbC gene comprises or consists of a sequence selected from the group consisting of SEQ ID NO: 109-111.

The protein encoded by the gene rfbD is a dTDP-4-dehydrorhamnose reductase. In a particular embodiment, said protein is the dTDP-4-dehydrorhamnose reductase from the WA1 reference strain of B. hyodysenteriae or a variant of said protein from any strain of B. hyodysenteriae. The dTDP-4-dehydrorhamnose reductase from the WA1 reference strain of B. hyodysenteriae is the protein identified by the accession number A0A3B6VIP7_BRAHW in the Uniprot (entry version 5, 11 Dec. 2019; Sequence version 1, 5 Dec. 2018). In a particular embodiment, the protein encoded by the gene rfbD has the sequence of SEQ ID NO: 14.

In a particular embodiment, the protein encoded by the rfbD gene variant is a protein having at least 60%, at least 65%, at least 70%, at least 75%, at least 80%, at least 85%, at least 90%, at least 95%, at least 96%, at least 97%, at least 98%, at least 99% or higher sequence identity with the protein of SEQ ID NO: 14. In a particular embodiment, the protein encoded by the rfbD gene comprises or consists of a sequence selected from the group consisting of SEQ ID NO: 112-117.

The protein encoded by the gene rgpF is a lipopolysaccharide biosynthesis protein-like protein. In a particular embodiment, said protein is the lipopolysaccharide biosynthesis protein-like protein from the WA1 reference strain of B. hyodysenteriae or a variant of said protein from any strain of B. hyodysenteriae. The lipopolysaccharide biosynthesis protein-like protein from the WA1 reference strain of B. hyodysenteriae is the protein identified by the accession number 0A3B6VGX0_BRAHW in the Uniprot (entry version 5, 11 Dec. 2019; Sequence version 1, 5 Dec. 2018). In a particular embodiment, the protein encoded by the gene rgpF has the sequence of SEQ ID NO: 15.

In a particular embodiment, the protein encoded by the rgpF gene variant is a protein having at least 60%, at least 65%, at least 70%, at least 75%, at least 80%, at least 85%, at least 90%, at least 95%, at least 96%, at least 97%, at least 98%, at least 99% or higher sequence identity with the protein of SEQ ID NO: 15. In a particular embodiment, the protein encoded by the rgpF gene comprises or consists of a sequence selected from the group consisting of SEQ ID NO: 118-123.

The protein encoded by the gene adoM is a methyltransferase domain-containing protein. In a particular embodiment, the protein encoded by the gene adoM has the sequence of SEQ ID NO: 16 or a variant of said protein from any strain of B. hyodysenteriae.

In a particular embodiment, the protein encoded by the adoM variant is a protein having at least 60%, at least 65%, at least 70%, at least 75%, at least 80%, at least 85%, at least 90%, at least 95%, at least 96%, at least 97%, at least 98%, at least 99% or higher sequence identity with the protein of SEQ ID NO: 16.

In a more particular embodiment, the first method of the invention further comprises
determining if the gene cluster of the S1 bacterium comprises, from the 5′ to the 3′ end, the following genes: bcsA or a variant of said gene of any strain of B. hyodysenteriae, rfbC, rfbD, rfbA, rfbB, rgpF or a variant of said gene of any strain of B. hyodysenteriae and fucT or a variant of said gene of any strain of B. hyodysenteriae;
and/or
determining if the gene cluster of the S2 bacterium comprises, from the 5′ to the 3′ end, the following genes: bcsA or a variant of said gene of any strain of B. hyodysenteriae, rfbC, rfbD, rfbA, adoM or a variant of said gene of any strain of B. hyodysenteriae, rfbB, rgpF or a variant of said gene of any strain of B. hyodysenteriae and fucT or a variant of said gene of any strain of B. hyodysenteriae.

In a particular embodiment, at least two strains of a S1 bacterium are selected. In a particular embodiment, the two strains are selected from the group consisting of B-7493, B-7500, B-8295 and B-7482. In a more particular embodiment, the two strains are B-7500 and B-8295.

In another particular embodiment, at least two strains of a S2 bacterium are selected. In a particular embodiment, the two strains are selected from the group consisting of B-8397, B-7484, and B-7494. In a more particular embodiment, the two strains are B-8397 and B-7484.

In another particular embodiment, at least two strains of a S1 bacterium and at least one strain of a S2 bacterium are selected. In a more particular embodiment, S1 strains B-7500 and B-8295 and S2 strain B-8397 are selected.

In another particular embodiment, at least one strain of a S1 bacterium and at least two strains of a S2 bacterium are selected.

In another particular embodiment, at least two strains of a S1 bacterium and at least two strains of a S2 bacterium are selected. In a more particular embodiment, S1 strains B-7500 and B-8295 and S2 strains B-8397 and B-7484 are selected.

In another particular embodiment, at least three strains of a S1 bacterium and at least two strains of a S2 bacterium are selected. In a more particular embodiment, S1 strains B-7500, B-8295 and B-7482 and S2 strains B-8397 and B-7484 are selected.

In another aspect, the invention relates to an immunogenic or vaccine composition obtainable by, obtained by or directly obtained by the first method of the invention. In a particular embodiment, the immunogenic or vaccine composition obtained by the first method of the invention is the immunogenic or vaccine composition previously defined in the first aspect of the invention.

Methods for Selecting a Bacterium from the Species B. hyodysenteriae

As shown in Examples 2 and 3, vaccines against swine dysentery comprising inactivated S1 or S2 B. hyodysenteriae bacteria are capable of reducing the clinical signs of the disease. Both types of bacteria comprise a gene cluster comprising the fucT gene, which is absent in S0 and S3 bacteria. Therefore, the presence or absence of fucT gene or of its gene product could be used for selecting a bacterium useful for producing a vaccine against swine dysentery.

Therefore, in another aspect, the invention relates to a method for selecting an isolated bacterium from the species Brachyspira hyodysenteriae useful for manufacturing a vaccine against swine dysentery, hereinafter second method of the invention, comprising determining the presence or absence of the gene on the locus BHWA1_02689 (fucT) of the B. hyodysenteriae plasmid pBHWA1 identified by the GenBank accession number NC_012226.1 or a variant of said gene of any strain of B. hyodysenteriae and/or of the product of said gene or variant, wherein if the bacterium comprises said gene or variant or the product of said gene or variant, the bacterium is selected for manufacturing a vaccine against swine dysentery.

The terms “vaccine”, “Brachyspira hyodysenteriae”, “fucT” and “variant” have been previously defined in connection with the immunogenic or vaccine composition of the invention. All the particular and preferred embodiments of the first aspect of the invention regarding these terms also apply to the second method of the invention.

The term “swine dysentery”, as used herein, refers to an infectious disease caused by Brachyspira hyodysenteriae and that affects swine, characterized by mucohemorrhagic diarrhea and marked inflammation limited to the large intestine (cecum and/or colon). All ages of swine may have SD although it seldom is apparent in piglets less than three weeks old. The disease occurs more frequently during the growing/finishing periods. As used herein the term “disease caused by Brachyspira hyodysenteriae” in particular relates to dysentery caused by B. hyodysenteriae, more particular to swine dysentery. The term “clinical signs caused by Brachyspira hyodysenteriae” in particular relates to any clinical sign selected from mucus and/or blood in feces (dysentery), diarrhoea, weight loss, lesions in the large intestine, spirochaete (Brachyspira hyodysenteriae) in the large intestine.

The second method of the invention comprises determining the presence or absence of the fucT gene or of a variant of said gene of any strain of B. hyodysenteriae or of the product of said gene or variant. The presence or absence of the fucT gene can be determined by any suitable method for detecting the presence of nucleic acids previously described.

The term “gene product” has been previously defined.

In a particular embodiment of the second method of the invention, the bacterium from the species B. hyodysenteriae is a wild-type bacterium, that is, a bacterium having a genome that has not been artificially modified.

In a particular embodiment, the second method of the invention further comprises:
(a) determining if the bacterium from the species Brachyspira hyodysenteriae comprises the following genes or the products of the following genes: the gene on the locus BHWA1_02695 (bcsA) of the B. hyodysenteriae plasmid pBHWA1 identified by the GenBank accession number NC_012226.1 or a variant of said gene of any strain of B. hyodysenteriae, the rfbC gene, the rfbD gene, the rfbA gene, the rfbB gene, the gene on the locus BHWA1_02690 (rgpF) of the B. hyodysenteriae plasmid pBHWA1 identified by the GenBank accession number NC_012226.1 or a variant of said gene of any strain of B. hyodysenteriae and the fucT gene or a variant of said gene of any strain of B. hyodysenteriae,
wherein if the bacterium comprises said genes or the products of said genes the bacterium is selected for manufacturing a vaccine against swine dysentery,
and/or
(b) determining if the bacterium from the species B. hyodysenteriae comprises the following genes or the products of the following genes: the gene on the locus BHWA1_02695 (bcsA) of the B. hyodysenteriae plasmid pBHWA1 identified by the GenBank accession number NC_012226.1 or a variant of said gene of any strain of B. hyodysenteriae, the rfbC gene, the rfbD gene, the rfbA gene, a gene having the sequence of SEQ ID NO: 8 (adoM) or a variant of said gene of any strain of B. hyodysenteriae, the rfbB gene, the gene on the locus BHWA1_02690 (rgpF) of the B. hyodysenteriae plasmid pBHWA1 identified by the GenBank accession number NC_012226.1 or a variant of said gene of any strain of B. hyodysenteriae and the fucT gene or a variant of said gene of any strain of B. hyodysenteriae,
wherein if the bacterium comprises said genes or the products of said genes the bacterium is selected for manufacturing a vaccine against swine dysentery.

The term “gene product” has been previously defined, as well as methods for determining the presence or absence of the product of a gene.

### Medical Uses

In another aspect, the invention relates to the immunogenic or vaccine composition of the invention or to the immunogenic or vaccine composition obtained by the first method of the invention for use as a medicament.

Alternatively, the invention relates to the use of the immunogenic or vaccine composition of the invention or of the immunogenic or vaccine composition obtained by the first method of the invention for the manufacture of a medicament.

In another aspect, the invention relates to the immunogenic composition of the invention or to the immunogenic composition obtained by the first method of the invention for use in inducing an immune response against a bacterium from the species Brachyspira hyodysenteriae.

Alternatively, the invention relates to use of the immunogenic composition of the invention or of the immunogenic composition obtained by the first method of the invention for the manufacture of a medicament for inducing an immune response against a bacterium from the species Brachyspira hyodysenteriae.

Alternatively, the invention relates to a method for inducing an immune response against a bacterium from the species Brachyspira hyodysenteriae comprising administering to a subject an immunologically or therapeutically effective amount of the immunogenic composition of the invention or of the immunogenic composition obtained by the first method of the invention.

In another aspect, the invention relates to the vaccine composition of the invention or to the vaccine composition obtained by the first method of the invention for use in the protection against an infection caused by a bacterium from the species Brachyspira hyodysenteriae.

Alternatively, the invention relates to the use of the vaccine composition of the invention or of the vaccine composition obtained by the first method of the invention for the manufacture of a medicament for the protection against an infection caused by a bacterium from the species Brachyspira hyodysenteriae.

Alternatively, the invention relates to a method for preventing an infection caused by a bacterium from the species Brachyspira hyodysenteriae in a subject comprising administering to a subject an immunologically or therapeutically effective amount of the vaccine composition of the invention or of the vaccine composition obtained by the first method of the invention.

In another aspect, the invention relates to the vaccine composition of the invention or the vaccine composition obtained by the first method of the invention for use in the treatment and/or prevention of swine dysentery.

Alternatively, the invention relates to the use of the vaccine composition of the invention or of the vaccine composition obtained by the first method of the invention for the manufacture of a medicament for the prevention and/or treatment of swine dysentery.

Alternatively, the invention relates to a method for treating and/or preventing swine dysentery comprising administering to a subject an immunologically or therapeutically effective amount of vaccine composition of the invention or of the vaccine composition obtained by the first method of the invention.

In a particular embodiment of the medical uses of the invention, the vaccine composition of the invention or the vaccine composition obtained by the first method of the invention induces a reduction in the clinical signs of the infection by a bacterium from the species Brachyspira hyodysenteriae. In a more particular embodiment, said clinical signs are diarrhea score, intestinal lesion score and/or average daily weight loss. The term “reduction of the clinical signs of a disease caused by Brachyspira hyodysenteriae” or “reduction of symptoms associated with Brachyspira hyodysenteriae infection”, respectively, means, but is not limited to, reducing the number of infected subjects in a group, reducing or eliminating the number of subjects exhibiting clinical symptoms of infection, or reducing the severity of any clinical symptoms that are present in the subjects, in comparison to wild-type infection. For example, it should refer to any reduction of pathogen load, pathogen shedding, reduction in pathogen transmission, or reduction of any clinical sign of Brachyspira hyodysenteriae infection, in particular selected from mucus and/or blood in feces (dysentery), diarrhea, weight loss, lesions in the large intestine, spirochaete (Brachyspira hyodysenteriae) in the large intestine. Preferably, these clinical signs are reduced in subjects receiving the immunogenic composition of the invention by at least 10% in comparison to subjects not receiving the composition and may become infected. More preferably, clinical signs are reduced in subjects receiving the composition of the present invention by at least 20%, preferably by at least 30%, preferably by at least 40%, preferably by at least 50%, preferably by at least 60%, preferably by at least 70%, preferably by at least 80%, preferably by at least 90%, preferably by at least 95%, preferably by at least 99% or even more preferably by at least 100% of the clinical signs, particularly, diarrhea score, intestinal lesion score and average daily weight loss in comparison to subjects not receiving the composition of the invention.

In a particular embodiment of the medical uses of the invention, the vaccine composition of the invention or the vaccine composition obtained by the first method of the invention prevents or delays the onset of the clinical signs of the infection by a bacterium from the species Brachyspira hyodysenteriae.

In a particular embodiment of the medical uses of the invention, the vaccine composition of the invention or the vaccine composition obtained by the first method of the invention induces an increase in the survival rate in subjects suffering from an infection by a bacterium from the species Brachyspira hyodysenteriae. The term “survival rate” and “increased survival rate” has been previously defined.

The term “subject” or “host”, as used herein, refers to the target individuals in need thereof to whom the immunogenic composition or vaccine of the invention are administered, among others humans, mammals, livestock, or any other animal species susceptible to be vaccinated with the composition of the invention. Preferably, the mammal is a porcine specie, more preferably is swine or pig. As used herein, the term “pig” or “swine” is intended for porcine species including, among others, pigs, boars, sows, gilts and piglets of any age or in any phase of their production cycle; it is also intended for sows and gilts, and more particularly for piglets. A gilt is a female pig approximately under the age of 1 year. The term refers to a pig who has not farrowed or given birth to a litter or progeny. Once a pig has had a litter or progeny and is past approximately her first year, the pig is known as sow.

The term “treatment”, as used herein, refers to any type of therapy, which is aimed at terminating, preventing, ameliorating or reducing the susceptibility to a clinical condition or existing disease as described herein, including complete curing of a disease as well as amelioration or alleviation of said disease. In a preferred embodiment, the term treatment relates to prophylactic treatment (i.e. a therapy to reduce the susceptibility to a clinical condition), of a disorder or a condition as defined herein. Thus, “treatment,” “treating,” and their equivalent terms refer to obtaining a desired pharmacologic or physiologic effect, covering any treatment of a pathological condition or disorder in a mammal, including a swine. The effect may be prophylactic in terms of completely or partially preventing a disorder or symptom thereof and/or may be therapeutic in terms of a partial or complete cure for a disorder and/or adverse effect attributable to the disorder. That is, “treatment” includes (1) preventing the disorder from occurring or recurring in a subject, (2) inhibiting the disorder, such as arresting its development, (3) stopping or terminating the disorder or, at least, clinical signs associated therewith, so that the host no longer suffers from the disorder or its clinical signs, such as causing regression of the disorder or its clinical signs, for example, by restoring or repairing a lost, missing or defective function, or stimulating an inefficient process, or (4) relieving, alleviating, or ameliorating the disorder, or clinical signs associated therewith, where ameliorating is used in a broad sense to refer to at least a reduction in the magnitude of a parameter, such as inflammation, pain, immune deficiency, reduction of the pathogen load, the pathogen shedding, or reduction in pathogen transmission.

The term “prevention” or “reduction” or “preventing” or “reducing”, respectively, as used herein, means, but is not limited to a process of prophylaxis in which a subject is exposed to the immunogenic or vaccine composition of the invention prior to the induction or onset of the disease process, and wherein said immunogenic or vaccine composition, when administered to said pig, elicits or is able to elicit an immune response in said pig against B. hyodysenteriae. Altogether, such treatment results in reduction of the clinical signs of swine dysentery or of clinical signs associated with swine dysentery, respectively or reduction of bacteria titer. More specifically, the term “prevention” or “preventing”, as used herein, means generally a process of prophylaxis in which a pig is exposed to the immunogenic or vaccine composition of the present invention prior to the induction or onset of swine dysentery.

Herein, “reduction of clinical signs associated with swine dysentery” means, but is not limited to, reducing the number of infected subjects in a group, reducing or eliminating the number of subjects exhibiting clinical signs of infection, or reducing the severity of any clinical signs that are present in the subjects, in comparison to wild-type infection. For example, it should refer to any reduction of bacteria titer, reduction in bacteria transmission, or reduction of any clinical sign of swine dysentery. Preferably these clinical signs are reduced in subjects receiving the composition of the present invention by at least 10 percent in comparison to subjects not receiving the composition and may become infected. More preferably, clinical signs are reduced in subjects receiving the composition of the present invention by at least 20 percent, preferably by at least 30 percent, more preferably by at least 40 percent, and even more preferably by at least 50 percent in comparison to subjects not receiving the composition and may become infected. The clinical signs, as mentioned herein, can be selected from the group consisting of diarrhea, intestinal lesion and bacteria titer in the colon.

In a particular embodiment, the administration of the immunogenic or vaccine composition of the invention or of the immunogenic or vaccine composition obtained by the method of the invention is able to reduce and/or prevent the clinical signs of swine dysentery when administered to pigs that are subsequently infected with B. hyodysenteriae when compared to pigs that do not receive the immunogenic or vaccine composition of the invention or the immunogenic or vaccine composition obtained by the method of the invention. Particularly, said immunogenic or vaccine compositions reduce the diarrhea score, intestinal lesion score and daily weight loss and increases the survival rate in the vaccinated pigs infected with B. hyodysenteriae. In a more particular embodiment, the administration of said immunogenic or vaccine composition induces at least 10%, at least 20%, at least 30%, at least 40%, at least 50%, at least 60%, at least 70%, at least 80%, at least 90%, at least 95%, at least 98%, at least 99%, at least 100% or higher reduction in the clinical signs of swine dysentery, particularly diarrhea score, intestinal lesion and daily weight loss, and induces an increase in the survival rate of at least 10%, at least 20%, at least 30%, at least 40%, at least 50%, at least 60%, at least 70%, at least 80%, at least 90%, at least 95%, at least 98%, at least 99%, at least 100% or higher when administered to pigs that are subsequently infected with B. hyodysenteriae when compared to pigs that do not receive the immunogenic or vaccine composition of the invention or the immunogenic or vaccine composition obtained by the method of the invention.

The terms “diarrhea score”, “intestinal lesion score”, “daily weight loss” and “survival rate” have been previously defined.

The term “immunologically effective amount” or “therapeutically effective amount”, as used herein, relates to the sufficient amount of the composition of the invention to provide the desired effect, i.e. to achieve an appreciable prevention, cure, delay, reduction of severity or amelioration of one or more clinical signs derived from the disease, and will generally be determined by, among other causes, the characteristics of the agent itself (the B. hyodysenteriae bacteria) and the therapeutic effect to be achieved. As used herein “immunologically effective amount” or “therapeutically effective amount” also means an amount sufficient to reduce any clinical signs as previously defined by at least 10%, preferably by at least 20%, preferably by at least 30%, preferably by at least 40%, preferably by at least 50%, preferably by at least 60%, preferably by at least 70%, preferably by at least 80%, preferably by at least 90%, preferably by at least 95%, preferably by at least 99%, and more preferably by at least 100% compared to a non-vaccinated control group. It will also depend on the subject to be treated, the severity of the disease suffered by said subject, the chosen dosage form, etc. For this reason, the doses that may be mentioned in this invention must be considered only as guides for the person skilled in the art, who must adjust the doses depending on the aforementioned variables. In an embodiment, the effective amount produces the amelioration of one or more clinical signs of the disease that is being treated. Even though individual needs vary, determination of optimal ranges for immunologically or therapeutically effective amounts of the compounds according to the invention belongs to the common experience of those experts in the art. In general, the dosage needed to provide an effective treatment or prevention, which can be adjusted by one expert in the art, will vary depending on age, health, fitness, sex, diet, weight, degree of alteration of the receptor, frequency of treatment, nature and condition of the injury, nature and extent of impairment or illness, medical condition of the subject, route of administration, pharmacological considerations such as activity, efficacy, pharmacokinetic and toxicology profile of the particular compound used, if using a system drug delivery, and if the compound is administered as part of a combination of drugs.

In a particular embodiment, the immunogenic or vaccine composition comprises or consists of at least two strains of a S1 bacterium. In a particular embodiment, the two strains are selected from the group consisting of B-7493, B-7500, B-8295 and B-7482. In a more particular embodiment, the two strains are B-7500 and B-8295.

In another particular embodiment, the immunogenic or vaccine composition comprises at least two strains of a S2 bacterium. In a particular embodiment, the two strains are selected from the group consisting of B-8397, B-7484, and B-7494. In a more particular embodiment, the two strains are B-8397 and B-7484.

In another particular embodiment, the immunogenic or vaccine composition comprises at least two strains of a S1 bacterium and at least one strain of a S2 bacterium. In a more particular embodiment, the immunogenic or vaccine composition comprises or consists of S1 strains B-7500 and B-8295 and S2 strain B-8397.

In another particular embodiment, the immunogenic or vaccine composition comprises at least one strain of a S1 bacterium and at least two strains of a S2 bacterium.

In another particular embodiment, the immunogenic or vaccine composition comprises or consists of at least two strains of a S1 bacterium and at least two strains of a S2 bacterium. In a more particular embodiment, the immunogenic or vaccine composition comprises or consists of the S1 strains B-7500 and B-8295 and the S2 strains B-8397 and B-7484.

In another particular embodiment, the immunogenic or vaccine composition comprises or consists of at least three strains of a S1 bacterium and at least two strains of a S2 bacterium. In a more particular embodiment, the immunogenic or vaccine composition comprises or consists of the S1 strains B-7500, B-8295 and B-7482 and the S2 strains B-8397 and B-7484.

The term “cross-protection” implies terminating, preventing, ameliorating or reducing the susceptibility of a B. hyodysenteriae infections or clinical signs of swine dysentery caused by at least one B. hyodysenteriae strain that is different from the strains that are present in the immunogenic or vaccine composition.

The term “heterologous strain”, as used herein, refers to a strain that is different from the strains that are comprised in the immunogenic or vaccine composition.

In a particular embodiment, the immunogenic composition induces an immune response against a strain of B. hyodysenteriae different from the strain that is comprised in the immunogenic composition or, alternatively the immunogenic composition is able to confer cross-protection against a strain of B. hyodysenteriae different from the strain of B. hyodysenteriae that is comprised in the immunogenic composition.

In a particular embodiment, the vaccine composition induces protection against a strain of B. hyodysenteriae different from the strain comprised in the vaccine or, alternatively the vaccine composition induces cross-protection against a strain of B. hyodysenteriae different from the strain of B. hyodysenteriae that is comprised in the vaccine composition.

In a particular embodiment of the medical uses of the invention, the vaccine composition of the invention or the vaccine composition obtained by the first method of the invention induces a reduction in the clinical signs of the infection by a strain of bacterium from the species Brachyspira hyodysenteriae different from the strain that is comprised in the vaccine composition when compared to subjects that do not receive the vaccine composition. In a more particular embodiment, said clinical signs are diarrhea score, intestinal lesion score and/or average daily weight loss. In a particular embodiment, the reduction is of at least 5%, at least 10%, at least 20%, at least 30%, at least 40%, at least 50%, at least 60%, at least 70%, at least 80%, at least 90%, at least 95% or 100% of the clinical signs, particularly, diarrhea score, intestinal lesion score and average daily weight loss, when compared to subjects that do not receive the vaccine composition of the invention.

In a particular embodiment of the medical uses of the invention, the vaccine composition of the invention or the vaccine composition obtained by the first method of the invention prevents or delays the onset of the clinical signs of the infection by a strain of bacterium from the species Brachyspira hyodysenteriae different from the strain comprised in the vaccine composition.

In a particular embodiment of the medical uses of the invention, the vaccine composition of the invention or the vaccine composition obtained by the first method of the invention induces an increase in the survival rate in subjects suffering from an infection by a strain of a bacterium from the species Brachyspira hyodysenteriae different from the strain comprised in the vaccine composition. In a particular embodiment, the vaccine composition of the invention or the vaccine composition obtained by the first method of the invention induces an increase in the survival rate after 1 to 200 days post-vaccination, for example after 2 to 190 days post-vaccination, after 3 to 180 days post-vaccination, after 4 to 170 days post-vaccination, after 5 to 150 days post-vaccination, after 6 to 130 days post-vaccination, after 7 to 100 days post-vaccination, after 10 to 75 days post-vaccination, after 12 to 50 days post-vaccination, after 20 days post-vaccination in subjects suffering from an infection by a bacterium from the species Brachyspira hyodysenteriae. The term “increased in the survival rate” refers to any increased compared with non-vaccinated control subjects, for example, an increase of at least 5%, at least 10%, at least 20%, at least 30%, at least 40%, at least 50%, at least 60%, at least 70%, at least 80%, at least 90%, at least 95%, at least 100% or higher.

In a particular embodiment, the immunogenic or vaccine composition is administered by intramuscular, subcutaneous, intradermal, transdermal, intravenous, oral, ocular or intranasal route. In a particular embodiment, the immunogenic or vaccine composition is administered by intramuscular, subcutaneous, intradermal, transdermal or intravenous route. In a particular embodiment, the immunogenic or vaccine composition is administered by intramuscular route.

In a particular embodiment, the immunogenic or vaccine composition comprises an immunologically or therapeutically effective amount of an isolated and inactivated B. hyodysenteriae strain.

In a particular embodiment, the immunogenic or vaccine composition is administered at a dose of at least 10⁸, at least 10⁹, at least 10¹⁰, at last 10¹¹ bacteria of each strain/dose.

In a particular embodiment, two or more doses of the immunogenic or vaccine composition are administered, preferably two doses, being the doses separated by least 1, at least 2, at least 3, at least 4, at least 5, at least 6, at least 7, at least 10, at least 14, at least 20, at least 21, at least 25, at least 30, at least 60, at least 90 days or more, preferably at least 14 or at least 21 days.

### EXAMPLES

### Example 1: Determination of the Genomic Structure of Brachyspira hyodysenteriae Isolates Based on the 36 kb Plasmid (pBH 36 kb)

The objective of this study was to analyze Brachyspira hyodysenteriae (Bhyo) isolates from different geographical origin by determining the architecture of a gene cluster comprising the genes of the operon rfbAD (also known as rfbBADC) located at the plasmid pBH of 36 kb in reference to the standard WA1 Brachyspira hyodysenteriae isolate.

The genes rfbA, rfbB, rfbC, rfbD are located forming an operon in the plasmid pBH of 36 kb in the B. hyodysenteriae strain WA1 (GenBank No. NC_012226.1). This operon rfbBADC groups the genes related to the lipooligosaccharide synthesis, grouping 4 of the 6 genes rfb present in the plasmid.

### Materials and Methods

49 isolated Bhyo strains from different geographical origins isolated from pigs with confirmed clinical cases of swine dysentery caused by B. hyodysenteriae were used in this study. The 49 strains included isolates from Argentina (3), Australia (3), Belgium (4), Canada (1), Germany (4), Japan (5), Portugal (1), Spain (17), Sweden (5), United Kingdom (1), and USA (5).

The DNA of the different isolates was obtained by SDS lysis. B. hyodysenteriae strain cultures of 15 ml were centrifuged at 4000 g for 10 minutes and resuspended in 0.5 ml of 10 mM Tris pH 8, 0.5M NaCl and 10 mM EDTA. Subsequently, 10 μL of 20% SDS were added and the lysate was deproteinized twice with a volume of Phenol-Chloroform. The DNA was then precipitated by adding two volumes of absolute ethanol. DNA was recovered both by simple decantation and by sedimentation at 13000 g for 5 minutes. Once washed with 70% ethanol and dried, the DNAs were finally dissolved in 50-100 μL of 10 mM Tris and 1 mM EDTA.

The DNA samples obtained were used as starting material to perform ultrasequencing in an Illumina Myseq.

For mass sequencing (Shotgun) and direct from the extracted DNAs, the Nextera kit (Paired ends, Illumina) was used, which produces fragments of 400-600 bases. During sequencing, an Illumina MySeq apparatus with a 2×150 base reading chip was used, which determines the sequence of this number of bases for each end of said fragments. In typical sequencing, the sequence of 1-4 million fragments is determined. The first analysis of the sequences obtained was performed by aligning them with the reference sequence of the 36 kb plasmid pBH of the WA1 reference strain. For this, the Bowtie2 (Langmead et al., Nature Methods 2012, 357-359), SamTools (Li et al., Bioinformatics, 2008, 2078-2079) and VarScan (Koboldt et al., Genome Research, 2012, 568-576) programs were used. De novo assembly was performed on the sequences of the isolates that had said plasmid to establish the arrangement of genes in the plasmid skeleton. Assembling “de novo” of the fragments obtained is not a trivial task. There are several bioinformatic packages, and most of them use algorithms based on graph theory (Bruijin's graphs). In this case, the genomes of the different isolates have been assembled using the Velvet (Zerbino et al., Genome Research, 2009, 821-829) and SPAdes (Bankevich et al., Journal of Computational biology, 2012, 455-477) programs. The identification of ORFs on the obtained contigs was performed using the Glimmer program (NCBI). Multiple alignments of the polypeptide sequences were performed using the ClustalX v2.1 program.

Once the sequences for all the isolated Bhyo strains were obtained, the presence or absence of the 36 kb plasmid pBH in all sequenced isolates was studied. This was done by aligning all the sequences obtained with that of the cdsM operon, which contains the replication and metabolism genes of the plasmid. The presence of the cdsM operon was used as the criteria for determining the presence of the pBH plasmid in each isolate. After assessing the presence or absence of the 36 kb pBH plasmid in the different Bhyo isolates, the gene distribution of the cluster containing the operon rfbAD was studied for each strain. To do so, the 36 kb pBH plasmid sequences were aligned with those of the rfbAD operon and the adjacent genes of the 36 kb pBH plasmid of the Bhyo reference strain WA1.

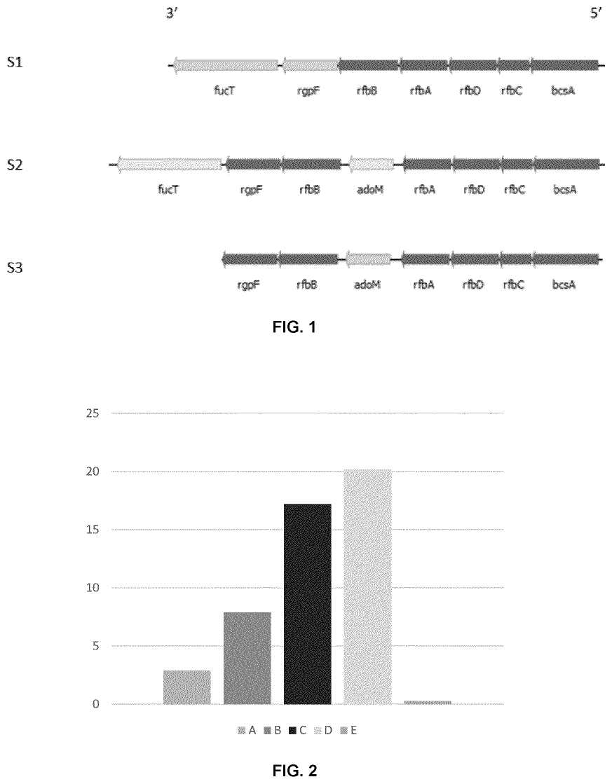

Once the presence of the rfbAD genes was confirmed, the architecture of the gene cluster containing said operon together with the adjacent genes was determined in all the isolates carrying the 36 kb plasmid pBH. For this, a “de novo” assembly was performed with the results of the ultrasequencing and annotated to identify the presence or the absence of the different genes of this operon and the adjacent genes. After the annotation, the architecture of the cluster for all the samples analyzed was determined. The analysis of the gene distribution surprisingly suggested the existence of three different structures of the gene cluster containing the operon rfbAD of the 36 kb plasmid pBH among the isolates (FIG. 1). A first gene cluster structure, designated as structure 1 (S1), includes the isolates that have an identical structure to the plasmid 36 kb pBH of the reference Bhyo WA1 strain. A second gene cluster structure, designated as structure 2 (S2), includes isolates that have the insertion of the adoM gene between the genes rfbA and rfbB. A third gene cluster structure, designated as structure 3 (S3), incudes isolates that have the insertion of the adoM gene between the genes rfbA and rfbB but lack the gene fucT (homologous to the gene BHWA1_02689 characterized in the 36 kb plasmid pBH of reference strain WA1, GenBank No. NC_012226.1), which codes for an α-1,2-fucosyltransferase. It was also found that some Bhyo isolates did not have the 36 kb plasmid pBH, thus not comprising the identified genetic cluster as the other groups (S1, S2 and S3).

The following table shows the allocation of the different Bhyo strains based on the structure of the gene cluster comprising the rfbAD operon.

TABLE 1
Distribution of the 49 Bhyo isolates analyzed by the genomic
structure of the gene cluster comprising the rfbAD operon.

| Family | No. Isolates | Cluster of genes in pBH of 36 kb (5′ to 3′) |
|---|---|---|
| S1 | 27 | bcsA, rfbC, rfbD, rfbA, rfbB, rgpF, fucT |
| S2 | 4 | bcsA, rfbC, rfbD, rfbA, adoM, rfbB, rgpF, |
| | | fucT |
| S3 | 11 | bcsA, rfbC, rfbD, rfbA, adoM, rfbB, rfpF |
| S0 | 7 | No plasmid of 36 kb |

This study confirms the presence of a 36 kb pBH plasmid in most isolates of Bhyo obtained from different geographical origins and coming from clinical cases of swine dysentery. A different genomic structure based on the gene cluster comprising the rfbAD operon was found for the Bhyo isolates allowing its classification in four groups (S1 to S3 and S0).

### Example 2: Efficacy Study of Different Monovalent Experimental Vaccines Against an Experimental Infection of B. hyodysenteriae Strain B-7493

A total of 50 pigs between 5 and 6 weeks of age were chosen for this study. The animals were randomly assigned into 5 groups of 10 pigs each one (Groups A to E). On Day 0 pigs received a first dose of an experimental vaccine according to the group assignment. Three weeks later (Day 21) pigs received a second dose of the vaccine. Vaccines were administered at the neck by intramuscular route at 2 mL for each administration.
Group A received a vaccine comprising 3.33×10⁹ total bacteria (BT) per dose of the inactivated B. hyodysenteriae strain B-7493, USA isolate. This USA isolate showed a genomic arrangement consistent with the gene cluster structure S1 (as determined in Example 1). This vaccine was formulated with the same strain as the one used for the experimental infection, thus it is a homologous vaccine. The vaccine was also formulated at 50% v/v with a commercial adjuvant, Montanide IMS 1313.
Group B received a vaccine comprising 3.33×10⁹ total bacteria (BT) per dose of the inactivated B. hyodysenteriae strain B-7500, Spanish isolate. This Spanish isolate showed a genomic arrangement consistent with the gene cluster structure S1 (as determined in Example 1). The vaccine was formulated at 50% v/v with the commercial adjuvant Montanide IMS 1313.
Group C received a vaccine comprising 3.33×10⁹ total bacteria (BT) per dose of inactivated B. hyodysenteriae strain B-7843, USA isolate. This USA isolate showed a genomic arrangement consistent with the gene cluster structure S3 (as determined in Example 1). The vaccine was formulated at 50% v/v with the commercial adjuvant Montanide IMS 1313.
Group D corresponded to the positive control group, which received a mock vaccine comprising PBS without antigen and 50% v/v of the same Montanide IMS 1313 adjuvant (mock-vaccinated-challenged group).
Group E corresponded to the negative control group which received a mock vaccine comprising PBS without antigen and 50% v/v of the same Montanide IMS 1313 adjuvant. This group did not receive the experimental infection. Therefore, this group served as strict negative control group (mock-vaccinated-non-challenged group).

All B. hyodysenteriae strains used for preparing the experimental vaccines were inactivated with formaldehyde before administering the vaccine to the animals.

At Day 42, pigs were experimentally infected (challenged) with a fresh culture of B. hyodysenteriae strain B-7493, which is an American isolate with a gene cluster structure S1. The challenge consisted of orally administering 3 doses at 3 consecutive days of 100 mL/dose of a culture of B. hyodysenteriae strain B-7493 at a titer of 1×10⁹ spirochetes/dose per animal by an esophageal cannula. The negative control group (Group E) did not receive the experimental infection.

To assess the efficacy of the vaccines after the experimental infection, it was evaluated the appearance of mucohemorrhagic diarrhea and the intestinal lesion score in all groups as main efficacy parameters. The appearance of mucohemorrhagic diarrhea was considered as the main clinical sign of swine dysentery.

Diarrhea was monitored on a daily basis from day 1 to day 20 post-infection. Subsequently, a weighted diarrhea score was calculated.

Diarrhea score was evaluated according to the following table:

TABLE 2
Diarrhea score

— Normal stool (no diarrhea)
1 pasty stool (consistency of cement, mild diarrhea)
2 liquid stool with fecal content (moderate diarrhea)
3 watery stool (severe diarrhea, does not look like stool)

If presence of mucus is detected it was added 0.5 points to the diarrhea score, and another 0.5 points for the presence of blood. Thus, the diarrhea score ranged from 0 to 4.

Then, the mean for the different groups was calculated.

Another parameter assessed to determine the efficacy of the vaccines was the intestinal lesion score. Animals were euthanized on day 64 of the study and assessed for large intestine intestinal lesions. The score was calculated according to the following table:

TABLE 3
Intestinal lesion score

0 Absence of lesion
1 Mild enteritis, lesions in specific areas, normal or pasty content
2 Moderate enteritis, extensive lesions, diarrheal content
3 Severe enteritis, severely damaged or atrophic intestine, watery/
blood stools, fibrin

The mean of the intestinal lesion score per group was calculated.

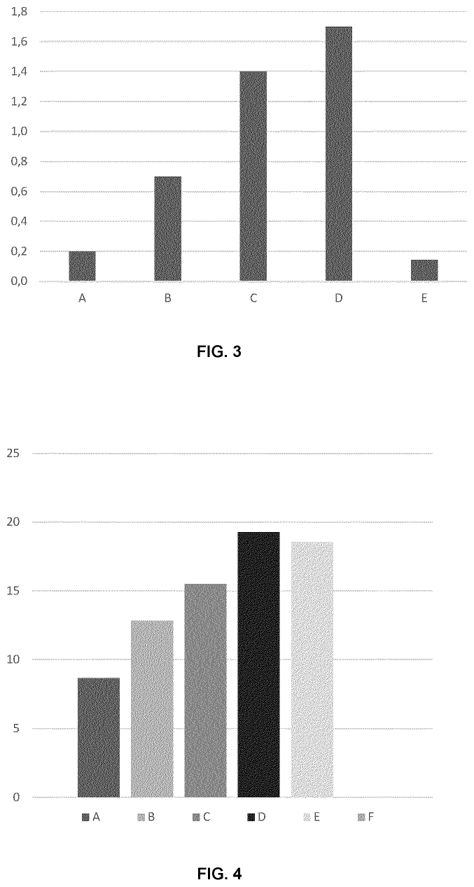

In Groups A and B, a clear reduction of diarrhea and intestinal lesions was observed (FIGS. 2 and 3). Significant efficacy of the vaccines with B. hyodysenteriae strains having the genomic structure S1 was observed. Even if the Bhyo strain is heterologous to the challenge strain (as in Group B). In contrast, the vaccine comprising a B. hyodysenteriae strain having the genomic structure S3 (Group C), did not show protection against the experimental infection as this group performed similar to the positive control group (mock-vaccinated-challenged group).

To sum up, after experimental infection with the inoculum of the B. hyodysenteriae B-7493 strain, it is observed that vaccines with inactivated B. hyodysenteriae strains having the genomic structure S1 significantly reduced the clinical parameters associated to swine dysentery as the diarrhea and intestinal lesion scores. It is further demonstrated that strains having the genomic structure S1 are able to confer cross-protection against heterologous Bhyo strains.

### Example 3: Efficacy Study of Experimental Vaccines Against Experimental Infection of B. hyodysenteriae Strain B-7484

A total of 57 pigs between 5 and 6 weeks of age were chosen for this study. The animals were randomly assigned into 6 groups (Groups A to F) of 10 pigs each one, except for Group F in which only 7 animals were allotted. On Day 0, pigs received a first dose of an experimental vaccine according to the group assignment. Three weeks later (Day 21), pigs received a second dose of the vaccine. Vaccines were administered at the neck by intramuscular route at 2 mL for each administration.
Group A received a vaccine comprising 8×10⁹ total bacteria (BT) per dose of the inactivated B. hyodysenteriae strain B-7500, Spanish isolate. This Spanish isolate showed a genomic arrangement consistent with the gene cluster structure S1 (as determined in Example 1). The vaccine was formulated at 50% v/v with a commercial adjuvant Montanide IMS 1313.
Group B received a vaccine comprising 8×10⁹ total bacteria (BT) per dose of the inactivated B. hyodysenteriae strain B-7484, Spanish isolate. This Spanish isolate showed a genomic arrangement consistent with the gene cluster structure S2 (as determined in Example 1). This vaccine was formulated with the same strain as the one used for the experimental infection, thus it is a homologous vaccine. The vaccine was also formulated at 50% v/v with the commercial adjuvant Montanide IMS 1313.
Group C received a vaccine comprising 8×10⁹ total bacteria (BT) per dose of the inactivated B. hyodysenteriae strain B-7494, Spanish isolate. This Spanish isolate showed a genomic arrangement consistent with the gene cluster structure S2 (as determined in Example 1). The vaccine was formulated at 50% v/v with the commercial adjuvant Montanide IMS 1313.
Group D received a vaccine comprising 8×10⁹ total bacteria (BT) per dose of inactivated B. hyodysenteriae strain B-7843, USA isolate. This USA isolate showed a genomic arrangement consistent with the gene cluster structure S3 (as determined in Example 1). The vaccine was formulated at 50% v/v with the commercial adjuvant Montanide IMS 1313.
Group E corresponded to the positive control group, which received a mock vaccine comprising PBS without antigen and 50% v/v of the same adjuvant Montanide IMS 1313 (mock-vaccinated-challenged group).
Group F corresponded to the negative control group which received a mock vaccine comprising PBS without antigen and 50% v/v of the same Montanide IMS 1313 adjuvant. This group did not receive the experimental infection. Therefore, the group F worked as strict negative control group (mock-vaccinated-non challenged group).

All B. hyodysenteriae strains used for preparing the experimental vaccines were inactivated with formaldehyde before administering the vaccine to the animals.

At Day 41, pigs were experimentally infected (challenged) with a fresh culture of the very virulent B. hyodysenteriae strain B-7484, which is a Spanish isolate with a gene cluster structure S2. The challenge consisted of orally administering 2 doses at 2 consecutive days of 100 mL/dose of the culture of B. hyodysenteriae strain B-7484 at a titer of 1×10⁹ spirochetes/dose per animal by an esophageal cannula. The negative control group (Group F) did not receive the experimental infection.

To assess the efficacy of the vaccines after the experimental infection, it was evaluated the appearance of mucohemorrhagic diarrhea. The appearance of mucohemorrhagic diarrhea was considered as the main clinical sign of swine dysentery. Diarrhea was monitored on a daily basis from day 1 to day 10 post-infection. Then, a weighted diarrhea score was obtained as detailed in Example 2.

The results show a reduction on the diarrhea score in Groups A, B and C (FIG. 4). Thus, there is efficacy of vaccines comprising inactivated Bhyo strains having the genomic structure S1 or S2. Surprisingly, cross-protection is also observed, as efficacy was obtained even if the vaccine strain was heterologous to the challenge strain (Groups A and C).

Vaccines comprising inactivated Bhyo strains having the genomic structure S1 (Group A) resulted in higher efficacy compared to the homologous vaccine strain (Group B). On the contrary, the vaccine comprising a B. hyodysenteriae strain having the genomic structure S3 (Group D), did not confer protection against the experimental infection as this group performed similar to the positive control group (mock-vaccinated-challenged).

### Example 4: Efficacy Study of Experimental Multivalent Vaccines Against Experimental Infection of B. hyodysenteriae Strain B-7484

A total of 66 pigs between 4 and 5 weeks of age were chosen for this study. The animals were randomly assigned into 6 groups of 11 pigs each one (A to E). On Day 0, pigs received a first dose of an experimental vaccine according to the group assignment. Three weeks later (Day 21) pigs received a second dose of the vaccine. Vaccines were administered at the neck by intramuscular route at 2 mL for each administration.
Group A received a vaccine comprising 1×10¹⁰ total bacteria (BT) per dose of the inactivated B. hyodysenteriae strain B-7500, Spanish isolate and 1×10¹⁰ BT per dose of the inactivated B. hyodysenteriae strain B-8295, Argentinian isolate. Both, the Spanish isolate and the Argentinian isolate showed a genomic arrangement consistent with the gene cluster structure S1 (as determined in Example 1). The vaccine was formulated at 2% (w/v) with DEAE-Dextran, and 30% (w/v) with Aluminum hydroxide gel as adjuvant.
Group B received a vaccine comprising 1×10¹⁰ total bacteria (BT) per dose of the inactivated B. hyodysenteriae strain B-7500, which is a Spanish isolate, 1×10¹⁰ BT per dose of the inactivated B. hyodysenteriae strain B-8295, which is an Argentinian isolate and 1×10¹⁰ BT per dose of the inactivated B. hyodysenteriae strain B-8397, which is a German isolate. Both, the Spanish isolate and the Argentinian isolate showed a genomic arrangement consistent with the gene cluster structure S1 (as determined in Example 1); and the German isolate B-8397 holds a genomic arrangement consistent with the gene cluster structure S2 (as determined in Example 1). The vaccine was formulated at 2% (w/v) with DEAE-Dextran, and 30% (w/v) with Aluminum hydroxide gel as adjuvant.
Group C received a vaccine comprising 1×10¹⁰ total bacteria (BT) per dose of the inactivated B. hyodysenteriae strain B-7500, 1×10¹⁰ BT per dose of the inactivated B. hyodysenteriae strain B-8295, 1×10¹⁰ BT per dose of the inactivated B. hyodysenteriae strain B-8397, and 1×10¹⁰ BT per dose of the inactivated B. hyodysenteriae strain B-7484. This last isolate B-7484 corresponds to a Spanish isolate having a genomic arrangement consistent with the gene cluster structure S2 (as determined in Example 1). The vaccine was formulated at 2% (w/v) with DEAE-Dextran, and 30% (w/v) with Aluminum hydroxide gel as adjuvant.
Group D received a vaccine comprising 1×10¹⁰ total bacteria (BT) per dose of the inactivated B. hyodysenteriae strain B-7500, 1×10¹⁰ BT per dose of the inactivated B. hyodysenteriae strain B-8295, 1×10¹⁰ BT per dose of the inactivated B. hyodysenteriae strain B-8397, 6.69×10⁹ BT per dose of the inactivated B. hyodysenteriae strain B-7484, and 1×10¹⁰ BT per dose of the inactivated B. hyodysenteriae strain B-7482. This last isolate B-7482 corresponds to a Spanish isolate having a genomic arrangement consistent with the gene cluster structure S1 (as determined in Example 1). The vaccine was formulated at 2% (w/v) with DEAE-Dextran, and 30% (w/v) with Aluminum hydroxide gel as adjuvant.
Group E corresponded to the control group which received a mock vaccine comprising PBS without antigen and formulated at 2% (w/w) of DEAE-Dextran, and 30% (w/v) of Aluminum hydroxide gel (mock-vaccinated-challenged group).

All B. hyodysenteriae strains used for preparing the experimental vaccines were inactivated with formaldehyde before administering the vaccine to the animals.

At Day 36, pigs were experimentally infected (challenged) with a fresh culture of the very virulent B. hyodysenteriae strain B-7484, which is a Spanish isolate with a gene cluster structure consistent with S2. The challenge consisted of orally administering 3 doses at 3 consecutive days of 100 mL/dose of the culture of B. hyodysenteriae strain B-7484 at a titer about 3×10¹⁰ alive spirochetes/dose per animal by an esophageal cannula.

To assess the efficacy of the vaccines after the experimental infection, different parameters were evaluated: (i) pig survival, it was monitored on daily basis from day 1 to day 20 post-infection, (ii) average daily weight gain from day 1 to day 23 post-infection was also calculated, and (iii) intestinal lesion score, animals were euthanized on day 58 of the study and the intestinal lesion score was obtained. The score was calculated based on the following table. Then, the mean intestinal lesion score for the different groups was calculated.

TABLE 4
Score to assess the intestinal lesion score

0 Absence of lesion
1 Mild enteritis, lesions in specific areas, normal or pasty content
2 Moderate enteritis, extensive lesions, diarrheal content
3 Severe enteritis, severely damaged or atrophic intestine, watery/
blood stools, fibrin
4 Very severe enteritis, totally atrophic intestine, completely
ulcerated mucosa

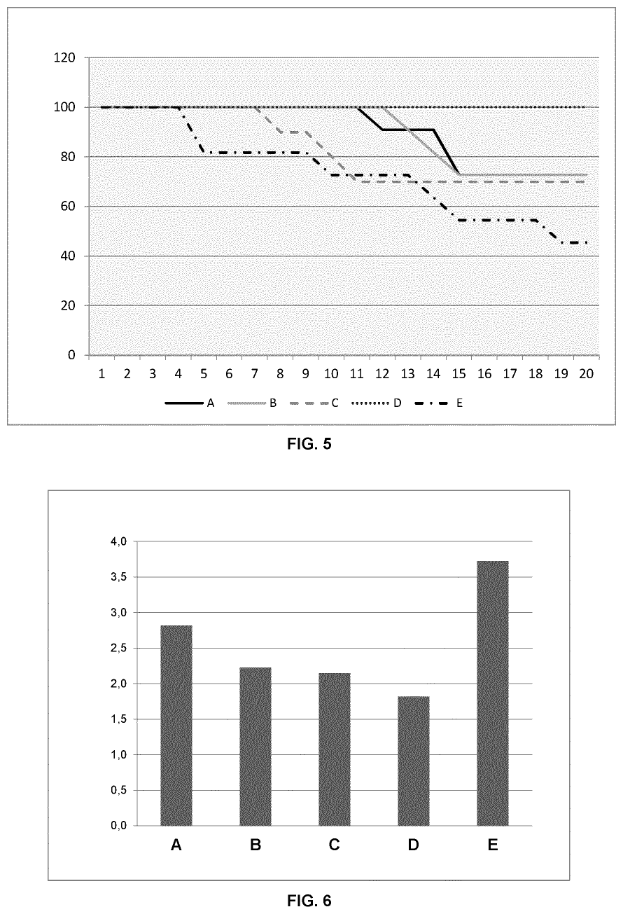

Pig survival and intestinal lesion score results are shown in FIGS. 5 to 6. The results reveal that multivalent Bhyo experimental vaccines administered to all groups (A to D) are effective, they all show efficacy against swine dysentery. Particularly, all vaccinated groups present higher survival rate and reduced intestinal lesion scores when compared to control group E.

Average daily weight gain results are shown in Table 5. For this specific parameter, the results demonstrate that animals vaccinated with multivalent experimental vaccines comprising Bhyo isolates having different genomic arrangement (Groups B to D) increased the average daily weight gain over the study and got a superior final weight gain after 23 days post infection when compared to control group E. In Group A (animals vaccinated with an experimental vaccine comprising two isolates having the same genomic arrangement consistent with the gene cluster S1, it was observed that some animals increased their weight over the study, however the mean did not result in an increase of the average daily weight gain. Nevertheless, Group A performed better in terms of average daily weight gain when compared to the control group (Group E). Overall, and taking into account that this group had also a high percentage of survival rate and a reduced intestinal lesion score when compared to control group E, indicates that the vaccine of Group A also protects pigs affected from swine dysentery from weight loss.

TABLE 5
Mean of the Average daily weight gain and total weight
gain in kilograms until day 23 after the experimental
infection (day 58 of the study), groups A to E.

| Group | Average Daily Weight Gain (Kg) | Weight gain (Kg) |
|---|---|---|
| Group A | −0.03 | −0.80 |
| Group B | 0.14 | 3.2 |
| Group C | 0.11 | 2.5 |
| Group D | 0.06 | 1.6 |
| Group E | −0.09 | −2.1 |

Furthermore, it was surprisingly observed that multivalent vaccines comprising a combination of Bhyo strains having the 36 kb plasmid pBH with the genomic arrangement consistent with the gene cluster structure S1 and S2 (Groups B to D) had higher efficacy and improved protection than vaccines comprising Bhyo strains having just one genomic arrangement, in particular comprising isolates having the genomic arrangement consistent with the gene cluster S1 (Group A). Animals in Groups B to D showed an increased survival rate, higher average daily weight gain and a reduced intestinal lesion score than animals in Group A. This higher efficacy and better protection was shown even when the strain used for the experimental infection is not present in the vaccine composition (Group B).

Overall the results indicate that a vaccine comprising B. hyodysenteriae strains having the genomic arrangement consistent with the gene cluster structure S1 and/or S2 are effective against swine dysentery and that cross-protection against B. hyodysenteriae infections from heterologous strains is attained.

### Example 5: Efficacy Study of Experimental Trivalent Vaccine Against Experimental Infection with Different Heterologous B. hyodysenteriae Strains

A total of 66 pigs between 4 and 5 weeks of age were chosen for this study. The animals were randomly assigned into 6 groups of 11 pigs each one (A to F). On Day 0, pigs received a first dose of an experimental vaccine according to the group assignment. Two weeks later (Day 14) pigs received a second dose of the vaccine. Vaccines were administered at the neck by intramuscular route at 2 mL for each administration.
Groups A, C and E received a vaccine comprising 1.5×10¹⁰ total bacteria (BT) per dose of the inactivated B. hyodysenteriae strain B-7500, which is a Spanish isolate having a genomic arrangement consistent with the gene cluster structure S1 (as determined in Example 1), 1.5×10¹⁰ BT per dose of the inactivated B. hyodysenteriae strain B-8295, which is an Argentinian isolate having a genomic arrangement consistent with the gene cluster structure S1 (as determined in Example 1), and 1.5×10¹⁰ BT per dose of the inactivated B. hyodysenteriae strain B-8397, which is a German isolate having a genomic arrangement consistent with the gene cluster structure S2 (as determined in Example 1). The vaccine was formulated at 2% (w/v) with DEAE-Dextran, and 30% (w/v) with Aluminum hydroxide gel as adjuvant.
Group B, D and F corresponded to control groups which received a mock vaccine comprising PBS formulated at 2% (w/v) with DEAE-Dextran, and 30% (w/v) with Aluminum hydroxide gel as adjuvant. Each group acted as the control group for the vaccinated Groups A, C and E, respectively.

All B. hyodysenteriae strains used for preparing the experimental vaccines were inactivated with formaldehyde before administering the vaccine to the animals.

At Day 36, pigs were experimentally infected (challenged) with a fresh culture of different heterologous B. hyodysenteriae strains. Groups A and B were experimentally infected with a fresh culture of the B. hyodysenteriae strain B-7493, which is a USA isolate having a genomic arrangement consistent with the gene cluster structure S1 (as determined in Example 1). Groups C and D were experimentally infected with a fresh culture of the B. hyodysenteriae strain B-7482, which is a Spanish isolate having a genomic arrangement consistent with the gene cluster structure S1 (as determined in Example 1). Groups E and F were experimentally infected with a fresh culture of the B. hyodysenteriae strain B-7484, which is a Spanish isolate having a genomic arrangement consistent with the gene cluster structure S2 (as determined in Example 1).

The challenge consisted of orally administering 2 doses at 2 consecutive days of 100 mL/dose of the fresh culture of B. hyodysenteriae strain at a titer between 1.4×10¹⁰ and 3.6×10¹⁰ spirochetes/dose per animal by an esophageal cannula.

To assess the efficacy of the vaccines after the experimental infection different parameters were evaluated: (i) pig survival, it was monitored on daily basis from day 1 to day 20 post-infection, and (ii) intestinal lesion score, animals were euthanized on day 57 of the study and the intestinal lesion score was assessed. The score was calculated as described in Example 4.

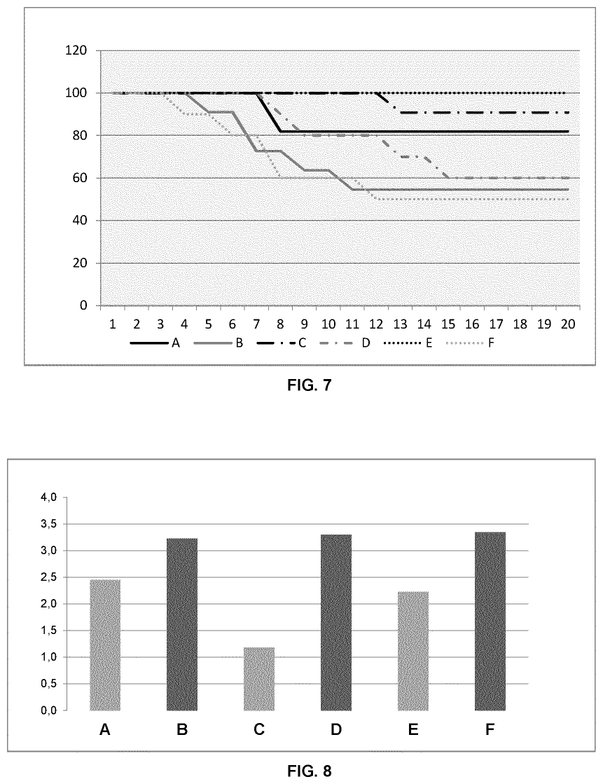

Results are shown in FIGS. 7 and 8.

The results clearly show that all vaccinated groups had higher survival rate, more than 80% survival at day 20 post-infection, and also showed a reduced intestinal lesion score when compared to the control groups. This results surprisingly demonstrate not only protection against swine dysentery but also the cross-protection traits of the experimental trivalent vaccine, as the vaccine is efficacious even with heterologous strains of different geographical origins. Therefore, the results confirm, again, that a vaccine comprising inactivated B. hyodysenteriae strains having the genomic cluster structure S1 and/or S2 are highly effective against swine dysentery and in obtaining cross-protection against B. hyodysenteriae infections, even if the infections are caused by heterologous strains and of different geographic origins.

1-41. (canceled)

42. An immunogenic or vaccine composition comprising an isolated and inactivated bacterium from the species Brachyspira hyodysenteriae, wherein said bacterium comprises the gene on the locus BHWA1_02689 (fucT) of the B. hyodysenteriae plasmid pBHWA1 identified by the GenBank accession number NC_012226.1, wherein said gene has the sequence of SEQ ID NO: 5, or a variant of said gene of any strain of B. hyodysenteriae.

43. The immunogenic or vaccine composition according to claim 42, wherein the variant of the fucT gene has at least 60% sequence identity with the sequence of SEQ ID NO: 5.

44. The immunogenic or vaccine composition according to claim 43, wherein the variant of the fucT gene comprises a sequence selected from the group consisting of SEQ ID NO: 17-31.

45. The immunogenic or vaccine composition according to claim 42, wherein the fucT gene or the variant of said gene is comprised in a gene cluster, and wherein the bacterium is a S1 or a S2 bacterium, wherein
the gene cluster of the S1 bacterium comprises the following genes: the gene on the locus BHWA1_02695 (bcsA) of the B. hyodysenteriae plasmid pBHWA1 identified by the GenBank accession number NC_012226.1, wherein said gene has the sequence of SEQ ID NO: 7, or a variant of said gene of any strain of B. hyodysenteriae, the rfbC gene, the rfbD gene, the rfbA gene, the rfbB gene, the gene on the locus BHWA1_02690 (rgpF) of the B. hyodysenteriae plasmid pBHWA1 identified by the GenBank accession number NC_012226.1, wherein said gene has the sequence of SEQ ID NO: 6, or a variant of said gene of any strain of B. hyodysenteriae and the fucT gene or a variant of said gene of any strain and
the gene cluster of the S2 bacterium comprises the following genes: the gene on the locus BHWA1_02695 (bcsA) of the B. hyodysenteriae plasmid pBHWA1 identified by the GenBank accession number NC_012226.1, wherein said gene has the sequence of SEQ ID NO: 7, or a variant of said gene of any strain of B. hyodysenteriae, the rfbC gene, the rfbD gene, the rfbA gene, a gene having the sequence of SEQ ID NO: 8 (adoM) or a variant of said gene of any strain of B. hyodysenteriae, the rfbB gene, the gene on the locus BHWA1_02690 (rgpF) of the B. hyodysenteriae plasmid pBHWA1 identified by the GenBank accession number NC_012226.1, wherein said gene has the sequence of SEQ ID NO: 6, or a variant of said gene of any strain of B. hyodysenteriae and the fucT gene or a variant of said gene of any strain of B. hyodysenteriae.

46. The immunogenic or vaccine composition according to claim 45, wherein
the variant of the rgpF gene has at least 60% sequence identity with the sequence of SEQ ID NO: 6, and/or,
the variant of the bcsA gene has at least 60% sequence identity with the sequence of SEQ ID NO: 7, and/or
the variant of the adoM gene has at least 60% sequence identity with the sequence of SEQ ID NO: 8.

47. The immunogenic or vaccine composition according to claim 46, wherein
the variant of the rgpF gene comprises a sequence selected from the group consisting of SEQ ID NO: 69-76 and/or
the variant of the bcsA gene comprises a sequence selected from the group consisting of SEQ ID NO: 77-81 and/or
the variant of the adoM gene comprises the sequence of SEQ ID NO: 82.

48. The immunogenic or vaccine composition according to claim 45, wherein
the gene cluster of the S1 bacterium comprises, from the 5′ to the 3′ end, the following genes: bcsA or a variant of said gene of any strain of B. hyodysenteriae, rfbC, rfbD, rfbA, rfbB, rgpF or a variant of said gene of any strain of B. hyodysenteriae and fucT or a variant of said gene of any strain of B. hyodysenteriae; and
the gene cluster of the S2 bacterium comprises, from the 5′ to the 3′ end, the following genes: bcsA or a variant of said gene of any strain of B. hyodysenteriae, rfbC, rfbD, rfbA, adoM or a variant of said gene of any strain of B. hyodysenteriae, rfbB, rgpF or a variant of said gene of any strain of B. hyodysenteriae and fucT or a variant of said gene of any strain of B. hyodysenteriae.

49. The immunogenic or vaccine composition according to claim 45, wherein the composition comprises at least two strains of a S1 bacterium.

50. The immunogenic or vaccine composition according to claim 45, wherein the composition comprises at least two strains of a S2 bacterium.

51. The immunogenic or vaccine composition according to claim 45, wherein the composition comprises:
at least two strains of a S1 bacterium and at least one strain of a S2 bacterium or
at least one strain of a S1 bacterium and at least two strains of a S2 bacterium or
at least two strains of a S1 bacterium and at least two strains of a S2 bacterium.

52. The immunogenic or vaccine composition according to claim 51, wherein the composition comprises:
the strain B-7500, deposited by in the Leibniz-Institut DSMZ-Deutsche Sammlung von Mikroorganismen und Zellkulturen GmbH under accession number DSM 33602,
the strain B-8295, deposited in the Leibniz-Institut DSMZ-Deutsche Sammlung von Mikroorganismen und Zellkulturen GmbH under accession number DSM 33603 and
the strain B-8397, deposited in the Leibniz-Institut DSMZ-Deutsche Sammlung von Mikroorganismen und Zellkulturen GmbH under accession number DSM 33604.

53. The immunogenic or vaccine composition according to claim 42, further comprising an adjuvant.

54. The immunogenic or vaccine composition according to claim 53, wherein the adjuvant is an aluminum containing adjuvant, diethylaminoethyl (DEAE) or a combination thereof.

55. The immunogenic or vaccine composition according to claim 42, wherein the bacterium has been inactivated by formaldehyde.

56. A method for producing an immunogenic or vaccine composition comprising an isolated bacterium from the species Brachyspira hyodysenteriae, wherein the method comprises the steps of:
(a) determining if the bacterium from the species Brachyspira hyodysenteriae comprises the gene on the locus BHWA1_02689 (fucT) of the B. hyodysenteriae plasmid pBHWA1 identified by the GenBank accession number NC_012226.1, wherein said gene has the sequence of SEQ ID NO: 5, or a variant of said gene of any strain of B. hyodysenteriae, or the product of said gene or variant,
(b) selecting the bacterium from the species Brachyspira hyodysenteriae if said bacterium comprises the fucT gene or a variant of said gene of any strain of B. hyodysenteriae or the product of said gene or variant,
(c) inactivating the bacterium selected in step (b) and
(d) formulating the bacterium inactivated in step (c) in an immunogenic or vaccine composition.

57. An immunogenic or vaccine composition obtainable by the method according to claim 56.

58. A method for selecting a bacterium from the species Brachyspira hyodysenteriae useful for manufacturing a vaccine against swine dysentery comprising determining the presence or absence of the gene on the locus BHWA1_02689 (fucT) of the B. hyodysenteriae plasmid pBHWA1 identified by the GenBank accession number NC_012226.1, wherein said gene has the sequence of SEQ ID NO: 5, or a variant of said gene of any strain of B. hyodysenteriae and/or of the product of said gene or variant, wherein if the bacterium comprises said gene or variant or the product of said gene or variant, the bacterium is selected for manufacturing a vaccine against swine dysentery.

59. A method for inducing an immune response against a bacterium from the species Brachyspira hyodysenteriae comprising administering to a subject an immunologically or therapeutically effective amount of the immunogenic composition according to claim 42.

60. A method for preventing an infection caused by a bacterium from the species Brachyspira hyodysenteriae in a subject comprising administering to a subject an immunologically or therapeutically effective amount of the vaccine composition according to claim 42.

61. The method according to claim 60, wherein the vaccine induces protection against a strain of B. hyodysenteriae different from the strain that is comprised in the vaccine.
